(19) United States
(12) Patent Application Publication
(10) Pub. No.: US 2013/0045939 A1
(43) Pub. Date: Feb. 21, 2013

(54) FATTY ACID MACROLIDE DERIVATIVES AND THEIR USES

(71) Applicants: Chi B. Vu, Arlington, MA (US); Jean E. Bemis, Arlington, MA (US); Michael R. Jirousek, Cambridge, MA (US); Jill C. Milne, Brookline, MA (US)

(72) Inventors: Chi B. Vu, Arlington, MA (US); Jean E. Bemis, Arlington, MA (US); Michael R. Jirousek, Cambridge, MA (US); Jill C. Milne, Brookline, MA (US)

(73) Assignee: CATABASIS PHARMACEUTICALS, INC., Cambridge, MA (US)

(21) Appl. No.: 13/636,020

(22) PCT Filed: Mar. 18, 2011
(86) PCT No.: PCT/US2011/029042

§ 371 Date: Oct. 19, 2012

Related U.S. Application Data
(60) Provisional application No. 61/315,626, filed on Mar. 19, 2010.

(51) Int. Cl. C07D 267/00; C07D 413/12; A61K 31/7052; A61P 11/00; A61P 29/00; A61P 19/02; A61P 1/00; A61P 11/06; C07H 17/00; A61K 31/395

(52) U.S. Cl. 514/29; 536/7.1; 540/467; 514/450

(57) ABSTRACT

The invention relates to fatty and macrolide derivatives; compositions comprising an effective amount of fatty acid macrolide derivative; and methods for treating or preventing an autoimmune disorders and diseases with inflammation as the underlying etiology comprising the administration of an effective amount of a fatty acid macrolide derivative.

### PRIORITY

The present application claims the benefit of U.S. Provisional Application No. 61/315,626 filed Mar. 19, 2010, the entire disclosure of which is relied on for all purposes and is incorporated into this application by reference.

### FIELD OF THE INVENTION

The invention relates to fatty acid macrolide derivatives, compositions comprising an effective amount of a fatty acid macrolide derivative, and methods for treating or preventing autoimmune disorders and diseases with inflammation as the underlying etiology comprising the administration of an effective amount of a fatty acid macrolide derivative. All patents, patent applications, and publications cited herein are hereby incorporated by reference in their entireties.

### BACKGROUND OF THE INVENTION

Oily cold water fish, such as salmon, trout, herring, and tuna are the source of dietary marine omega-3 fatty acids, with eicosapentaenoic acid (EPA) and docosahexaenoic acid (DHA) being the key marine derived omega-3 fatty acids. Omega-3 fatty acids have previously been shown to improve insulin sensitivity and glucose tolerance in normoglycemic men and in obese individuals. Omega-3 fatty acids have also been shown to improve insulin resistance in obese and non-obese patients with an inflammatory phenotype. Lipid, glucose, and insulin metabolism have been shown to improve in overweight hypertensive subjects through treatment with omega-3 fatty acids. Omega-3 fatty acids such as EPA and DHA have also been shown to decrease triglycerides and to reduce the risk for sudden death caused by cardiac arrhythmias in addition to improving mortality in patients at risk of a cardiovascular event. Omega-3 fatty acids have also been taken as the dietary supplement portion of therapy used to treat dyslipidermia. A higher intake of omega-3 fatty acids lower levels circulating TNF-α and IL-6, two of the cytokines that are markedly increased during inflammation processes (Chapkin et al, Prostaglandins, Leukot Essent Fatty Acid 2009, 81, p. 187-191). In addition, a higher intake of omega-3 fatty acids has been omega-3 fatty acids could be explained, in large part, by inhibition of the NF-κB pathway, which regulates the expression of various pro-inflammatory cytokines, chemokines, cell adhesion molecules and matrix metalloproteinases (Duda, et al. Cardiovasc. Res. 2009, 84, 33-41).

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as free radical scavenger, chelator to heavy metals and signal transduction mediator in various inflammatory and metabolic pathways, including the NF-κB pathway (Shay, K. P. et al. Biochim. Biophys. Acta 2009, 1790, 1149-1160). Lipoic acid has been found to be useful in a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S. et al Neth. J. Med. 2010, 110, p. 158-162). Lipoic acid has also been found to be potentially useful in treating cardiovascular diseases (Ghibu, S. et al, J. Cardiovasc. Pharmacol. 2009, 54, p. 391-8), Alzheimer's disease (Maczurek, A. et al, Adv. Drug Deliv. Rev. 2008, 60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42).

Over the years, macrolides such as azithromycin, erythromycin, clarithromycin, roxithromycin, and telithromycin have been used extensively in the clinic as effective antibacterial agents against a wide range of gram-positive and negative pathogens. More recently, a number of reports show that macrolides exhibit anti-inflammatory properties (Amsen, G. W. J. Antimacrob. Chemother. 2005, 55, 10-21). Some of the anti-inflammatory effects could be attributed to the modulating effect of macrolides upon certain cytokines such as IL-8 and IL-5 (Takizawa, et al, Am. J. Respir. Crit. Care Med. 1997, 156, 266-271; European Pat. App. Nos. 95928005.8 and 95928004.1). In studies involving the use of Lipopolysaccharide (LPS) stimulated J774 macrophages, some macrolides have been shown to reduce the levels of certain proinflammatory mediators and cytokines such as TNF-α, IL-1β and IL-6(Ianaro, et al, J. Pharmacol. Exp. Ther. 2000, 292, 156-163). Clarithromycin, for instance, have been shown to inhibit NF-κB activities in human peripheral blood mononuclear cells and pulmonary epithelial cells (Ichiyama, et al. Antimicrob. Agents Chemother. 2001, 45, 44-47). Azithromycin and erythromycin have been shown to be efficacious in a rat ulcerative colitis model induced by intracolonic administration of 3% acetic acid (Mahgoub, et al. Toxicol. Appl. Pharm. 2005, 205, 43-52). In patients having cystic fibrosis, azithromycin has been shown to improve lung function, body weight and reduced hospital stays (Saiman, et al. J. Am. Med. Ass. 2005, 290, 1749-1756). In cystic fibrosis airway epithelial cells, azithromycin has been shown to reduce TNF-α, and inhibition of NF-κB has been proposed as a possible mechanism of action (Cigana, et al. Antimicrob. Agents Chemother. 2007, 51, 975-981). Lastly, one unique property of macrolides is their ability to accumulate preferentially within phagocyte cells such as mononuclear peripheral blood cells and peritoneal and alveolar macrophages (Olsen et al. Antimicrob. Agents Chemother. 1996, 40, p. 2582-2585). Because of this preferential accumulation in macrophages, macrolides could potentially serve as selective carriers to inflammation sites.

The ability to provide the effects of fatty acid and macrolides in a synergistic way would provide benefits in treating a variety of inflammatory and autoimmune diseases.

### SUMMARY OF THE INVENTION

The invention is based in part on the discovery of fatty acid macrolide derivatives and their demonstrated effects in achieving improved treatment that cannot be achieved by administering macrolides or fatty acids alone or in combination. These novel compounds are useful in the treatment or prevention of autoimmune diseases and diseases with inflammation as the underlying etiology such as rheumatoid arthritis, inflammatory bowel diseases (including ulcerative colitis and Crohn's disease), inflammatory lung diseases such as asthma, adult respiratory distress syndrome, bronchitis, chronic obstructive pulmonary disease (COPD), cystic fibrosis, rheumatoid spondylitis, osteoarthritis, gouty arthritis, uveitis, conjunctivitis, distal proctitis, psoriasis, eczema, dermatitis, coronary infarct damage, chronic inflammation, endotoxin shock, and smooth muscle proliferation disorders.

Accordingly in one aspect, a molecular conjugate is described which comprises a macrolide and a fatty acid wherein the fatty acid is selected from the group consisting of lipoic acid and omega-3 fatty acids and fatty acids that are metabolized in vivo to omega-3 fatty acids, and the conjugate is capable of hydrolysis to produce free macrolide and free fatty acid.

In another aspect, compounds of the Formula I are described:

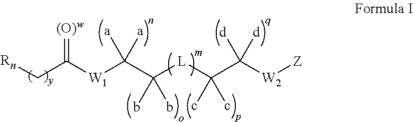

and pharmaceutically acceptable salts, hydrates, solvates, prodrugs, enantiomers, and stereoisomers thereof:

wherein

Rₐ is macrolide;

W₁ and W₂ are each independently null, O, S, NH, NR, or W₁ and W₂ can be taken together can form an imidazolidine or piperazine group;

each a, b, c, and d is independently —H, —D, —CH₃, —OCH₃, —OCH₂CH₃, —C(O)OR, —O-Z, or benzyl, or two of a, b, c, and d can be taken together, along with the single carbon to which they are bound, to form a cycloalkyl or heterocycle;

w is 0 or 1;

y is 0, 1, 2, or 3;

each n, o, p, and q is independently 0, 1 or 2;

L is independently null, —O—, —S—, —S(O)—, —S(O)₂, —S-S—, —(C₁-Ch₆alkyl)-, —(C₃-C₆cycloalkyl)-, a heterocycle, a heteroaryl,

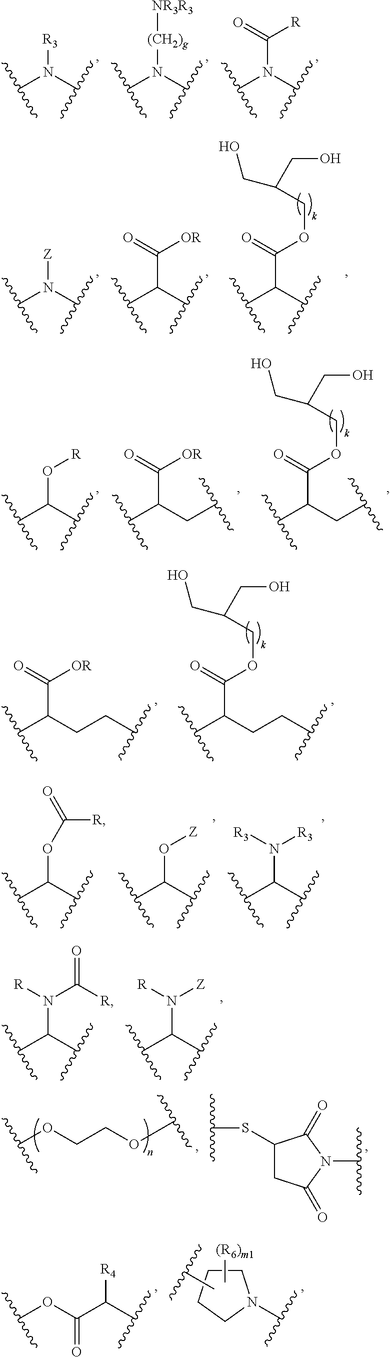

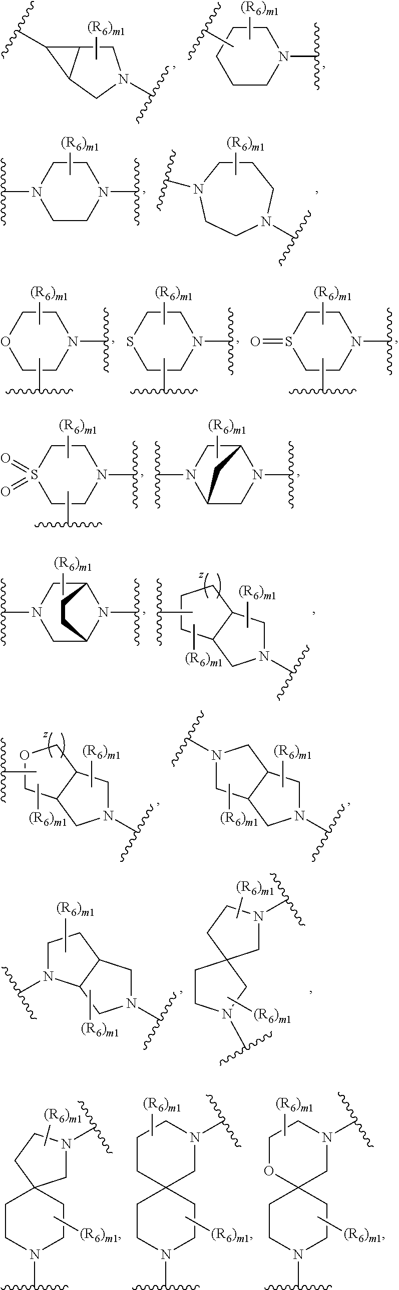

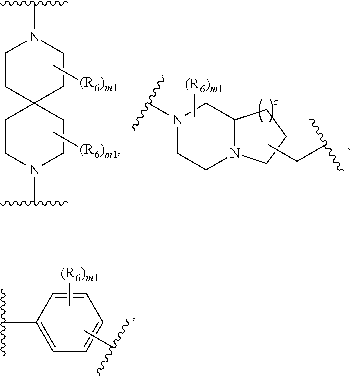

wherein the representation of L is not limited directionally left to right as is depicted, rather either the left side or the right side of L can be bound to the W₁ side of the compound of Formula I;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄, alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3, if m is more than 1, then L can be the same or different;

m1 is 0, 1, 2 or 3;

k is 0, 1, 2, or 3;

z is 1, 2, or 3;

each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ is independently e, H or straight or branched C₃-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H, or

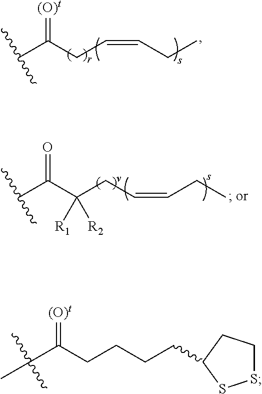

with the proviso that there is at least one

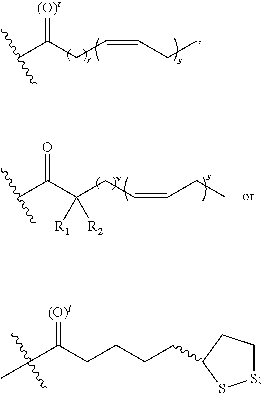

so the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, w is 1, W₁ W₂ are each null, and Z is

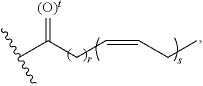

then t must be 0; and
when m, n, o, p, and q are each 0, w is 1, and W₁ and W₂ are each null, then Z must not be

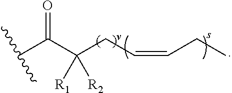

In another aspect, compounds of the Formula Ia are described:

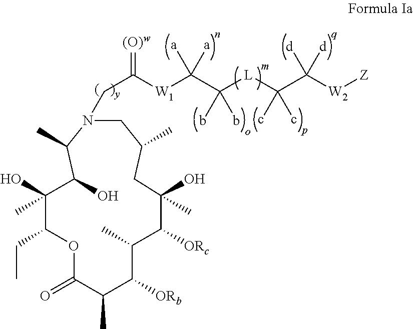

and pharmaceutically acceptable salts, hydrates, solvates, prodrugs, enantiomers, and stereoisomers thereof:

wherein

R_{b} is H, or

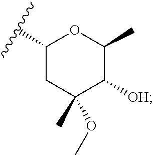 [SMILES: *[C@H]1C[C@@](C)(OC)[C@@H](O)[C@H](C)O1]

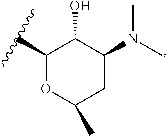 [SMILES: *[C@@H]1O[C@H](C)C[C@H](N(C)C)[C@H]1O]

with the proviso that when R_{c} is H, then R_{b} is H:

W₁ and W₂ are each independently null, O, S, NH, NR, or W₁ and W₂ can be taken together can form an imidazolidine or piperazine group;

each a, b, c, and d is independently —H, -D, —CH₃, —OCH₃, —OCH₂CH₃, —C(O)OR, —O-Z, or benzyl, or two of a, b, c, and d can be taken together, along with the single carbon to which they are bound, to form a cycloalkyl or heterocycle;

w is 0 or 1;

y is 0, 1, 2, or 3;

each n, o, p, and q is independently 0, 1 or 2;

L is independently null, —O—, —S—,—S(O)—, —S(O)₂, —S—S—, —(C₁-C₆alkyl)-, —(C₃-C₆cycloalkyl)-, a heterocycle, a heteroaryl,

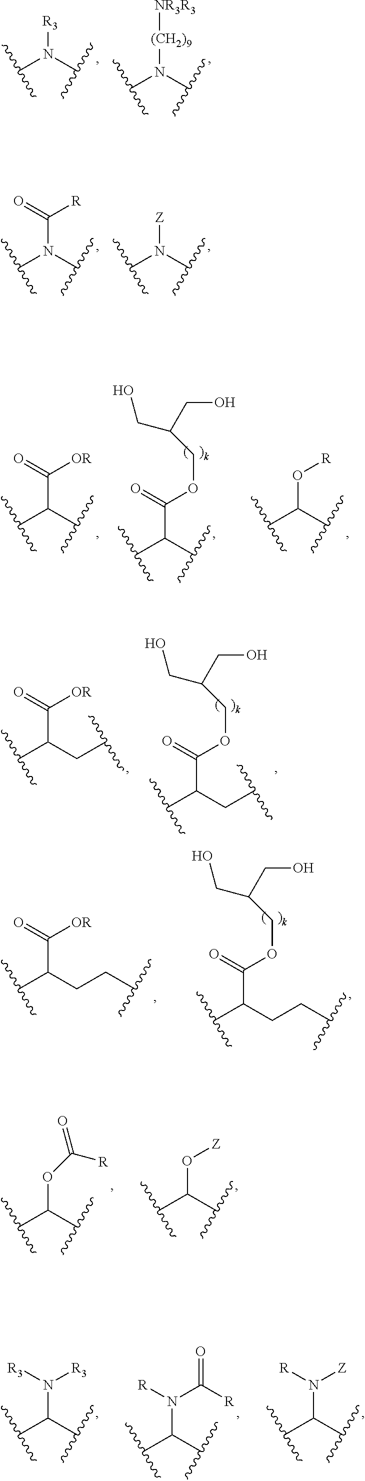

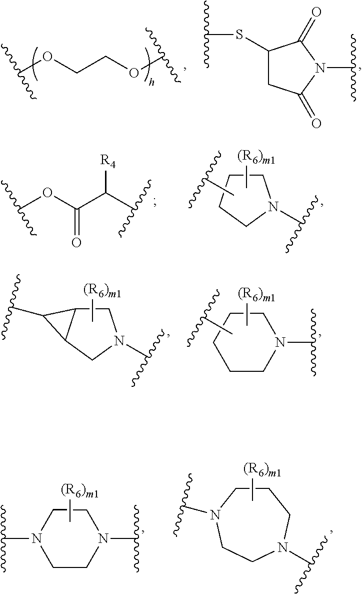

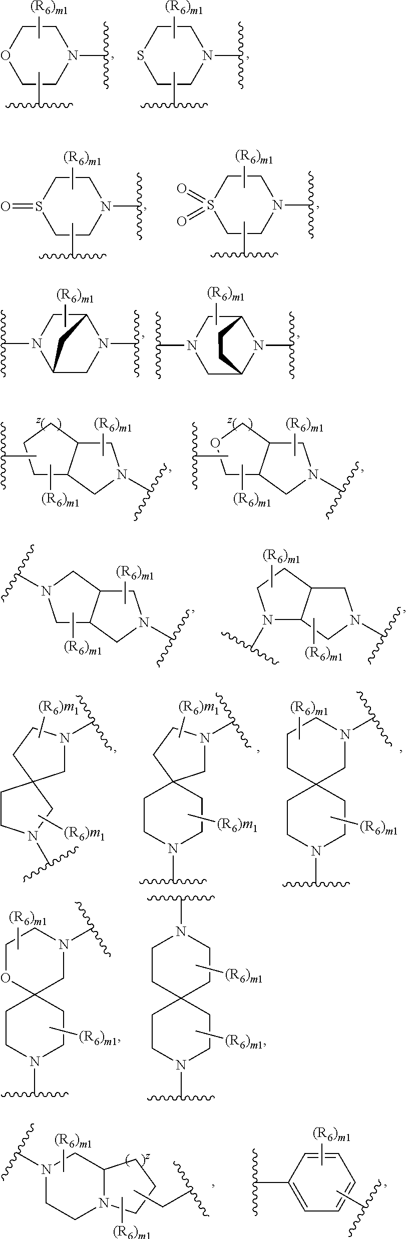

wherein the representation of L is not limited directionally left to right as is depicted, rather either the left side or the right side of L can be bound to the W₁ side of the compound of Formula I;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂,—NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl that can be optionally substituted with either O or N and in NR₃R₃ both R₃ when taken together with the nitrogen to which they are attached can form a heterocyclic ring such as a pyrrolidine, piperidine, morpholine, piperazine or pyrrole;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H, or

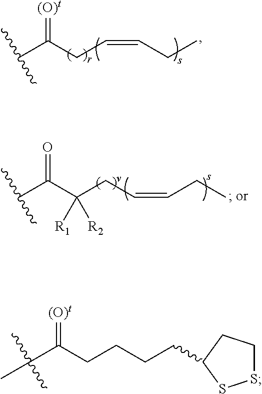

with the proviso that there is at least one

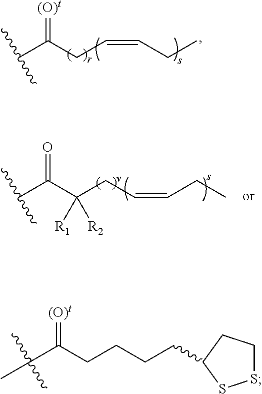

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, w is 1, W₁ and W₂ are each null, and Z is

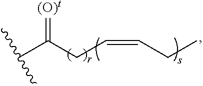

then t must be 0; and
when m, n, o, p, and q are each 0, w is 1, and W₁ and W₂ are each null, then Z must not be

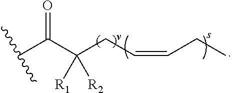

In another aspect, compounds of the Formula Ib are described:

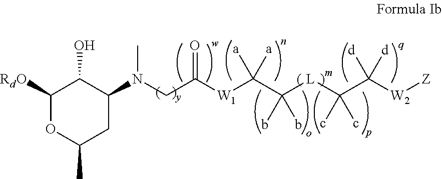

and pharmaceutically acceptable salts, hydrates, solvates, prodrugs, enantiomers, and stereoisomers thereof;

wherein

R_{d is}

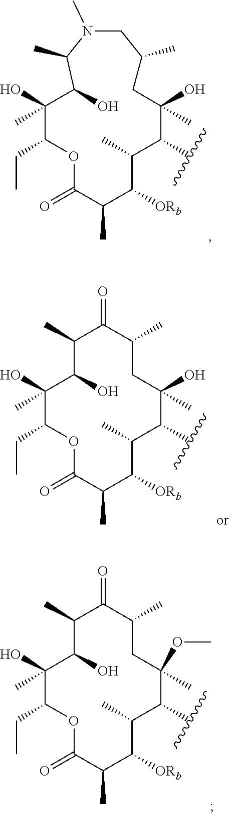

R_{b is H, or}

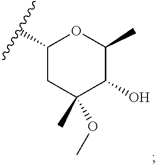 [SMILES: *[C@H]1C[C@@](C)(OC)[C@@H](O)[C@H](C)O1]

W₁ and W₂ are each independently null, O, S, NH, NR, or W₁ and W₂ can be taken together can form an imidazolidine or piperazine groups;

each a, b, c, and d is independently —H, -D, —CH₃, —OCH₃, —OCH₂CH₃, —C(O)OR, —O-Z, or benzyl, or two of a, b, c, and d can be taken together, along with the single carbon to which they are bound, to form a cycloalkyl or heterocycle;

w is 0 or 1;

y is 0, 1, 2, or 3;

each n, o, p, and q is independently 0, 1 or 2;

L is independently null, —O—, —S—, —S(O)—, —S(O)₂-, —S-S—, —(C₁-C₆alkyl)-, —(C₃-C₆-cycloalkyl)-, a heterocycle, a heteroaryl,

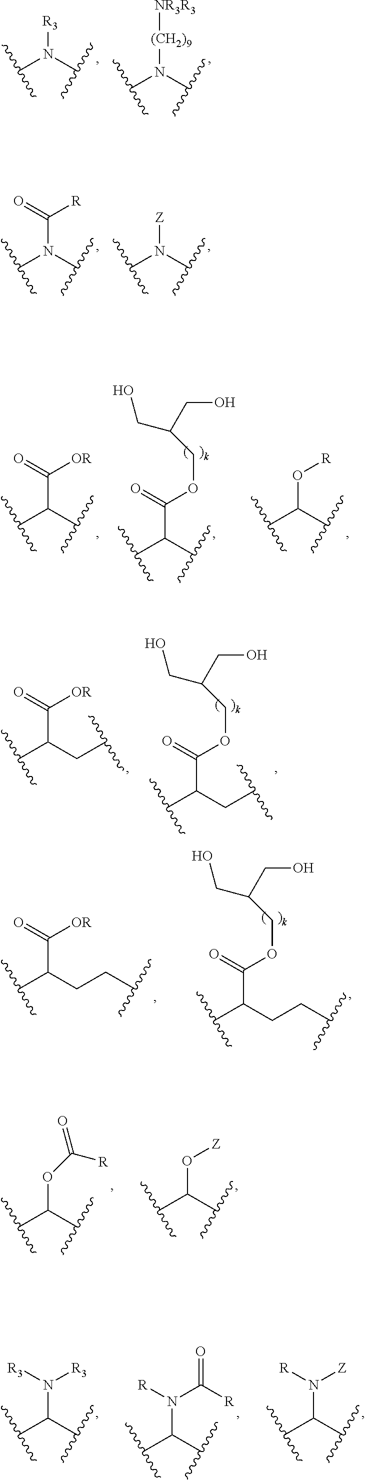

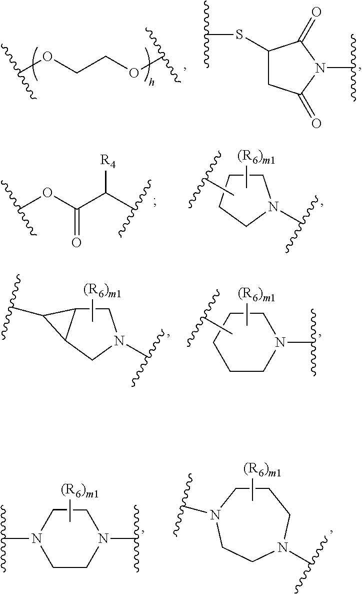

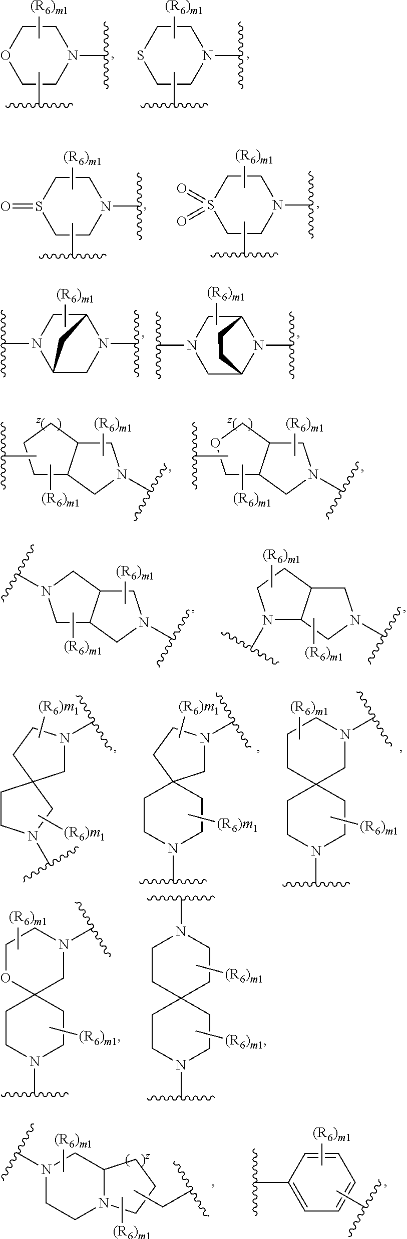

wherein the representation of L is not limited directionally left to right as is depicted, rather either the left side or the right side of L can be bound to the W₁ side of the compound of Formula I;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; is m is more than 1, then L can be the same or different;

m1 0, 1, 2 or 3;

z is 1, 2, or 3;

each R₃ is independently H or C₁C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H, or

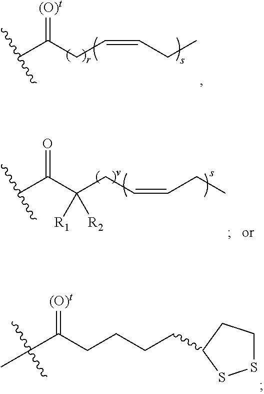

with the proviso that there is at least one

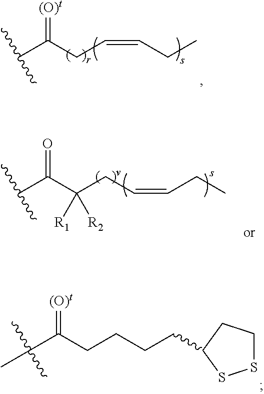

in the compound;

each r is independently 2, 3, or 7;

each s is independently 3, 5, or 6;

each t is independently 0 or 1;

each v is independently 1, 2, or 6;

R₁ and R₂ are each independently hydrogen deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

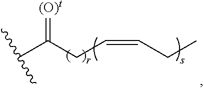

then t must be 0; and
when m, n, o, p, and q are each 0, w is 1, and W₁ and W₂ are each null, then Z must not be

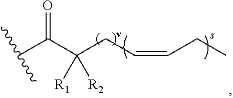

In Formula I, Formula Ia and Formula Ib, any one or more of H may be substituted with a deuterium. It is also understood in Formula I, Formula Ib and Formula Ib, that a methyl substituent can be substituted with a C₁-C₆ alkyl.

Also described are pharmaceutical formulations comprising at least one fatty acid macrolide derivative.

Also described herein are methods of treating a disease susceptible to treatment with a fatty acid macrolide derivative in a patient in need thereof by administering to the patient an effective amount of a fatty acid macrolide derivative.

Also described herein are methods of treating autoimmune diseases or diseases with inflammation as the underlying etiology administering to a patient in need thereof an effective amount of a fatty acid macrolide derivative.

The invention also includes pharmaceutical compositions that comprise an effective amount of a fatty acid macrolide derivative and a pharmaceutically acceptable carrier. The compositions are useful for treating or preventing an autoimmune disease or diseases with inflammation as the underlying etiology. The invention includes a fatty acid macrolide derivative when provided as a pharmaceutically acceptable prodrug, a hydrate, a salt, such as a pharmaceutically acceptable salt, enantiomer, stereoisomer, or mixtures.

The details of the invention are set forth in the accompanying description below. Although method and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### DETAILED DESCRIPTION OF THE INVENTION

Metabolic disorders are a wide variety of medical disorders that interfere with a subject's metabolism. Metabolism is the process a subject's body uses to transform food into energy. Metabolism in a subject with a metabolic disorder is disrupted in some way. Autoimmune diseases arise from an overactive immune response of the body against tissues normally present in the body. The fatty acid macrolide derivatives possess the ability to treat or prevent autoimmune diseases or diseases with inflammation as the underlying etiology.

The fatty acid macrolide derivatives have been designed to bring together a macrolide and omega-3 fatty acids into a single molecular conjugate. The activity of the fatty acid macrolide derivatives is substantially greater than the sum of the individual components of the molecular conjugate, suggesting that the activity induced by the fatty acid macrolide derivatives is synergistic.

Definitions

The following definitions are used in connection with the fatty acid macrolide derivatives.

The term “fatty acid macrolide derivatives” includes any and all possible isomers, stereoisomers, enantiomers, diastereomers, tautomers, pharmaceutically acceptable salts, hydrates, solvates, and prodrugs of the fatty acid macrolide derivatives described herein.

The articles “a” and “an” are used in this disclosure to refer to one or more than one (i.e., to at least on) of the grammatical object of the article. By way of example “an element” means one element or more than one element.

The term “and/or” is used in this disclosure to mean either “and ” or “or” unless indicated otherwise.

Unless otherwise specifically defined, the term “aryl” refers to cyclic, aromatic hydrocarbon groups that have 1 or 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 or 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a cyclic hydrocarbon containing 3-6 atoms wherein at least one of the atoms is an O, N, or S. Examples of a heterocycle group include, but are not limited to, aziridine, oxirane, thiirane, azetidine, oxetane, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane, and dioxane.

The term “heteroaryl” as used herein refers to a monocyclic or bicyclic ring structure having 5 too 12 ring atoms wherein one or more of the ring atoms is a heteroatom, e.g. N, O or S and wherein one or more rings of the bicyclic ring structure is aromatic. Some examples of heteroaryl are pyridyl, furyl, pyrrolyl, thienyl, thiazolyl, oxazolyl, imidazolyl, indolyl, tetrazolyl, benzofuryl, xanthenes and dihydroindole. It is understood that any of the substitutable hydrogens on a heteroaryl can be substituted with halogen, C₁-C₃ alkyl, hydroxyl, alkoxy and cyano groups.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15- octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis- 5,8,11,14,17-eicosapentaenoic acid), docosapentaenoic acid (DPA, clupanodonic acid or all- cis-7,10,13,16,19-docosapentaenoic acid), docosaheaenoic acid (DHA or all cis- 4,7,10,13,16,19-docosahexaenoic acid), tetracosapentaenoic acid (all-cis-9,12,15,18,21- docosahexaenoic acid), and tetracosahexaenoic acid (nisinic acid or all-cis-6,9,12,15,18,21- tetracosenoic acid). In addition, the term “fatty acid” can also refer to medium chain fatty acids such as lipoic acid.

The term “macrolide” as used herein refers to a compound containing a macrocyclic lactone ring having more than 10 atoms in the ring and its derivatives. 14-membered macrolides include erythromycin and its derivatives (such as clarithromycin, roxithromycin and telithromycin). 15-membered macrolides include azithromycin and its derivatives (such as 9-a-N-desmethyl azithromycin, 3′-N-desmethyl azithromycin), as well as 8a- and 9a-lactams and their derivatives.

“Macrolide” as used herein also includes both macrolides which contain a desosamine moiety and/or a cladinose moiety, as well as macrolides lacking both.

A “subject” is a mammal, e.g., a human, mouse, rat, guinea pig, dog, cat, horse, cow, pig, or non-human primate, such as a monkey, chimpanzee, baboon or rhesus, and the terms “subject” and “patient” are used interchangeably herein.

The invention also includes pharamaceutical compositions comprising an effective amount of a fatty acid macrolide derivative and a pharmaceutically acceptable carrier. The invention includes a fatty acid macrolide derivative when provided as a pharmaceutically acceptable prodrug, hydrate, salt, such as a pharmaceutically acceptable salt, enantiomers, stereoisomers, or mixture thereof.

Representative “pharmaceutically acceptable salts” include, e.g., water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, flunarate, fluceptate, gluconate, glutamate, glycollylarsanilate, hexafluorophoshate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, (1,1-methene-bis-2-hydroxy-3-naphthoate, einbonate), pantothenate, phosphate/diphosphate, picrate, polyglalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

An “effective amount” when used in connection with a fatty acid macrolide derivative is an amount effective for treating or preventing an autoimmune diseases or diseases with inflammation as the underlying etiology.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject'disorder. Treating can be curing, improving, or at least partially ameliorating the disorder.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug, ” as used in this disclosure, means a compound which is convertible in vivo by metabolic means (e.g., by hydrolysis) to a fatty acid macrolide derivative.

The following abbreviations are used herein and have the indicated definitions: Cbz is carboxybenzyl, CPS is counts per second, DIEA is N,N-diisopropylethylamine, DMEM is Dulbecco's Modified Eagle Medium, DMSO is dimethyl sulfoxide, DOSS is sodium dioctyl sulfoxuccinate, EDC and EDCI are 1-ethyl-3-(3- dimethylaminopropyl)carbodiimide hydrochloride, ELISA is enzyme-linked immunosorbent assay, EtOAc is ethyl acetate, h is hour, HATU is 2-(7-aza-1H-benzotriazole-1-yl)-1,1,3,3- tetramethyluronium hexafluorophosphate, HPMC is hydroxypropyl methylcellulose, LPS is lipopolysaccharide, NaOAc is sodium acetate, TGPS is tocopherol propylene glycol succinate, and TNF is tumor necrosis factor.

Compounds

The invention is based in part on the discovery of fatty acid macrolide derivatives and their demonstrated effects in achieving improved treatment that cannot be achieved by administering macrolides or fatty acids alone on in combination. These novel compounds are useful in the treatment or prevention of autoimmune diseases such as rheumatoid arthritis, inflammatory bowel diseases (including ulcerative colitis and Crohn's disease), inflammatory lung diseases such as asthma, adult respiratory distress syndrome, bronchitis, chronic obstructive airway disease, cystic fibrosis, rheumatoid spondylitis, osteoarthritis, gouty arthritis, uveitis, conjunctivitis, distal proctitis, psoriasis, eczema, dermatitis, coronary infarct damage, chronic inflammation, endotoxin shock, and smooth muscle proliferation disorders.

Accordingly in one aspect, a molecular conjugate is described which comprises a macrolide and a fatty acid wherein the fatty acid is selected from the group consisting of lipoic acid, omega-3 fatty acids and fatty acids that are metabolized in vivo to omega-3 fatty acids, and the conjugate is capable of hydrolysis to produce free macrolide and free fatty acid.

In another aspect, the present invention provides fatty acid macrolide derivatives according to Formula I:

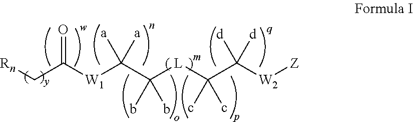

and pharmaceutically acceptable salts, hydrates, solvates, prodrugs, enantiomers, and stereoisomers thereof:

wherein

W₁, W₂, a, b, c, d, e, k, m, m1, n, o, p, q, L, Z, r, s, t, v, w, y, z, Rₐ, R₁, R₂, R₃, R₄, R and R₆ are as defined above for Formula I:

with the proviso that there is as least on

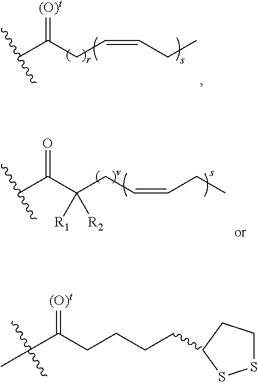

in the compound.

In some embodiment, Rₐ is

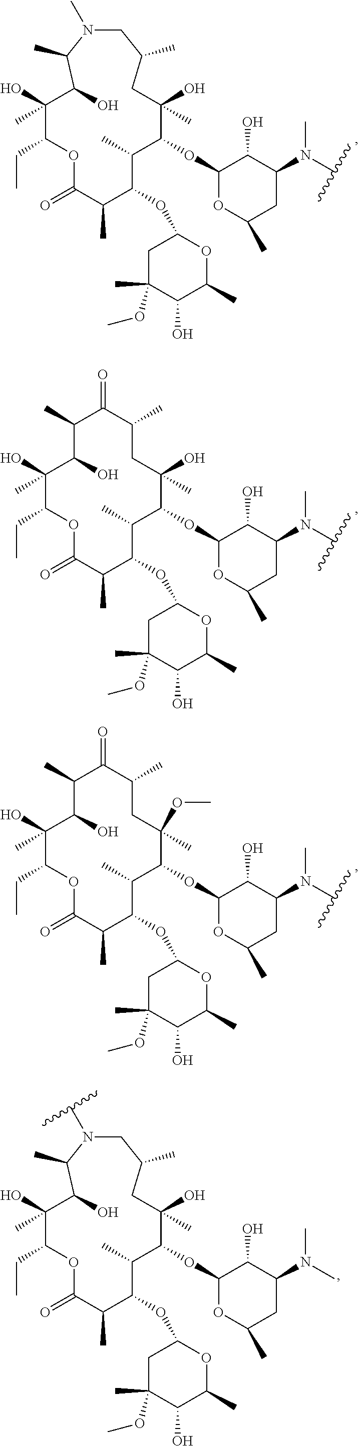

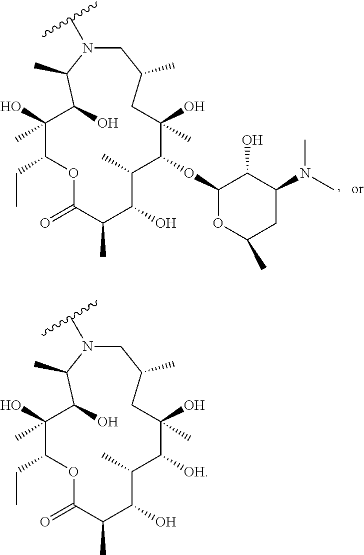 [SMILES: *N1C[C@H](C)C[C@@](C)(O)[C@H](O[C@@H]2O[C@H](C)C[C@H](N(C)C)[C@H]2O)[C@@H](C)[C@H](O)[C@@H](C)C(=O)O[C@H](CC)[C@@](C)(O)[C@H](O)[C@H]1C ; "," ; "or" ; *N1C[C@H](C)C[C@@](C)(O)[C@H](O)[C@@H](C)[C@H](O)[C@@H](C)C(=O)O[C@H](CC)[C@@](C)(O)[C@H](O)[C@H]1C]

In another aspect, the present invention provides fatty acid macrolide derivatives according to Formula Ia:

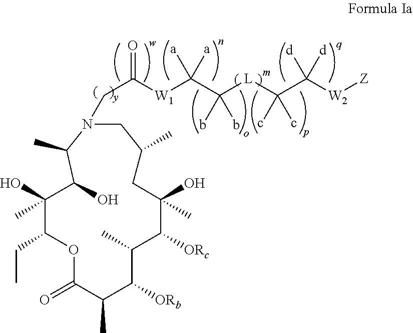

with the proviso that there is at least one

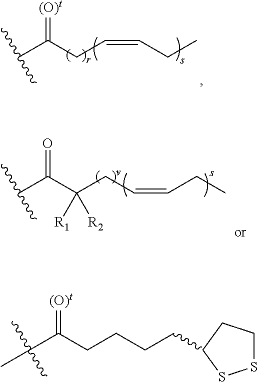

in the compound.

In some embodiments, R_{b} is

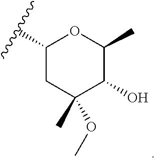 [SMILES: *[C@H]1C[C@@](C)(OC)[C@@H](O)[C@H](C)O1]

In some embodiments, R_{c} is

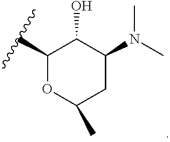 [SMILES: *[C@@H]1O[C@H](C)C[C@H](N(C)C)[C@H]1O]

In some embodiments,
R_{b is}

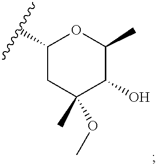 [SMILES: *[C@H]1C[C@@](C)(OC)[C@@H](O)[C@H](C)O1]

and
R_{c is}

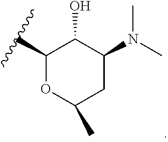 [SMILES: *[C@@H]1O[C@H](C)C[C@H](N(C)C)[C@H]1O]

In some embodiments, R_{b is H.}

In some embodiments, R_{b and R}_{c are each H.}

In another aspect, the present invention provides fatty acid macrolide derivatives according to Formula Ib:

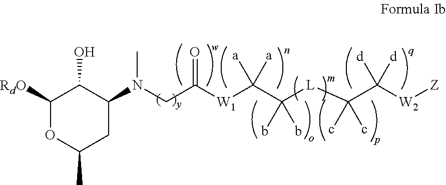

and pharmaceutically acceptable salts, hydrates, solvates, prodrugs, enantiomers, and stereoisomers thereof:

wherein

W₁, W₂, a, b, c, d, e, k, m, m1, n, o, p, q, I, Z, r, s, t, v, w, y, R_{b}, R_{d}, R₁, R₂, R₃, R₄, R and R₆ are as defined above for Formula Ib:

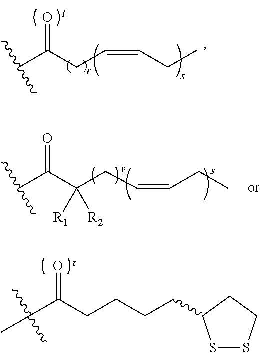

in the compound.

In some embodiments, R_{b} is

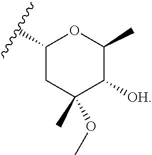 [SMILES: *[C@H]1C[C@@](C)(OC)[C@@H](O)[C@H](C)O1]

In some embodiments, R_{d} is

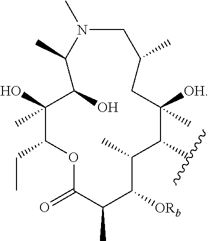

In some embodiments, R_{d} is

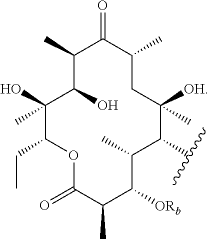

In some embodiments, R_{d} is

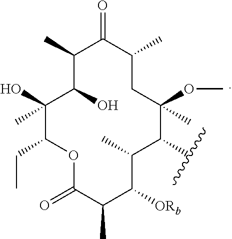

In some embodiments of Formula I, Ia, and Ib, one Z is

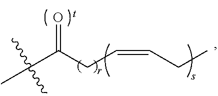

and r is 2.

In some embodiments of Formula I, Ia, and Ib, one Z is

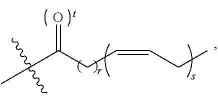

and r is 3.

In some embodiments of Formula I, Ia, and Ib, one Z is

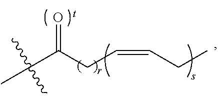

and r is 7.

In other embodiments of Formula I, Ia, and Ib, one Z is

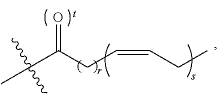

and s is 3.

In some embodiments of Formula I, Ia, and Ib, one Z is

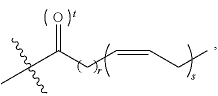

and s is 5.

In some embodiments of Formula I, Ia, and Ib, one Z is

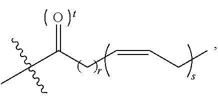

and s is 6.

In some embodiments of Formula I, Ia, and Ib, one Z is

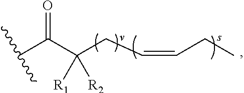

and v is 1.

In other embodiments of Formula I, Ia, and Ib, one Z is

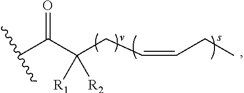

and v is 2.

In some embodiments of Formula I, Ia, and Ib, one Z is

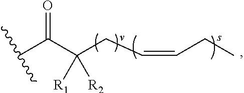

and v 6.

In some embodiments of Formula I, Ia, and Ib, one Z is

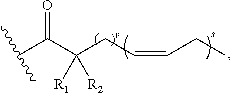

and is 3.

In some embodiments of Formula I, Ia, and Ib, one Z is

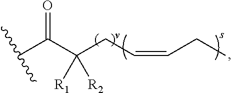

and s 5.

In some embodiments of Formula I, Ia, and Ib, one Z is

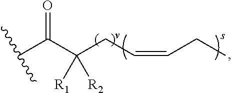

and s 6.

In other embodiments of Formula I, Ia, and Ib, one Z is

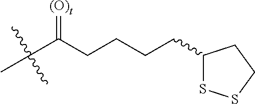

t is 1.

In some embodiments of Formula I, Ia, and Ib, Z is

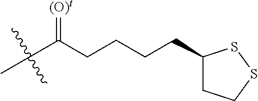

and t is 1.

In some embodiments of Formula I, Ia, and Ib, W₁ is null, O, NH, or N substituted with a C₁-C₆ alkyl.

In some embodiments of Formula I, Ia, and Ib, W₂ is null, O, NH, or N substituted with a C₁-C₆ alkyl.

In some embodiments of Formula I, Ia, and Ib, each a and c is independently H, Ch₃, —OCH₃, —OCH₂CH₃, or C(O)OR.

In some embodiments of Formula I, Ia, and Ib, w is 1.

In some embodiments of Formula I, Ia, and Ib, w is 0.

In some embodiments of Formula I, Ia, and Ib, m is 0.

In some embodiments of Formula I, Ia, and Ib, m is 1.

In some embodiments of Formula I, Ia, and Ib, m is 2.

In some embodiments of Formula I, Ia, and Ib, L is —S—, —S(O)—, —S(O)₂—, or —S-S—.

In some embodiments of Formula I, Ia, and Ib, L is —O—,

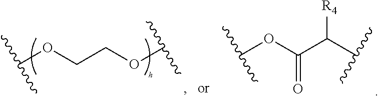

In some embodiments of Formula I, Ia, and Ib, L is

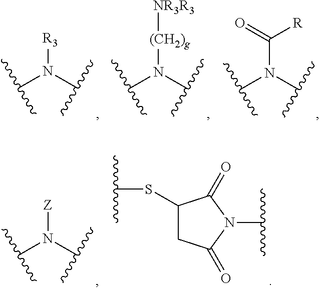

In some embodiments of Formula I, Ia, and Ib, L is

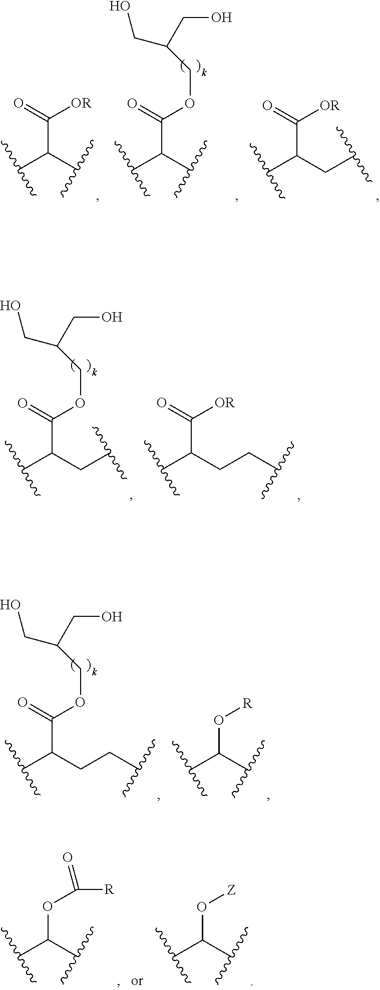

In some embodiments of Formula I, Ia, and Ib, L is

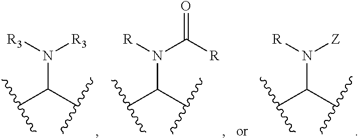

In some embodiments of Formula I, Ia, and Ib, L is

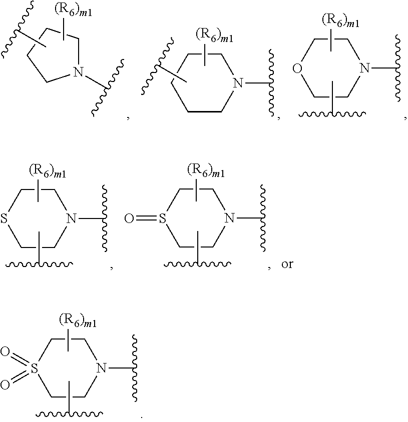

In some embodiments of Formula I, Ia, and Ib, L is

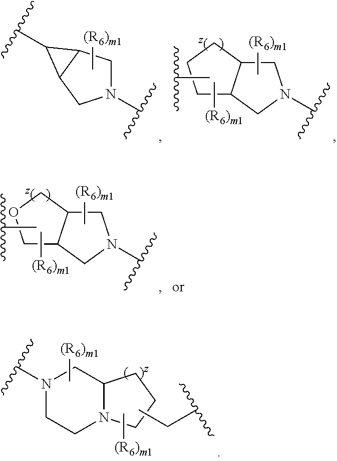

In some embodiments of Formula I, Ia, and Ib, L is

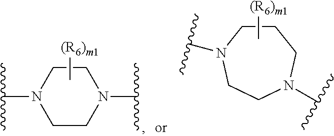

In some embodiments of Formula I, Ia, and Ib, L is

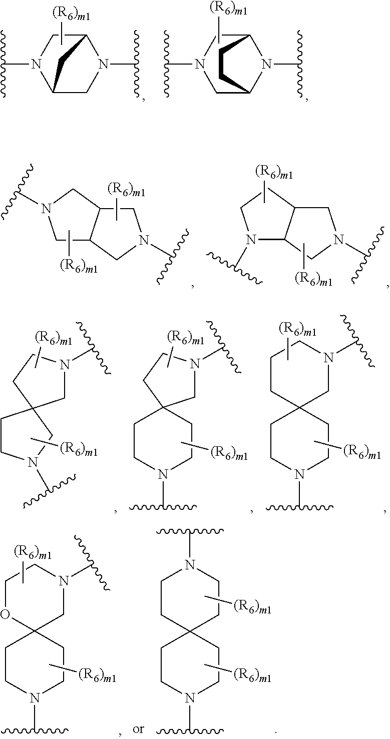

In some embodiments of Formula I, Ia, and Ib, one b is O-Z, Z is

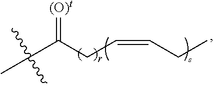

and t is 1.

In some embodiments of Formula I, Ia, and Ib, one d is C(O)OR.

In some embodiments of Formula I, Ia, and Ib, n, o, p, and q are each 1.

In some embodiments of Formula I, Ia, and Ib, two of n, o, p, and q are each 1.

In some embodiments of Formula I, Ia, and Ib, three of n, o, p, and q are each 1.

In some embodiments of Formula I, Ia, and Ib, t is 1.

In other illustrative embodiments, compounds of Formula Ia are as set forth below.
(b 2R,3S,4R,5R,8R,10R,11R,12S,13S,14R)-11-(((2S,3R,4S,6R)-4-(dimethylamino)-3- hydroxy-6-methyltetrahydro-2H-pyran-2-yl)oxy)-6-((4Z,7Z,10Z,13Z,16Z,19Z)-docosa- 4,7,10,13,16,19-hexaenoyl)-2-ethyl-3,4,10-trihydroxy-13-(((2R,4R,5S,6S)-5-hydroxy-4- methoxy-4,6-dimethylterahydro-2H-pyran-2-yl)oxy)-3,5,8,10,12,14-hexamethyl-1-oxa-6- azacyclopentadecan-15-one (Ia-1)
(2R,3S,4R,5R,8R,10R,11R,12S,13S,14R)-11-(((2S,3R,4S,6R)-4-dimethylamino)-3- hydroxy-6-methyltetrahydro-2H-pyran-2-yl)oxy)-6-((4Z,7Z,10Z,13Z,16Z,19Z)-docosa- 4,7,10,13,16,19-hexaenoyl)-2-ethyl-3,4,10,13-tetrahydroxy-3,5,8,10,12,14-hexamethyl-1- oxa-6-azacyclopentadecan-15-one (Ia-2)
(2R,3S,4R,5R,8R,10R,11R,12S,13S,14R)-6-((4Z,7Z,10Z,13Z,19Z)-docosa- 4,7,10,13,16,19-hexaenoyl)-2-ethyl-3,4,10,11,13-pentahydroxy-3,5,8,10,12,14-hexamethyl-1- oxa-6-azacyclopentadecan-15-one (Ia-3)
(2R,3S,4R,8,10R,11S,12S,13S,14R)-2-ethyl-3,4,10,11,13-pentahydroxy-6- ((5Z,8Z,11Z,14Z,17Z)-icosa-5,8,11,14,17-pentaenoyl)-3,5,8,10,12,14-hexamethyl-1-oxa-6- azacyclopentadecan-15-one (Ia-4)
(4Z,7Z,10Z,13Z,16Z,19Z)-N-(3-((2R,3S,4R,5R,8R,10R,11R,12S,13S,14R)-11- (((2S,3R,4S,6R)-4-(dimethylamino)-3-hydroxy-6-methyltetrahydro-2H-pyran-2-yl)oxy)-2- ethyl-3,4,10-trihydroxy-13-(((2R,4R,5S,6S)-5-hydroxy-4-methoxy-4,6-dimethyltetrahydro- 2H-pyran-2-yl)oxy)-3,5,8,10,12,14-hexamethyl-15-oxo-1-oxa-6-azacyclopentadecan-6-yl)-3- oxopropyl)docosa-4,7,10,13,16,19-hexaenamide (Ia-5)
(5Z,8Z,11Z,14Z,17Z)-N-((S)-1-((2R,3S,4R,5R,8R,10R,11R,12S,13S,14R)-11- (((2S,3R,4S,6R)-4-(dimethylamino)-3-hydroxy-6-methyltetrahydro-2H-pyran-2-yl)oxy)-2- ethyl-3,4,10,13-tetrahydroxy-3,5,8,10,12,14-hexamethyl-15-oxo-1-oxa-6- azacyclopentadecan-6-yl)-1-oxopropan-2-yl)icosa-5,8,11,14,17-pentaenamide (Ia-60
(4Z,7Z,10Z,13Z,16Z,19Z)-N-(3-((2R,3S,4R,5R,8R,10R,11R,12S,13S,14R)-11- (((2S,3R,4S,6R)-4-(dimethylamino)-3-hydroxy-6-methyltetrahydro-2H-pyran-2-yl)oxy)-2- ethyl-3,4,10-trihydroxy-13-(((2R,4R,5S,6S)-5-hydroxy-4-methoxy-4,6-dimethylietrahydro- 2H-pyran-2-yl)oxy)-3,5,8,10,12,14-hexamethyl-15-oxo-1-oxa-6-azacyclopentadecan-6- yl)propyl)docosa-4,7,10,13,16,19-hexaenamide (Ia-7)
(5Z,8Z,11Z,14Z,17Z)-N-(((2R,3S,4R,5R,10R,11R,12S,13S,14R)-11-(((2S,3R,4S,6R)- 4-(dimethylamino)-3-hydroxy-6-methyltetrahydro-2H-pyran-2-yl)oxy)-2-ethyl-3,4,10- trihydroxy-13-(((2R,4R,5S,6S)-5-hydroxy-4-methoxy-4,6-dimethyltetrahydro-2H-pyran-2- yl)oxy)-3,5,8,10,12,14-hexamethyl-15-oxo-1-oxa-6-azacyclopentadecan-6-yl)propyl)icosa- 5,8,11,14,17-pentaenamide (Ia-8)
(4Z,7Z,10Z,13Z,16Z,19Z)-N-(3-((2R,3S,4R,5R,8R,10R,11R,12S,13S,14R)-11- (((2S,3R,4S,6R)-4-(dimethylamino)-3-hydroxy-6-methyltetrahydro-2H-pyran-2-yl)oxy-2- ethyl-3,4,10,13-tetrahydroxy-3,5,8,10,12,14-hexamethyl-15-oxo-1-oxa-6- azacyclopentadecan-6-yl)propyl)docosa-4,7,10,13,16,19-hexaenamide (Ia-9)
(4Z,7Z,10Z,13Z,16Z,19Z)-N-(3-((2R,3S,4R,5R,8R,10R,11R,12S,13S,14R)-2-ethyl- 3,4,10,11,13-pentahydroxy-3,5,8,10,12,14-hexamethyl-15-oxo-1-oxa-6-azacyclopentadecan- 6-yl)propyl)docosa-4,7,10,13,16,19-hexaenamide (Ia-10)

In other illustrative embodiments, compounds of Formula Ib are as set forth below.
(4Z,7Z,10Z,13Z,16Z,19Z)-N-((2S,3R,4S,6R)-2-(((2R,3S,4R,5R,8R,10R,11R,12S,13S,14R)- 2-ethyl-3,4,10-trihydroxy-13-(((2R,4R,5S,6S)-5-hydroxy-4-methoxy-4,6-dimethyltetrahydro- 2H-pyran-2-yl)oxy)-3,5,6,8,10,12,14-heptamethyl-15-oxo-1-oxa-6-azacyclopentadecan-11- yl)oxy)-3-hydroxy-6-methyltetrahydro-2H-pyran-4-yl)-N-methyldocosa-4,7,10,13,16,19- hexaenamide (Ib-1)
(5Z,8Z,11Z,14Z,17Z)-N-((2S,3R,4S,6R)-2-(((2R,3S,4R,5R,8R,10R,11R,12S,13S,14R)-2- ethyl-3,4,10-trihydroxy-13-(((2R,4R,5S,6S)-5-hydroxy-4-methoxy-4,6-dimethyltetrahydro- 2H-pyran-2-yl)oxy)-3,5,6,8,10,12,14-heptamethyl-15-oxo-1-oxa-6-azacyclopentadecan-11- yl)oxy)-3-hydroxy-6-methyltetrahydro-2H-pyran-4-yl)-N-methylicosa-5,8,11,14,17- pentaenamide (Ib-2)
(4Z,7Z,10Z,13Z,16Z,19Z)-N-((S)-1-(((2S,3R,4S,6R)-2- (((2R,3S,4R,5R,8R,10R,11R,12S,13S,14R)-2-ethyl-3,4,10-trihydroxy-13-(((2R,4R,5S,6S)-5- hydroxy-4-methoxy-4,6-dimethyltetrahydro-2H-pyran-2-yl)oxy)-3,5,6,8,10,12,14- heptamethyl-15-oxo-1-oxa-6-azacyclopentadecan-11-yl)oxy)-3-hydroxy-6-methyltetrahydro- 2H-pyran-4-yl)(methyl)amino)-1-oxopropan-2-yl)docosa-4,7,10,13,16,19-hexaenamide (Ib-3)
(4Z,7Z,10Z,13Z,16Z,19Z)-N-(((2R,3S,4R,5R,8R,10R,11R,12S,13S,14R)- 2-ethyl-3,4,10,13-tetrahydroxy-3,5,6,8,10,12,14-heptamethyl-15-oxo-1-oxa-6- azacyclopentadecan-11-yl)oxy)-3-hydroxy-6-methyltetrahydro-2H-pyran-4-yl)-N- methyldocosa-4,7,10,13,16,19-hexaenamide (Ib-4)
(5Z,8Z,11Z,14Z,17Z)-N-((2S,3R,4S,6R)-2-(((2R,3S,4R,5R,8R,10R,11R,12S,13S,14R)-2- ethyl-3,4,10,13-tetrahydroxy-3,5,6,8,10,12,14-heptamethyl-15-oxo-1-oxa-6- azacyclopentadecan-11-yl)oxy)-3-hydroxy-6-methyltetrahydro-2H-pyran-4-yl)-N- methylicosa-5,8,11,14,17-pentaenamide (Ib-5)

Methods for Using Fatty Acid Macrolide Derivatives

Also provided in the invention is a method for inhibiting, preventing, or treating inflammation or an inflammatory disease in a subject. The inflammation can be associated with an inflammatory disease or a disease where inflammation contributes to the disease. Inflammatory diseases can arise where there is an inflammation of the body tissue. These include local inflammatory responses an systemic inflammation. Examples of such diseases include, but are not limited to: organ transplant rejection; reoxygenation injury resulting from organ transplantation (Grupp et al. J. Mol. Cell. Cardiol. 1999, 31, 297-303) including, but not limited to, transplantation of the following organs: heart, lung, liver and kidney; chronic inflammatory diseases of the joints, including arthritis, rheumatoid arthritis, osteoarthritis and bone diseases associated with increased bone resportion; inflammatory bowel diseases such as ileitis, ulcerative colitis, Barrett's syndrome, and Crohn's disease; inflammatory lung diseases such as asthma, adult respiratory distress syndrome, chronic obstructive airway disease, and cystic fibrosis; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; chronic kidney disease (CKD); IgA nephropathy; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalmoyelitis, Parkinson's disease, Huntington's disease, amyotrophic lateral sclerosis and viral or autoimmune encephalitis. Metabolic disease such as type II diabetes mellitus; the prevention of type I diabetes; dyslipidemia, hypertriglyceridemia; diabetic complications, including, but not limited to glaucoma, retinopathy, macula edema, nephropathy, such as microalbuminuria and progressive diabetic nephropathy, polyneuropathy, diabetic neuropathy, atherosclerotic coronary arterial disease, peripheral arterial disease, nonketotic hyperglycemic hyperosmolar coma, mononeuropathies, autonomic neuropathy, joint problems, and a skin or mucous membrane complication, such as an infection, a shin spot, a candidal infection or necrobiosis lipoidiea diabeticorum; immune-complex vasculitis, systemic lupus erythematosus; inflammatory diseases of the heart such as cardiomyopathy, ischemic heart disease hypercholesterolemia, and atherosclerosis; as well as various diseases that can have significant inflammatory components, including preeclampsia; chronic liver failure, brain an spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to proinflammatory cytokines, e.g., shock associated with proinflammatory cytokines. Such shock can be induced, e.g., by a chemotherapeutic agent that is administered as a treatment for cancer. Other disorders include depression, obesity, allergic diseases, acute cardiovascular events, arrhythmia, prevention of sudden death, muscle wasting diseases such as Duchenne's Muscular Dystrophy, inflammatory myopathies such as dermatomositis, inclusion body myositis, and polymyositis, and cancer cachexia. Inflammation that results from surgery and trauma can also be treated with a fatty acid macrolide derivative.

In some embodiments, the subject is administered an effective amount of a fatty acid macrolide derivative.

Effective dosage amounts of the present invention, when used for the indicated effects, range from about 20 mg to about 5,000 mg of the fatty acid macrolide derivative per day. Compositions for in vivo or in vitro use can contain about 20, 50, 75, 100, 150, 250, 500, 750, 1,000, 1,250, 2,500, 3,500, or 5,000 mg of the fatty acid macrolide derivative. In one embodiment, the compositions are in the form of a tablet that can be scored. Effective plasma levels of the fatty acid macrolide derivative can range from about 5 ng/mL to 5000 ng/mL. Appropriate dosages of the fatty acid macrolide derivatives can be determined as set forth Goodman, L. S.; Gilman, A. The Pharmacological Basis of Therapeutics, 5th ed.; MacMillan: New York, 1975, pp. 201-226.

The invention also includes pharmaceutical compositions useful for treating or preventing a metabolic disorder, or for inhibiting a metabolic disorder, or more than one of these activities. The compositions can be suitable for internal use and comprise an effective amount of a fatty acid macrolide derivative and a pharmaceutically acceptable carrier. The fatty acid macrolide derivatives are especially useful in that they demonstrate very low peripheral toxicity or no peripheral toxicity.

Administration of the fatty acid macrolide derivatives can be accomplished via any mode of administration for therapeutic agents. These modes include systemic or local administration such as oral, nasal, parental, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a fatty acid macrolide derivative and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmol MCM, capmol PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the fatty acid macrolide derivative is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the fatty acid macrolide derivatives.

The fatty acid macrolide derivatives can be also formulated as a suppository that can be prepared from fatty emulsions or suspensions; using polyalkylene glycols, such as propylene glycol, as the carrier.

The fatty acid macrolide derivatives can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylscholines. In some embodiments, a film of lipid components is hydrated with an aqueous solution of drug to a form lipid layer encapsulating the drug, as described in U.S. Pat. No. 5,262,564, the contents of which are hereby incorporated by reference.

Fatty acid macrolide derivatives can also be delivered by the use of monoclonal antibodies as individual carriers to which the fatty acid macrolide derivatives are coupled. The fatty acid macrolide derivatives can also be coupled with soluble polymers as targetable drug carriers. Such polymers can include polyvinylpyrrolidine, pyran copolymer, polyhydroxypropylmethacrylamide-phenol, polyhydroxyethylaspanamidephenol, or polyethyleneoxidepolylysine substituted with palmitoyl residues. Furthermore, the fatty acid macrolide derivatives can be coupled to a class of biodegradeable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, fatty acid macrolide derivatives are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 90%, from about 10% to about 90%, or from about 30% to about 90 % of the fatty acid macrolide derivative by weight or volume.

The degree regimen utilizing fatty acid macrolide derivatives is selected in accordance with a variety of factors including type, species, age, weight, sex and medical condition of the patient; the severity of the condition to be treated; the route of administration; the renal or hepatic function of the patient; and the particular fatty acid macrolide derivative employed. A physician or veterinarian of ordinary skill in the art can readily determine and prescribe the effective amount of the drug required to prevent, counter or arrest the progress of the condition.

Fatty acid macrolide derivatives can be administered in a single daily dose, or the total daily dosage can be administered in divided doses of two, three or four times daily. Furthermore, fatty acid macrolide derivatives can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the fatty acid macrolide derivative ranges from about 0.1% to about 15%, w/w or w/v.

Methods for Making the Fatty Acid Macrolide Derivatives

Examples of synthetic pathways useful for making fatty acid macrolide derivatives of Formula I, Formula Ia and Formula Ib are set forth in the Examples below and generalized in Schemes 1-11.

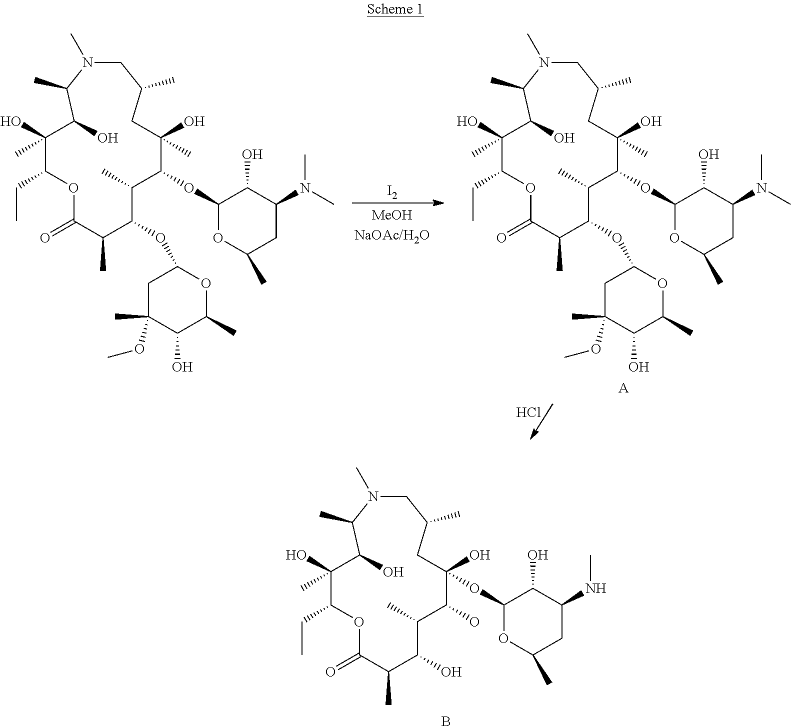

Azithromycin can be converted to the 3′-N-desmethyl azithromycin derivative A by treatment with I₂ in MeOH containing aqueous. NaOAc according to the procedure outlined in Oyclere et al. J. Med. Chem. 2009, 52, 456-468. One skilled in the art will recognize that the cladinose moiety in A can be removed by treatment with an acid such as HCl to afford B. One skilled in the art will also recognize that the chemistry shown in Scheme 1 can be repeated with erythromycin, clarithromycin and roxithromycin in order to remove one methyl group in the desosamine moiety.

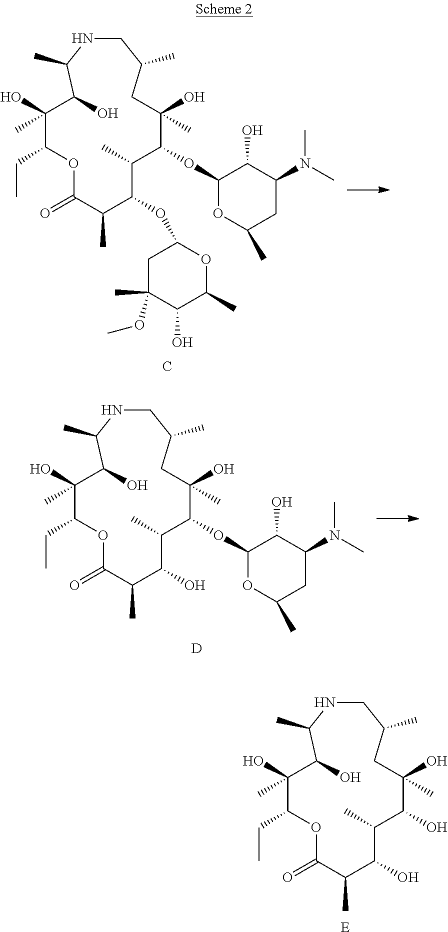

The 9-a-N-desmethyl azithromycin derivative C is a well-known precursor to azithromycin and can be obtained from the standard procedures outlined in U.S. Pat. No. 4,517,357 and International Application No. PCT/US2001/000364. Upon treatment with dilute acids such as HCl, the cladinose moiety can be removed according to the procedure outlined in Oyelere et al. J. Med. Chem. 2009, 52, 456-468. Upon further treatment with acids over an extended period, both the cladinose and desosamine moieties can be removed. One skilled in the art will recognize that this type of chemistry can be repeated on other derivatives of azithromycin to remove the cladinose moiety, or both the desosamine and cladinose moieties.

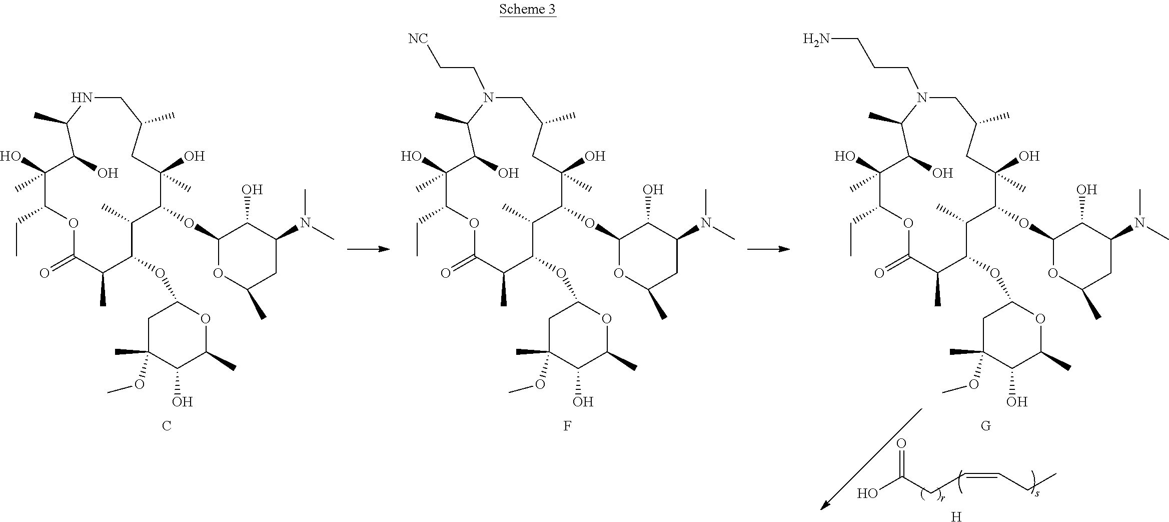

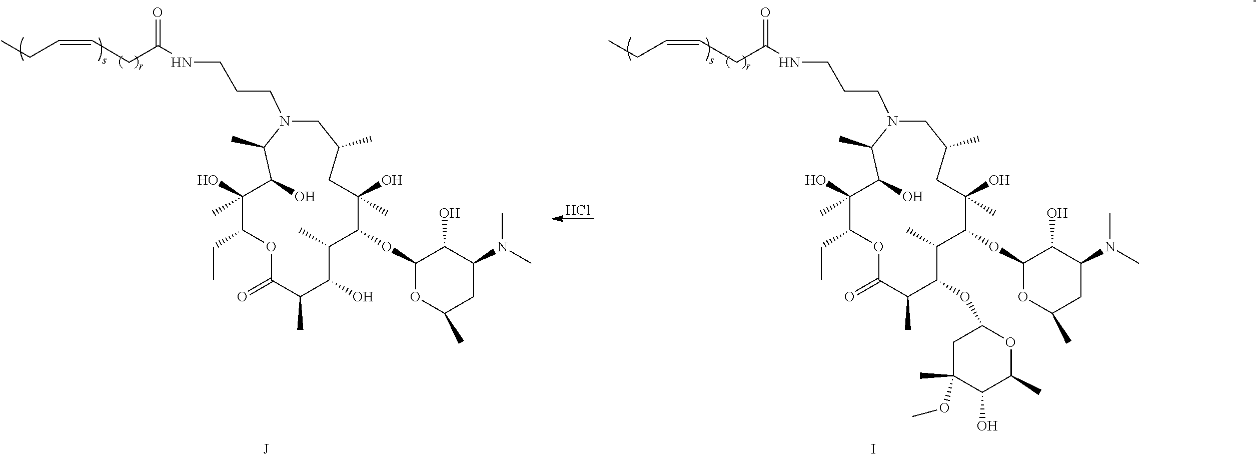

wherein r and s are defined above.

Compound C can be reacted with acrylonitrile according to the procedures outlined in International Application No. PCT/IB2005/003213 to obtain the nitrile derivative F. The nitrile group can then be reduced to the corresponding amine derivative G by hydrogenation over platinum dioxide. Compound G can be coupled with a fatty acid of the formula H using HATU in the presence of a base such as DIEA to afford compounds of the formula I. To those familiar in the art, the fatty acid H can also be substituted with lipoic acid in this scheme and in the subsequent schemes. One skilled in the art will recognize that the cladinose moiety in compounds of the formula I can be removed to obtain compounds of the formula J by treatment with an acid, such as HCl. One skilled in the art will also recognize that compounds D and E can be used in place of 9a-N-dosmethyl azithromycin C in order to prepare the corresponding analogs lacking the respective desosamine and/or cladinose moieties.

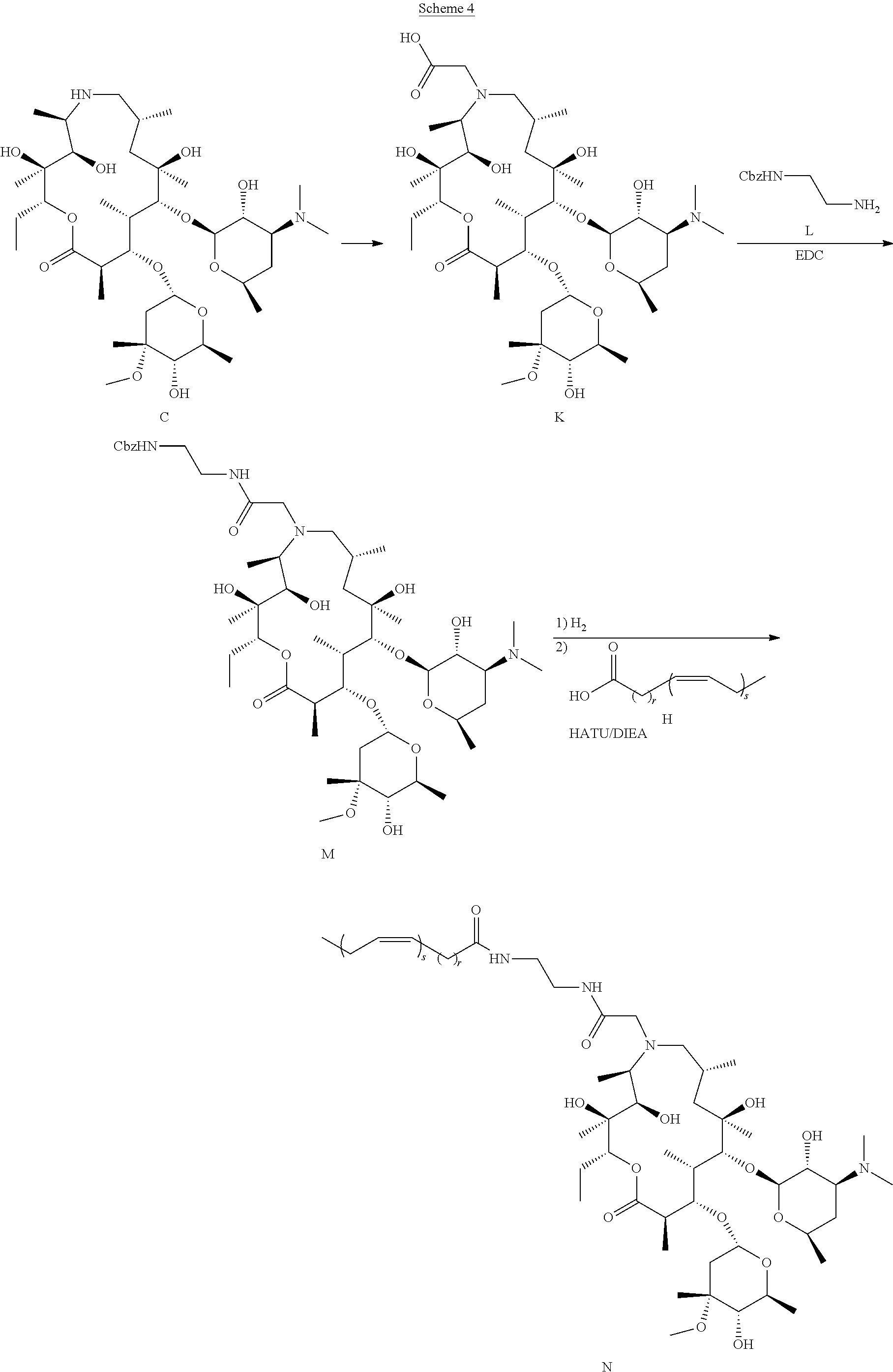

wherein r and s are as defined above.

Compound C can be reacted with benzyl bromoacetate, followed by hydrogenation over palladium on carbon to afford intermediate K. The intermediate acid K can be coupled with the mono-Cbz protected amine L using either EDC or HATU to afford intermediate M. Compounds M can be hydrogenerated over palladium on carbon to remove the Cbz protecting group. The resulting amine can be reacted with a fatty acid of the formula H using HATU in the presence of an amine such as DIEA to afford compounds of the formula N.

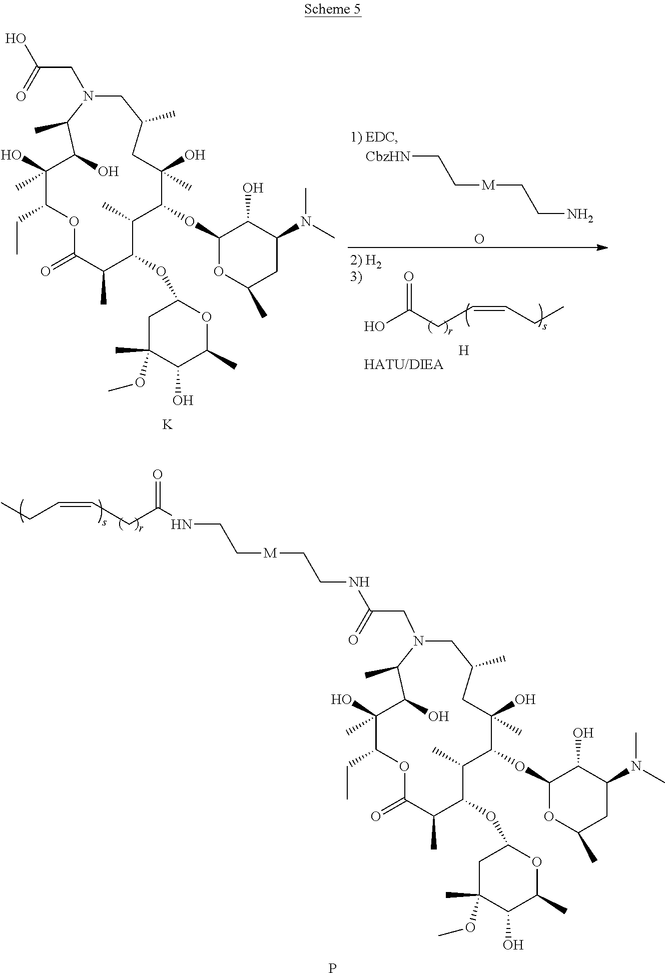

wherein M is —O—, —S—, —S-S—, —CH(OH)—, —OCH₂CH₂O—, —NR-, or —C(O)NR-, and R, r, and s are a defined above.

The intermediate K can be reacted with an amine of the formula O using a reaction sequence similar to that shown in Scheme 4 to obtain compounds of the formula P. The mono-Cbz protected amine of the Formula O (wherein M is —NR-) can be obtained from commercial sources or prepared according to the procedures outlined in Krapcho et al. Synthetic Commun. 1990, 20, 2559-2564. The acylated amine of the Formula O (wherein M is —C(O)NR-) can be prepared using the procedures outlined in Andruszkiewicz et al. Synthetic Commun. 2008, 38, 905-913. The amine O (wherein M is O) can be prepared according to the procedures outlined in Dahan et al. J. Org. Chem. 2007, 72, 2289-2296. The amine O (wherein M is —CH(OH)—, —S—, or —OCH₂CH₂O—) can be obtained from commercial sources. The amine O (wherein M is —S-S—) can be prepared according to the procedures outlined in Jacobson, K. et al. Bioconjugate Chem. 1995, 6, 255-263.

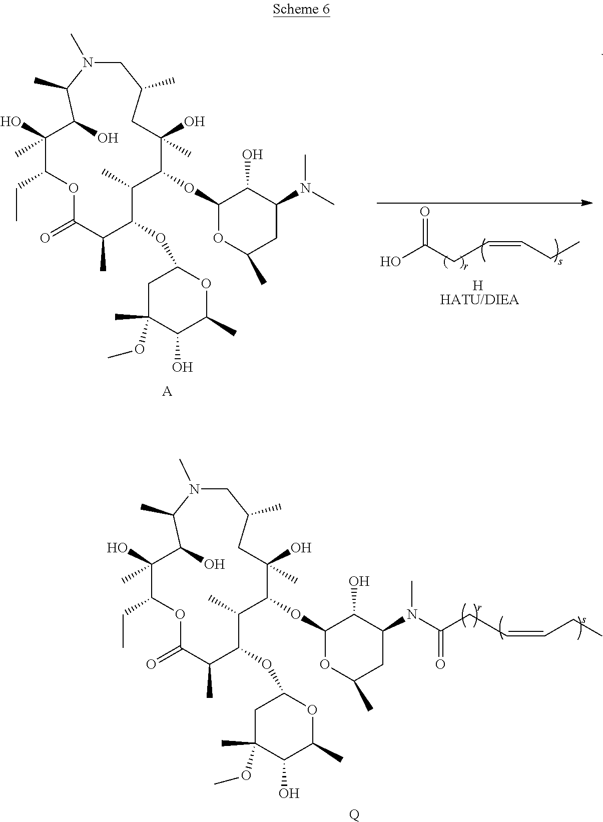

wherein r and s are defined above.

Compound A can be coupled with a fatty acid of the Formula F using HATU in the presence of an amine such as DIEA to afford compounds of the formula O. One skilled in the art will recognize that the synthetic sequence outlined in Scheme 6 can be performed with compound B in place of compound A to prepare compounds lacking the cladinose moiety.

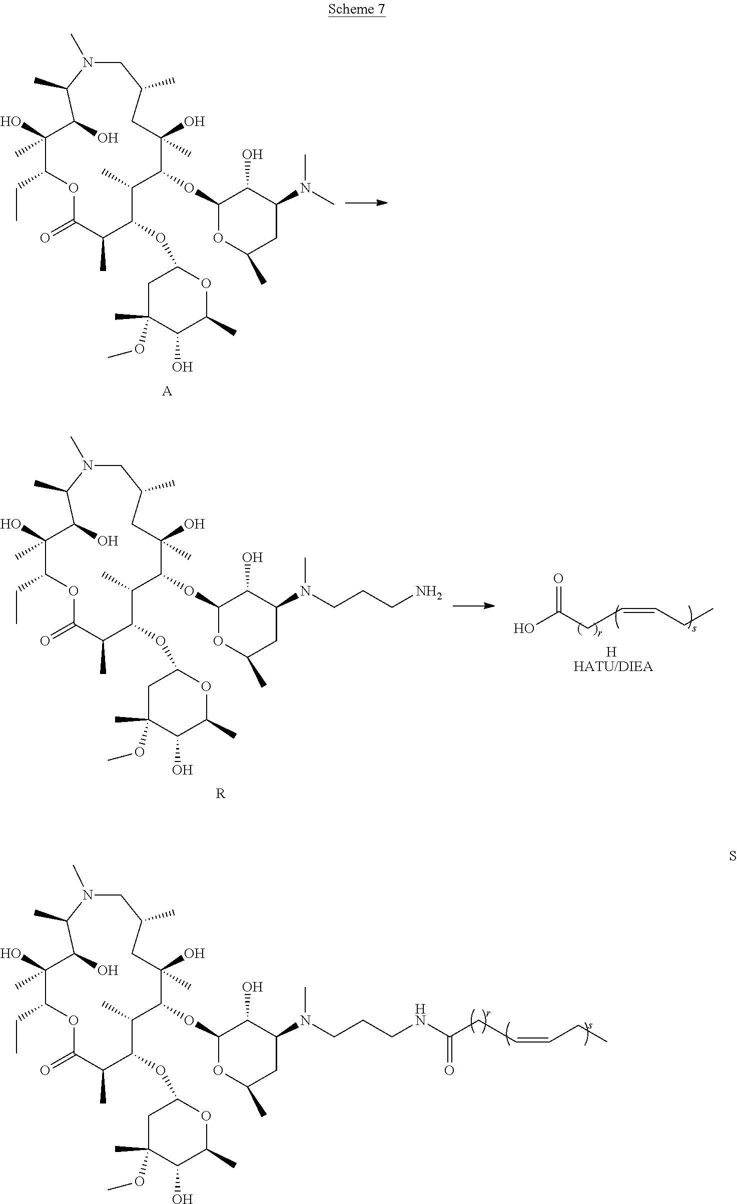

wherein r and s are as defined above.

Compound A can be subjected to the same procedures outlined in Scheme 3 and in International Application No. PCT/IB2005/003213 to obtain the intermediate nitrile, which in turn can be reduced to the corresponding amine derivative R by hydrogenation over platinum dioxide. Compound R can then be coupled with a fatty acid of formula H using HATU in the presence of DIEA to obtain compounds of the formula S. One skilled in the art will recognize that the synthetic sequence outlined in Scheme 7 can be repeated with compound B in place of compound A to prepare compounds lacking the cladinose moiety.

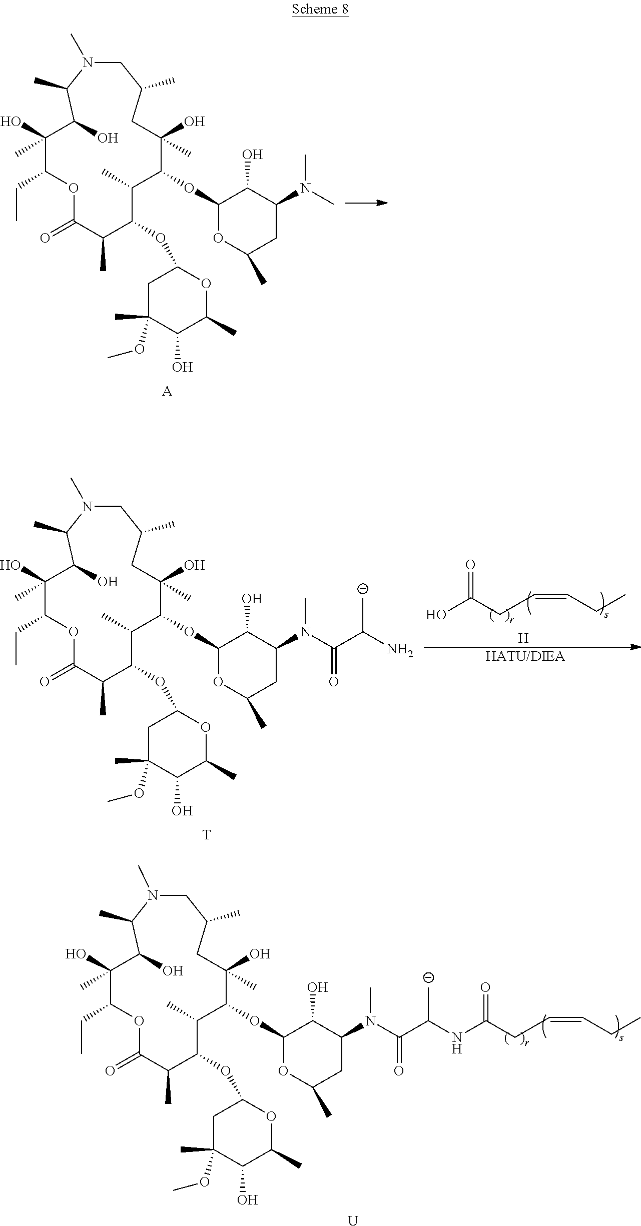

where e, r, and s are as defined above.

Compound A is coupled with the Cbz-protected amino acid using EDCI, followed by hydrogenation over palladium on carbon to produce the intermediate amine T. Compound T can then be coupled with a fatty acid of formula H using HATU in the presence of DIEA to afford compounds of the formula U.

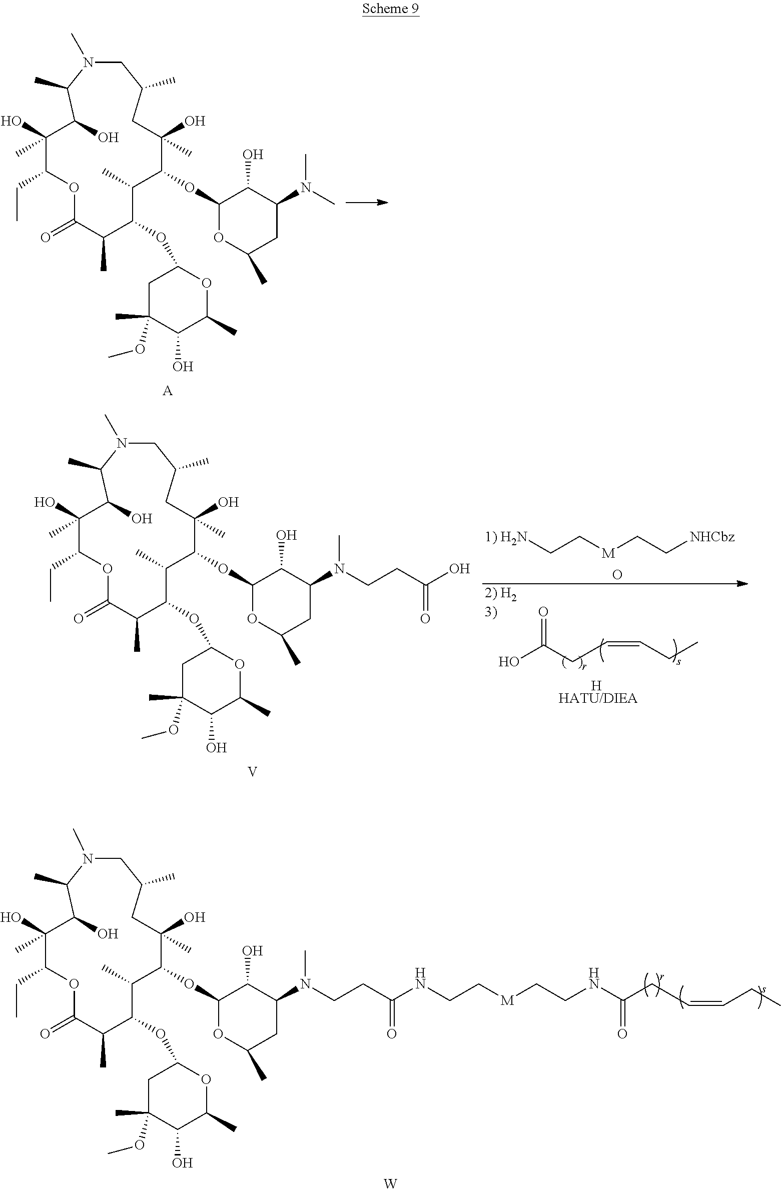

wherein M is —O—, —S—, —S-S—, —CH(OH)—, —OCH₂CH₂O—, —NR-, or —C(O)NR-, and R, r, and s are as defined above.

Compound A can be reacted with benzyl acrylate, followed by hydrogenation over palladium on carbon to afford compound V. Compound V can then be coupled with an amine of the formula O. The resuling intermediate can be hydrogenated over palladium on carbon and then coupled with a fatty acid of the formula H using HATU in the presence of DIEA to obtain compounds of the formula W. The mono-Cbz protected amine of the Formula O (wherein M is —NR—) can be obtained from commercial sources or prepared according to the procedures outlined in Krapcho et al. Synthetic Commun. 1990, 20, 2559-2564. The acylated amine of the Formula O (wherein M is —C(O)NR—) can be prepared using the procedures outlined in Andruszkiewicz et al. Synthetic Commun. 2008, 38, 905-913. The amine O (wherein M is —O—) can be prepared according to the procedures outlined in Dahan et al. J. Org. Chem. 2007, 72, 2289-2296. The amine O (wherein M is —(CH(OH)—, —S-, or —OCH₂CH₂O—) can be obtained from commercial sources. The amine O (wherein M is —S-S—) can be prepared according to the procedures outlined in Jacobson, K. et al. Bioconjugate Chem. 1995, 6, 255-263.

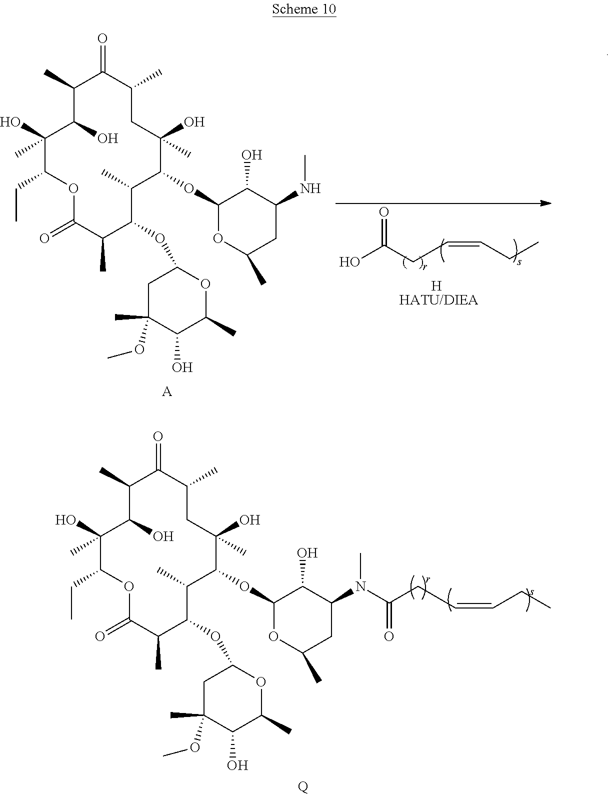

wherein R, r, and s are defined above.

Compound X (wherein R is H) can be obtained from erythromycin by using the sequence outlined in Oyelere et al. J. Med. Chem. 2009, 52, p. 456-468. Compound X (wherein R is CH₃) can be obtained from clarithromycin by using the sequence outlined in Oyelere et al. J. Med. Chem. 2009, 52, p. 456-468. Compound X can be coupled with a fatty acid of the formula H using HATU in the presence of DIEA to obtain compounds of the formula Y.

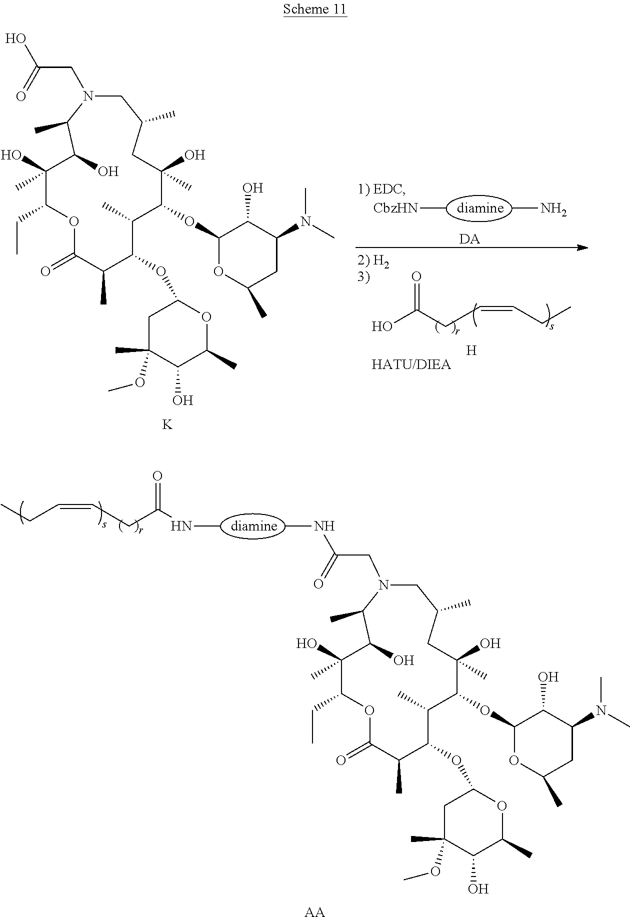

wherein R, r, and s are as defined above.

Compound K can be coupled with a Cbz-protected diamine of the general formula DA to obtain the BOC-protected amide derivative. After removal of the Cbz protecting group by standard hydrogenation, the resulting amine can be coupled with a fatty acid of the formula H in order to obtain compounds of the formula AA. A variety of Cbz-protected diamines are commercially available. The following diamines can be prepared according to the procedures outlined in the corresponding references:

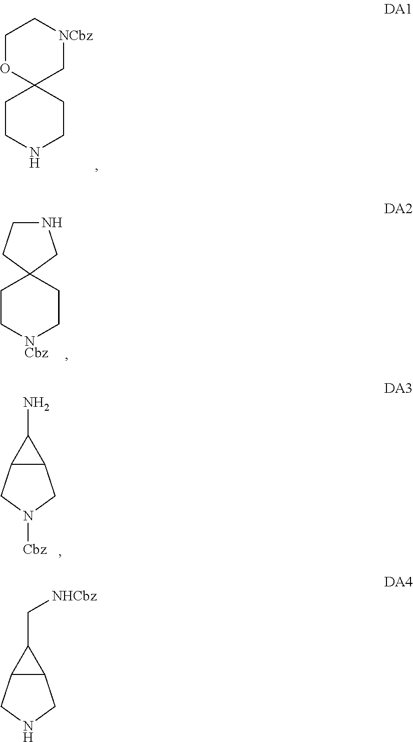 [SMILES: O=C(OCc1ccccc1)N1CCOC2(CCNCC2)C1 ; O=C(OCc1ccccc1)N1CCC2(CCNC2)CC1 ; NC1C2CN(C(=O)OCc3ccccc3)CC12 ; O=C(NCC1C2CNCC21)OCc1ccccc1]

diamine DA1, Stocks et al, Bioorganic and Medicinal Chemistry Letters 2010, p. 7458; diamine DA2, Fritch et al, Bioorganic and Medicinal Chemistry Letters 2010, p. 6375; diamine DA3 and DA4, Moffat et al, J. Med. Chem. 2010, 53, p.8663-8678). To those familiar in the art, detailed procedures to prepare a variety of mono-protected diamines can also be found in the following references: WO 2004092172, WO2004092171, and WO 2004092173.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Effects of Compounds of the Invention of NFκB Levels in RAW 264.7 Macrophages

RAW 264.7 cells stably expressing a 3× NFκB response element-drive luciferase reporter were seeded into 96 well plates in sera-free medium (Optimen) 18 hours prior to compound application. Compounds of the invention were prepared by first making 100 mM stock solutions in EtOH. Stock solutions were then diluted 1:100 in low LPS FBS (Gemini BenchMark 100-106), mixed vigorously and allowed to incubate at room temperature for 30 minutes. 1:2 serial dilutions were then made in FBS supplemented with 1% EtOH, mixed vigorously, and again allowed to incubate at room temperature for 30 minutes before adding to RAW 264.7 reporter cells (final concentrations: 10% FBS, 100 uM highest compound dilution, 0.1% EtOH) for a 2 hour pretreatment prior to stimulation with LPS. Cells were then stimulated with 200 ng/ml LPS or vehicle control for 3 hours in the presence of the compounds of the invention. A set of six vehicles was left unstimulated with LPS in order to measure the assay floor. AlamarBlue viability dye (Invitrogen) was added to cells simultaneously with the delivery of LPS (final AlamarBlue concentration of 10%). After the 3 h incubation period with LPS, cell viability was measured by reading fluorescence (excitation 550 nm, emission 595 nm) with a Perkin Elmer Victor V plate reader. Then cell media was aspirated from each well. Luciferase signal was then developed by addition of the Britelite Plus reagent (Perkin Elmer). Luciferase activity was measured with the Perkin Elmer Victor V plate reader. NF-κB activity was expressed as a percent of the vehicle control wells (stimulated with LPS). Compounds were tested at 6 dose point titrations in triplicate to determine IC₅₀ values.

As an illustrative example, FIG. 1 shows the effect of compound Ia-3 in this NF-κB reporter assay. The corresponding IC₅₀ was determined to be 18 uM. In this figure AB refers to Alamar Blue and FF refers to the luciferase activity.

### Example 2

### Effect of Fatty Acid Macrolide Derivatives on IL-1β, HMOX-1 and TNF-α

RAW264.7 macrophages are seeded at a density of 100,000 cells/well in a 96-well plate in DMEM supplemented with 10% FBS and Penn/strep. 16 hours later, medium is aspirated and replaced with 90 μL/well of serum-free DMEM. Compounds of the invention are brought up in 100% EtOH to a concentration of 100 mM and then diluted 1:100 in 100% FBS for a stock solution consisting of 1 mM compound and 1% EtOH. These stock solutions are then diluted 1:10 in FBS supplemented with 1% EtOH to generate a 100 μM of the fatty acid macrolide conjugate. 10 μL is then added to the RAW246.7 cells to generate final concentrations 10 μM of the fatty acid macrolide conjugate. The compounds are allowed to pre-incubate for 2 hours before stimulation of 100 mg/ml LPS (10 μL of 1 μg/ml LPS was added to each well). Following 3 hours of LPS stimulation, cells are washed once in 1× PBS, aspirated dry, and flash frozen in liquid nitrogen. RNA is then isolated and converted to cDNA using the Cells to cDNA kit (Ambion) according to the manufacturer's protocol IL-1β, HMOX-1 and TNF-α transcript levels are then measured using Taqman primer/probe assay sets (Applied Biosystems), normalized to GAPDH using the deltaCt method, and the data expressed relative to vehicle only control.

### Example 3

### TNFα Release Assay in RAW 264.7 Macrophages

The purpose of this assay is to measure the ability of small molecules to inhibit the secretion of TNFα in cultured macrophages stimulated with lipopolysaccharide (LPS). Treatment of macrophages with LPS activates inflammatory cytokine pathways primarily through the TLR4-NFκB signaling axis. Compounds of the invention inhibit the transcriptional activation of NFκB and thus decrease the production and release of TNFα. Dexamethasone, a potent agonist of the glucocorticoid receptor is used a positive control for inhibition of TNFα release.

Day 1L : Seed RAW 264.7 macrophages into 96 well culture plates. Remove culture media from RAW 264.7 cell growing in a 75 mm² tissue culture flask (cells should be at ˜70% confluence) and add 10 mL of warmed complete growth media (DMEM+10% FBS +1× pen/step). The cells are scraped into suspension using a sterile plate scraper and homogenized by pipetting up and down with a 10 mL serological pipette. The cell concentration is determined using a clinical hematoctyometer. Cells are then diluted to 150,000 cells per mL into growth media. The diluted cells are then transferred to a sterile reagent reservoir and 100 μl of cell suspension is pipetted into each well of a 96 well culture plate using a multichannel pipette (15,000 cells/well). Plates are then incubated at 37° C. under normal tissue culture growth conditions (37° C., humidified CO₂ chamber).

Day 2: The test compound sample plate is prepared. Test compounds are prepared in growth media. Compounds are delivered to media from 1000× stocks in 100% DMSO (e.g. for a 10 μM final concentration of test compound, deliver 2 μl of 10 mM test compound to 2 mL of media). At least 150 μl of 1× compound in media is added to 96 well sample plate. The perimeter wells of the 96 well plate are not used to avoid edge effect. Twelve sample wells are prepared with media plus 0.1% DMSO (these samples will serve as the vehicle controls. LPS-stimulated and non-stimulated; 10 μM dexamethasone is used as a positive control). Culture plates are then returned to the growth incubator for 2 hours. Cells are stimulated afterwards by adding 25 μl of 50 ng/mL LPS is added to every well (except the 6 unstimulated vehicle control wells: final concentration of 10 ng/mL LPS. Plates are returned to growth incubator for 3 hours. Afterwards, 100 μl of media supernatant is removed and transferred to a 96 well v-bottom sample plate. The media supernatant plate is centrifuged for 5 minutes at 1,000 rpm in a swing-bucket centrifuge, pelleting any cellular debris that may remain in supernatant. 80 μl of supernatant is removed from sample plate and transferred to a fresh v-bottom 96 well plate. Cell viability is measured using Celltiter-glo kit. By measuring cell viability, a given compound's effects on TNFα secretion can determine whether effects are due to cytotoxicity or to true inhibition of inflammatory signaling. Add 100 μl of Celltiter-glo reagent to each well of the cell culture plate and afterwards measure the luminescence signal (CPS) of the plate using the Victor 5 plate reader (0.3 second read; 60 second plate shaking prior to read). Cell viability of a given compound at a given concentration is computed as follows:

Cell viability=CPS Sample/(Average CPS unstimulated controls)*100

Use 20 μl of media supernatant per well for TNFα ELISA. Follow Invitrogen/Biosource manufacture's protocol for the mouse TNFα ELISA. Chromogen development is typically conducted for 20-30 minutes as described in the manufacture's protocol. After addition of stop solution, measure OD 450 nm using the Victor 5 plate reader (0.1 second/well scan). Determine the TNFα secretion percent of control. The following formula is used to determine the TNFα secretion percent of control:

$$\frac{\begin{matrix}100 \times ( OD 450 nm Sample X ) - \\ ( Average OD 450 nm unstimulated vehicle controls )\end{matrix}}{\begin{matrix}( Average OD 450 nm LPS stimulated vehicle controls ) - \\ ( Average OD 450 nm unstimulated vehicle controls )\end{matrix}}$$

For each test compound, TNFα secretion percent of control can be plotted as a function of compound concentration using a four parameter dose-response curve fit equation (XLFIT Model#205):

fit=(A+((B−A)/(1+((C/x)̂D))))

inv=(C/((((B−A)/(y−A))−1)̂(1D)))

res=(y−fit)

### Example 4

### In vivo Effects of Compounds of the Invention in a LPS-challenge TNFα Mouse Model

To measure the effects of compounds on TNFα secretion in vivo, Male Swiss Webster mice (n=10 animals per group) are dosed by either oral gavage or by ip injection with each test compound (dosing volume is 15 mL/kg). All compounds are formulated in the appropriate vehicles (Examples of vehicles that can be used include combinations of solvents such as polyethylene glycol and propyleneglycol, lipids such as glycerol monooleate and soybean oil, and surfactants such as polysorbate 80 and cremophor EL). Ninety minutes after compound dosing, animals are treated with 0.2 mg/kg LPS (lipopolysaccharide) by intraperitoneal (IP) injection. Ninety minutes after LPS challenge, mice are anesthetized and bled by cardiac puncture into serum separator tubes (with sodium heparin). Bleeds are allowed to clot at room temperature for 2 hours, and tubes are then spun for 20 minutes at 2,000 ×g. Serum is harvested from tubes (100-150 μl per animal) and frozen at −70° C. TNFα serum levels are measured using commercially available TNFα ELISA kits (*p<0.05 using a 2-tailed t-test). Dexamethasone (dosed at 0.5 mg/kg po) can be used as the positive control in the type of experiment.

### Example 5

### In vivo Effects of Compounds of the Invention in Murine Models of Cystic Fibrosis

A number of commercially-available mice strains can be used in various models of cystic fibrosis. As an example, homozygous B6.129S4-1^{miPds}/J mice are useful in studies of pulmonary infection, pulmonary injury and aneurysm, as well as P. aeruginosa resistance commonly observed in cystic fibrosis patients. This mice strain, as well as a number of other JAX® mice strains, can be obtained readily from Jackson laboratories. Detailed description and protocols for carrying out in vivo evaluation in various murine models of cystic fibrosis can be found in Scholte et al “Animal Models of Cystic Fibrosis” J. Cystic Fibrosis 2004, Aug. 3, Suppl. 2: p. 183-190.

### Example 6

### Effects of Compounds of the Invention in a Mouse Model for Lung Eosinophilia

Male Balb/C mice with an approximate weight of 20 g can be used for the study (n=8). Once randomized, animals are sensitized by an i.p. injection of ovalbumin (OVA, Sigma) on day zero and then subsequently on day 14. On the twenthieth day, mice are subjected to a challenge test by intranasal application of OVA (positive control) or PBS (negative control). 48 hours after the intranasal application of OVA, mice are euthanized. Lungs are removed and rinsed with 1 mL of PBS. The cells can be separated by centrifuge and stained in Diff-Quick (Dade) and the percentage of eosinophils can be determined by differential counting of at least 100 cells. Fluticasone and beclomethasone are used as standard substances, with positive and negative controls. The compounds of the invention can be administered by intranasal or i.p. 2 days before the challenge test and up to the completion of the study.

### Example 7

### In vivo Effects of the Compounds of the Invention in Animal Models to treat Inflammatory Bowel Diseases (IBD) and Crohn's Disease

A number of established mouse and rat models to treat IBD and Crohn's Disease are available. The compounds of the invention can be evaluated in the trinitrobenzene sulfonic acid (TNBS)-induced inflammatory bowel disease in rats or mice. Detailed protocols can be found in Kankuri et al Inflammation 2001, 25, p. 301-310 and Fiorucci et al, Proc. Natl. Acad. Sci. USA 2002, 99, p. 15770-75. Alternatively, the compounds of the invention can be evaluated in the acetic acid-induced acute chemical colitis in rats (see Kim et al, Arch. Pharm. Res. 1999, 22, p. 354-60), in the dextran sulfate (DSS) induced colitis in mice (see van Meeteren et al, Scand. J. Gastroenterol. 2000, 35, p. 517-21), in the SAMP1/Yit mice that spontaneously develop chronic terminal ileitis similar to Crohn's disease (see Matsumoto et al. Gut, 1998, 43, p. 71-78), and in IL-10 deficient mice that develop colitis (see Farmer et al, Proc. Natl. Acad. Sci. USA 2001, 98, p. 13820-25).

### Compounds

The following non-limiting compound examples serve to illustrate further embodiments of the fatty acid macrolide derivatives. it is to be understood that any embodiments listed in the Examples section are embodiments of the fatty acid macrolide derivatives and, as such, are suitable for use in the methods and compositions described above.

### Example 8

### Preparation of (2R,3S,4R,5R,10R,11R,12S,13S,14R)-11-((2S,3R,4S,6R)-4- (dimethylamino)-3-hydroxy-6-methyltetrahydro-2H-pyran-2-yloxy)-6- ((4Z,7Z,10Z,13Z,16Z,19Z)-docosa-4,7,10,13,16,19-hexaenoxyl)-2-ethyl-3,4,10-trihydroxy- 13-((2R,4R,5S,6)-5-hydroxy-4-methoxy-4,6-dimethyltetrahydro-2H-pyran-2-yloxy)- 3,5,8,10,12,14-hexamethyl-1-oxa-6-azacyclopentadecan-15-one (Ia-1)

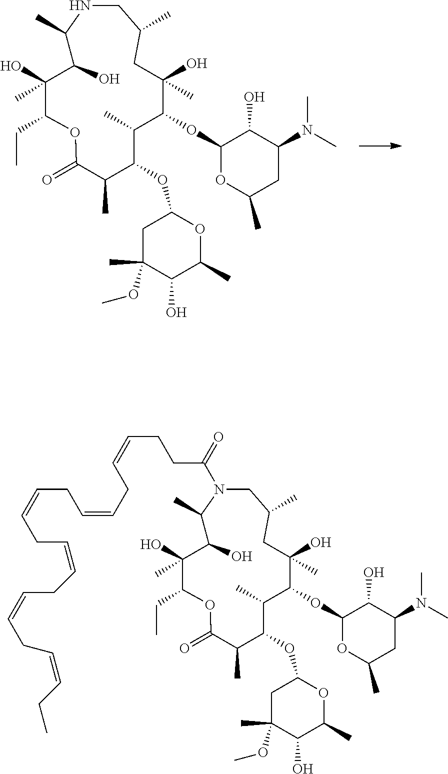 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(O)C[C@@H](C)CN[C@H](C)[C@@H](O)[C@]1(C)O ; CC/C=C\C/C=C\C/C=C\C/C=C\C/C=C\C/C=C\CCC(=O)N1C[C@H](C)C[C@@](C)(O)[C@H](O[C@@H]2O[C@H](C)C[C@H](N(C)C)[C@H]2O)[C@@H](C)[C@H](O[C@H]2C[C@@](C)(OC)[C@@H](O)[C@H](C)O2)[C@@H](C)C(=O)O[C@H](CC)[C@@](C)(O)[C@H](O)[C@H]1C]

9a-N-Desmethyl azithromycin is a well-known precursor to azithromycin and can be obtained from various commercial sources or prepared according to the standard procedures outlined in U.S. Pat. No. 4,517,357 and International Application No. PCT/US2001/000364. 9a-N-Desmethyl azithromycin (100 mg, 0.136 mmol) was taken up in of DMF ( 5 mL) along with (4Z,7Z,10Z,13Z,16Z,19Z)-docosa-4,7,10,16,19-hexaenoic acid (45 mg, 0.136 mmol), HATU (57 mg, 0.41 mmol) and DIEA (36 μL, 0.2 mmol). The resulting reaction mixture was stirred at room temperature for 18 h and diluted with EtOAc. The organic layer was washed with saturated aqueous NaHCO₃, brine, dried over Na₂SO₄, filtered and concentrated under reduced pressure. Purification by silica gel chromatography (5% MeOH/CH₂Cl₂) afforded 40 mg of (2R,3S,4R,8R,10R,11R,12S,13S,14R)-11- ((2S,3R,4S,6R)-4-(dimethylamino)-3-hydroxy-6-methyltetrahydro-2H-pyran-2-yloxy)-6- ((4Z,7Z,10Z,13Z,16Z,19)-docosa-4,7,10,13,16,19-hexaenoyl)-2-ethyl-3,4,10-trihydroxy-13- ((2R,4R,5S,6)-5-hydroxy-4-methoxy-4,6-dimethyltetrahydro-2H-pyran-2-yloxy)- 3,5,8,10,12,14-hexamethyl-1-oxa-6-azacyclopentadecan-15-one. MS calculated for C₅₉H₁₀₀N₂O₁₃: 1044.72; found: 1044.56 [M⁺+1];

¹H NMR (400 MHz, CDCl₃) δ 1.49-0.83 (m, 40H), 2.10-2.00 (m, 3H), 2.41-2.11 (m, 13H), 2.82-2.60 (m, 10H), 2.93-2.98 (m, 1H), 3.35-3.12 (m, 4H), 3.61-3.57 (m, 4H), 4.03-3.97 (m, 2H), 4.33 (m, 1H), 4.74-4.71 (m, 1H), 4.98-4.92 (m, 1), 5.30-5.21 (m, 12H).

### Example 9

### Preparation of (2R,3S,4R,5R,8R,10R,11R,12S,13S,14R)-11-(((2S,3R,4S,6R)-4- (dimethylamino)-3-hydroxy-6-methyltetrahydro-2H-pyran-2-yl)oxy)-6- ((4Z,7Z,10Z,13Z,16Z,19Z)-docosa-4,7,10,13,16,19-hexaenoyl)-2-3,4,10,13- tetrahydroxy-3,5,8,10,12,14-hexamethyl-1-oxa-6-azacyclopentadecan-15-one (Ia-2)

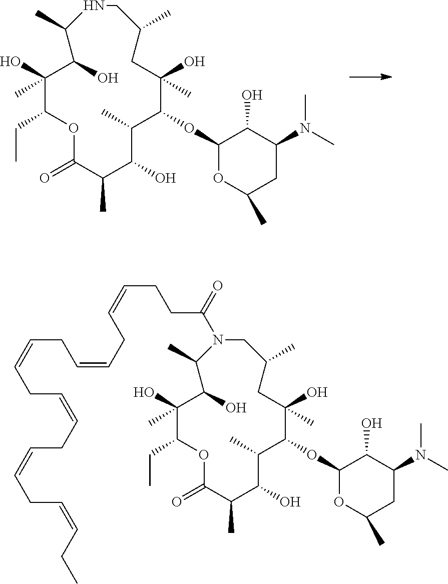 [SMILES: CC[C@H]1OC(=O)[C@H](C)[C@@H](O)[C@H](C)[C@@H](O[C@@H]2O[C@H](C)C[C@H](N(C)C)[C@H]2O)[C@](C)(O)C[C@@H](C)CN[C@H](C)[C@@H](O)[C@]1(C)O ; CC/C=C\C/C=C\C/C=C\C/C=C\C/C=C\C/C=C\CCC(=O)N1C[C@H](C)C[C@@](C)(O)[C@H](O[C@@H]2O[C@H](C)C[C@H](N(C)C)[C@H]2O)[C@@H](C)[C@H](O)[C@@H](C)C(=O)O[C@H](CC)[C@@](C)(O)[C@H](O)[C@H]1C]

(2R,3S,4R,5R,8R,10R,11R,12S,13S,14R)-11-(((2S,3R,4S,6R)-4- (dimethylamino)-3-hydroxy-6-methyltetrahydro-2H-pyran-2-yl)oxy)-2-ethyl-3,4,10,13- tetrahydroxy-3,5,8,10,12,14-hexamethyl-1-oxa-6-azacyclopentadecan-15-one was obtained from 9a-N-desmethyl azithromycin as follows:

9a-N-Desmethyl azithromycin (20 g, 27.2 mmol) was dissolved in 1.0 L of MeOH, and then 27.2 mL of conc. HCl was added dropwise. The reaction mixture was stirred at room temperature for 2 days. LC/MS showed that the reaction was completed. After neutralization with sodium hydrogen carbonate, the resulting mixture was concentrated under reduce pressure to remove any volatile solvents. The residue was diluted with CH₂CL₂ (500 mL) and extracted with dilute 2M aq. HCl (3×200 mL). The pH of the combined aqueous layers was brought up to 10 with 20% aqueous NaOH and the resulting mixture was extracted with CH₂Cl₂ (3×mL). The combined organic layers was dried over anhydrous Na₂SO₄ and concentrated under reduced pressure to afford 13.5 g of (2R,3S4R,5R,8R,10R,11R,12S,13S,14R)-11-(((2S,3R,4S,6R)-4-(dimethylamino)-3- hydroxy-6-methyltetrahydro-2H-pyran-2-yl)oxy)-2-ethyl-3,4,10,13-tetrahydroxy- 3,5,8,10,12,14-hexamethyl-1-oxa-6-azacyclopentadecan-15-one.

MS calculated for C₂₉H₅₆N₂O₉; 576.30; found: 577.3 [M⁺+1]; ¹H NMR (400 MHz, CDCl₃) δ 0.83-1.38 (m, 36H), 1.55-1.96 (m, 9H), 2.24-2.26 (m, 10H), 2.45-2.80 (m, 6H), 3.07 (s, 1H), 3.25-3.61 (m, 6H), 3.79-4.02 (m, 4H), 4.45-4.47 (m, 1H), 4.77-4.80 (m, 1H).

(2R,3S,4R,8R,10R,11R,12S,13S,14R)-11-(((2S,3R,4S,6R)-4- (dimethylamino)-3-hydroxy-6-methyltetrahydro-2H-pyran-2-yl)oxy)-2-ethyl-3,4,10,13- tetrahydroxy-3,5,8,10,12,14-hexamethyl-1-oxa-6-azacyclopentadecan-15-one (1.16 g, 2.0 mmol) was taken up in 10 mL of CH₂Cl₂ and 4 mL of DMF along with DHA (650 mg, 2.0 mmol), HATU (760 mg, 2.0 mmol) and DIEA (0.4 mL, 4.2 mmol). The resulting reaction mixture was stirred at room temperature for 18 h and then diluted with CH₂Cl₂ (50 mL). The organic layer was washed with brine, dried over anhydrous Na₂SO₄ and concentrated under reduced pressure. The resulting residue was purified by preparative-HPLC to afford 110 mg of (2R,3S,4R, 5R,8R, 10R,11R,12S,13S,14R)-11-(((2S,3R,4S,6R)-4-dimethylamino)-3- hydroxy-6-methyltetrahydro-2H-pyran-2-yl)oxy)-6-((4Z,7Z,10Z,13Z,16Z,19Z)-docosa- 4,7,10,13,16,19-hexaenoyl)-2-ethyl-3,4,10,13-tetrahydroxy-3,5,8,10,12,14-hexamethyl-1- oxa-6-azacyclopentadecan-15-one (Yield; 6.2%).

MS calculated for C₅₁H₈₆N₂O₁₀: 886.62; found: 887.3 [M⁺+1]; ¹H NMR (400 MHz, CDCl₃) δ 5.42-5.33 (m, 12H), 4.84-4.55 (m, 2H), 3.74-3.59 (m, 2H), 2.84-2.80 (m, 10H), 2.76-2.20 (m, 10H), 2.15-2.02 (m, 2H), 1.98 -1.45 (m, 4H), 1.40-1.21 (m, 9H), 1.10-0.81 (m, 9H).

### Example 10

### Preparation of (2R,3S,4R,5R,8R,10R,11R,12S,13S,14R)-6-((4Z,7Z,10Z,13Z,16Z,19Z)- docosa-4,7,10,13,16,19-hexaenoyl)-2-ethyl-3,4,10,11,13-pentahydroxy-3,5,8,10,12,14- hexamethyl-1-oxa-6-azacyclopentadecan-15-one (Ia-3)

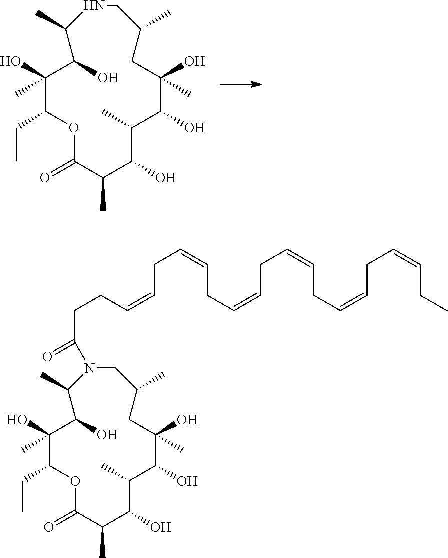 [SMILES: CC[C@H]1OC(=O)[C@H](C)[C@@H](O)[C@H](C)[C@@H](O)[C@](C)(O)C[C@@H](C)CN[C@H](C)[C@@H](O)[C@]1(C)O ; CC/C=C\C/C=C\C/C=C\C/C=C\C/C=C\C/C=C\CCC(=O)N1C[C@H](C)C[C@@](C)(O)[C@H](O)[C@@H](C)[C@H](O)[C@@H](C)C(=O)O[C@H](CC)[C@@](C)(O)[C@H](O)[C@H]1C]

(2R,3S,4R,8R,10,11R,12S,13S,14R)-2-Ethyl-3,4,10,11,13-pentahydroxy- 3,5,8,10,12,14-hexamethyl-1-oxa-6-azacyclopentadecan-15-one was prepared as follows: (2R,3S,4R,5R,8R,10R,11R,12S,13S,14R)-11-(((2S,3R,4S,6R)-4-(dimethylamino)-3- hydroxy-6-methyltetrahydro-2H-pyran-2-yl)oxy)-2-ethyl-3,4,10,13-tetrahydroxy- 3,5,8,10,12,14-hexamethyl-1-oxa-6-azacyclopentadecan-15-one (10.0 g, 17.3 mmol) was taken up in CH₂Cl₂ (75 mL) and 150 mL of 6 M HCl and added. The resulting reaction mixture was stirred under reflux for 18 h. Once the reaction mixture had cooled to room temperature, the pH was adjusted to 5 with 20% az. NaOH. The aqueous layer was separated and washed with CH₂Cl₂. The extractions with CH₂Cl₂ were repeated when the pH was adjusted to 7.0, and then again when the pH was adjusted to 11.0. The combined organic extracts at pH=11 were dried over anhydrous Na₂SO₄ and concentrated under reduced pressure to afford 4.5 g of (2R,3S,4R,5R,8R,10R,11R,12S,13S,14R)-2-ethyl-3,4,10,11,13- pentahydroxy-3,5,8,10,12,14-hexamethyl-1-oxa-6-azacyclopentadecan-15-one as a white solid. Yield: 61.9%

MS calculated for C₂₁H₄₁NO₇: 419.29; found: 420.3 [M⁺+1];

¹H NMR (400 MHz, CDCl₃) δ 0.83-1.38 (m, 20H), 1.45-1.59 (m, 3H), 1.73-1.96 (m, 3H), 2.15-2.28 (m, 2H), 2.51-2.73 (m, 3H), 3.10-3.15 (m, 2H), 3.52-3.56 (m, 2H), 3.76 (d, J=10.4 Hz, 1H), 4.87 (dd, J=2.0, 10.9 Hz, 1H).

To a solution of DHA (660 mg, 2.0 mmol) in 10 mL of CH₂Cl₂ and 4 mL of DMF was added HATU (760 mg, 2.0 mmol) and the mixture was stirred for 10 minutes, (2R,3S,4R,5R,8R,10R,11R,12S,13S,14R)-2-Ethyl-3,4,10,11,13-pentahydroxy- 3,5,8,10,12,14-hexamethyl-1-oxa-6-azacyclopentadecan-15-one (843 mg, 2.0 mmol) and DIEA (0.4 mL, 4.2 mmol) were then added. The resulting reaction mixture was stirred at room temperature for 18 h. Then the reaction mixture was diluted with CH₂Cl₂ and washed with brine. The organic phase was dried over anhydrous. Na₂SO₄ and concentrated under reduced pressure. The resulting residue was purified by preparative-HPLC to afford 278 mg of (2R,3S,4R,5R,8R,10R,11R,12S,13S,14R)-6-((4Z,7Z,10Z,13Z,16Z,19Z)-docosa- 4,7,10,13,16,19-hexaenoyl)-2-ethyl-3,4,10,11,13-pentahydroxy-3,5,8,10,12,14-hexamethyl-1- oxa-6-azacyclopentadecan-15-one (Yield: 19.0%)

MS calculated for C₄₃H₇₁NO₈: 729.52; found: 730.4 [M⁺+1]; ¹H NMR (400 MHz, CHCl₃) δ 5.43-5.30(m, 12H), 4.35-3.95 (m, 2H), 3.64-3.50 (m, 2H), 2.91-2.80 (m, 10H), 2.76-2.61 (m, 2H), 2.42-2.29 (m, 5H), 2.18-1.85 (m, 4H), 1.70-1.51 (m, 7H), 1.40-0.81 (m, 27H).

### Example 11

### Preparation of (4Z,7Z,10Z,13Z,16Z,19Z)-N-(3- ((2R,3S,4R,5R,8R,10R,11R,12S,13S,14R)-11-(((2S,3R,4S,6R)-4-(dimethylamino)-3- hydroxy-6-methyltetrahydro-2H-pyran-2-yl)oxy)-2-ethyl-3,4,10-trihydroxy-13- (((2R,4R,5S,6S)-5-hydroxy-4-methoxy-4,6-dimethyltetrahydro-2H-pyran-2-yl)oxy)- 3,5,8,10,12,14-hexamethyl-15-oxo-1-oxa-6-azacyclopentadecan-6-yl)-3- oxopropyl)docosa-4,7,10,13,16,19-hexaenamide (Ia-5)

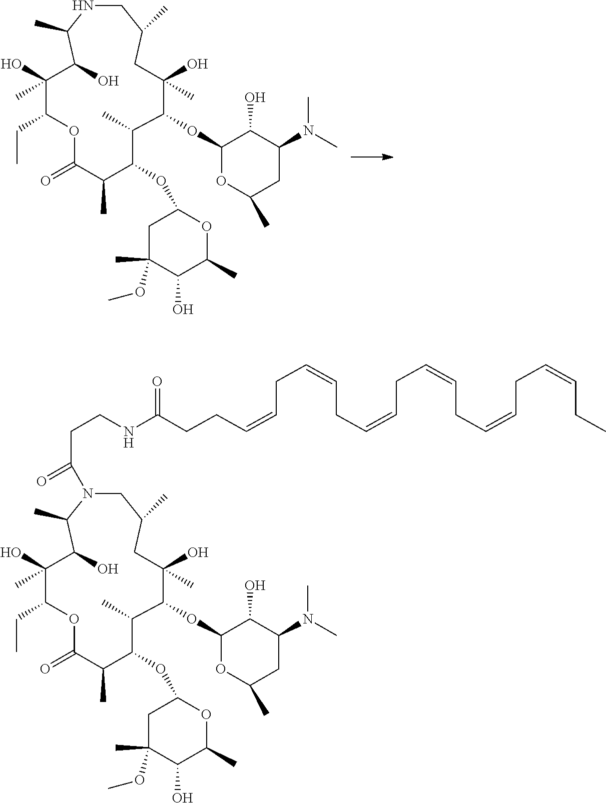 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(O)C[C@@H](C)CN[C@H](C)[C@@H](O)[C@]1(C)O ; CC/C=C\C/C=C\C/C=C\C/C=C\C/C=C\C/C=C\CCC(=O)NCCC(=O)N1C[C@H](C)C[C@@](C)(O)[C@H](O[C@@H]2O[C@H](C)C[C@H](N(C)C)[C@H]2O)[C@@H](C)[C@H](O[C@H]2C[C@@](C)(OC)[C@@H](O)[C@H](C)O2)[C@@H](C)C(=O)O[C@H](CC)[C@@](C)(O)[C@H](O)[C@H]1C]

DHA (3 g, 9.14 mmol) was taken up in 50 mL of CH₂Cl₂ along with HOBt (1.85 g, 13.71 mmol), EDCI (2.62 g, 13.71 mmol), beta-alanine methyl esther hydrochloride (1.40 g, 10.06 mmol) and DIEA (3.53 g, 27.42 mmol). The resulting reaction mixture was stirred at room temperature for 18 h. It was then diluted with CH₂Cl₂ (50 mL) and washed with aqueous (NH₄Cl (3×100 mL) and brine (3×100 mL). The organic layer was dried over anhydrous NaSO₄ and concentrated under reduced pressure. The resulting residue was purified by silica gel chromatography (ethyl acetate, gradient elution to 60% Petroleum ether, 40% ethyl acetate) to afford 3.3 g of methyl 3-(4Z,7Z,10Z,13Z,16Z,19)-docosa- 4,7,10,13,16,19-hexaenamidopropanoate (Yield: 87.5).

MS calculated for C₂₆H₃₉NO₃: 413.59; found: 414.01 [M⁺+1].

Methyl 3-(4Z,7Z,10Z,13Z,16Z,19)-docosa-4,7,10,13,16,19- hexaenamidopropanoate (3.3 g, 7.99 mmol) was taken up in 76 mL of THF along with an aqueous solution of NaOH (1.27 g in 76 mL of H₂O). The resulting reaction mixture was stirred at room temperature for 5 h. It was then acidified to pH 4 with 2 N HCl and then extracted with ethyl acetate and washed with brine (5×100 mL). The organic layer was dried (Na₂SO₄) and concentrated under reduced pressure to afford 3.0 g of 3- (4Z,7Z,10Z,13Z,16Z,19Z)-docosa-4,7,10,13,16,19-hexaenamidopropanoic acid (Yield: 96.7%).

MS calculated for C₂₅H₃₇NO₃: 399.56; found: 400.01 [M⁺+1].

To a solution of 3-(4Z,7Z,10Z,13Z,16Z,19Z)-docosa-4,7,10,13,16,19- hexaenamidopropanoic acid (0.81 g, 2.04 mmol) in 20 mL of Ch₂Cl₂ and 8 mL of DMF was added HATU (0.85 g, 2.24 mmol) and the mixture was stirred for 10 minutes. Then 9a-N- desmethyl azithromycin (1.5 g, 2.04 mmol) and DIEA (0.43 g, 3.36 mmol) were added. The reaction mixture was stirred at room temperature for 18 h and then diluted with CH₂Cl₂. The organic layer was washed with brine, dried over anhydrous Na₂SO₄ and concentrated under reduced pressure. The resulting residue was purified by preparative-HPLC to afford 90 mg of (4Z,7Z,10Z,13Z,16Z,19Z)-N-(3-((2R,3S,4R,5R,8R,10R,11R,12S,13S,14R)-11- (((2S,3R,4S,6R)-4-(dimethylamino)-3-hydroxy-6-methyltetrahydro-2H-pyran-2-yl)oxy)-2- ethyl-3,4,10-trihydroxy-13-(((2R,4R,5S,6S)-5-hydroxy-4-methoxy-4,6-dimethyltetrahydro- 2H-pyran-2-yl)oxy)-3,5,8,10,12,14-hexamethyl-15-oxo-1-oxa-6-azacyclopentadecan-6-yl)-3- oxopropyl)docosa-4,7,10,13,16,19-hexaenamide (Yield: 3.9%).

MS calculated for C₆₂H₁₀₅N₃O₁₄: 1116.5; found: 1116.7 [M⁺+1]; ¹H NMR (400 MHz, CHCl₃) δ 1.75 (m, 36H), 1.97-1.86 (m, 8H), 2.14-2.06 (m, 3H), 2.34-2.29 (m, 15H), 2.41 (s, 3H), 2.94-2.59 (m, 16H), 3.42-3.33 (m, 5H), 3.54-3.44 (m, 5H), 4.22-3.99 (s, 1H), 4.32-4.28 (s, 1H), 4.93 (s, 1H), 5.21-4.94 (s, 1H), 5.32-5.22 (m, 12H).

### Example 12

### Preparation of (5Z,8Z,11Z,14Z,17Z)-N-((S)-1-((2R,3S,4R,5R,8R,10R,11R,12S,13S,14R)- 11-(((2S,3R,4S,6R)-4-(dimethylamino)-3-hydroxy-6-methyltetrahydro-2H-pyran-2- yl)oxy)-2-ethyl-3,4,10,13-tetrahydroxy-3,5,8,10,12,14-hexamethyl-15-oxo-1-oxa-6- azacyclopentadecan-6-yl)-1-oxopropan-2-yl)icosa-5,8,11,14,17-pentaenamide (Ia-6)

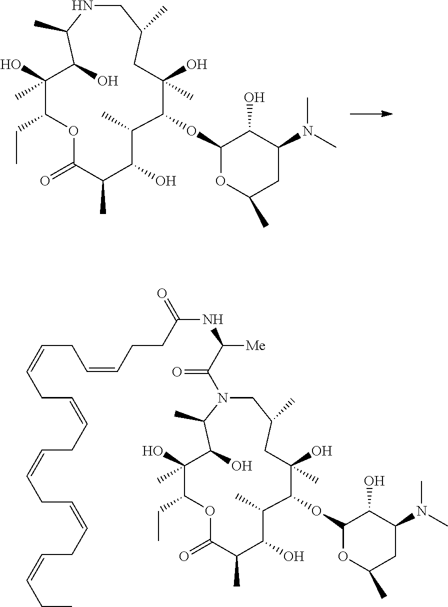

DHA (7.0 g, 21.34 mmol) was taken up in 80 mL of CH₂Cl₂ along with HOBt (4,32 g, 32.01 mmol), EDCI (6.13 g, 32.01 mmol), L-alanine methyl ester hydrochloride (3.27 g, 23.47 mmol) and DIEA (8.25 g, 64.02 mmol). The resulting reaction mixture was stirred at room temperature for 18 h. It was then diluted with CH₂Cl₂ (80 mL) and washed with aq. NH₄Cl (3×100 mL) and brine (3×100 mL). The organic layer was dried over anhydrous Na₂ SO ₄ and concentrated under reduced pressure. The resulting residue was purified by silica gel chromatography (ethyl acetate, gradient elution to 60% Petroleum ether, 40% ethyl acetate) to afford 7.35 g of (S)-methyl 2-((4Z,7Z,10Z,13Z,16Z,19Z)-docosa- 4,7,10,13,16,19-hexaenamido)propanoate (Yield: 83.05%).

MS calculated for C₂₆H₃₉NO₃: 413.59; found: 414.10 [M+H]⁻.

(S)-Methyl 2-((4Z,7Z,10Z,13Z,16Z,19Z)-docosa-4,7,10,13,16,19- hexaenamido)propanoate (7.35 g, 17.79 mmol) was taken up in 170 mL of THF along with an aqueous solution of NaOH (2.48 g in 170 mL of H₂O). The resulting reaction mixture was stirred at room temperature for 5 h. It was then acidified to pH 4 with 2 N HCl and then extracted with ethyl acetate. The combined organic layers were washed with brine (5×250 mL), dried (Na₂SO₄) and concentrated under reduced pressure to afford 6.98 g of (S)-2- ((4Z,7Z,10Z,13Z,16Z,19Z)-docosa-4,7,10,13,16,19-hexaenamido)propanoic acid (Yield: 96.9%).

(2R,3S,4R,5R,8R,10R,11R,12S,13S,14R)-11-(((2S,3R,4S,6R)-4- (dimethylamino)-3-hydroxy-6-methyltetrahydro-2H-pyran-2-yl)oxy)-2-ethyl-3,4,10,13- tetrahydro-3,5,8,10,12,14-hexamethyl-1-oxa-6-azacyclopentadecan-15-one (100 mg, 0.173 mmol) was taken up in 5 mL of CH₃CN along with (S)-2-((4Z,7Z,10Z,13Z,16Z,19Z)-docosa- 4,7,10,13,16,19-hexaenamido)propanoic acid (69 mg, 0.173 mmol), HATU (72 mg, 0.19 mmol) and DIEA (42 μL, 0.52 mmol). The resulting reaction mixture was stirred at room temperature for 18 h. It was then diluted with EtOAc and washed with brine. The organic layer was dried (Na₂SO₄) and concentrated under reduced pressure. Purification by chromatography (95% CH₂Cl₂5% MeOH) afforded 40mg of (5Z,8Z,11Z,14Z,17Z)-N-((S)- 1-((2R,3S,4R,5R,8R,10R,11R,12S,13S,14R)-11-(((2S,3R,4S,6R)-4-dimethylamino)-3- hydroxy-6-methyltetrahydro-2H-pyran-2-yl)oxy)-2-ethyl-3,4,10,13-tetrahydroxy- 3,5,8,10,12,14-hexamethyl-15-oxo-1-oxa-6-azacyclopentadecan-6-yl)-1-oxopropan-2- yl)icosa-5,8,11,14,17-pentaenamide. MS calculated for C₅₄H₉₁N₃O₁₁: 957.67; found: 958 [M⁺+1].

### Example 13

### Preparation of (4Z,7Z,10Z,13Z,16Z,19Z)-N-(3- ((2R,3S,4R,5R,8R,10R,11R,12S,13S,14R)-11-(((2S,3R,4S,6R)-4-(dimethylamino)-3- hydroxy-6-methyltetrahydro-2H-pyran-2-yl)oxy)-2-ethyl-3,4,10-trihydroxy-13- (((2R,4R,5S,6S)-5-hydroxy-4-methoxy-4,6-dimethyltetrahydro-2H-pyran-2-yl)oxy)- 3,5,8,10,12,14-hexamethyl-15-oxo-1-oxa-6-azacycloentadecan-6-yl)propyl)docosa- 4,7,10,13,16,19-hexaenamide (Ia-7)

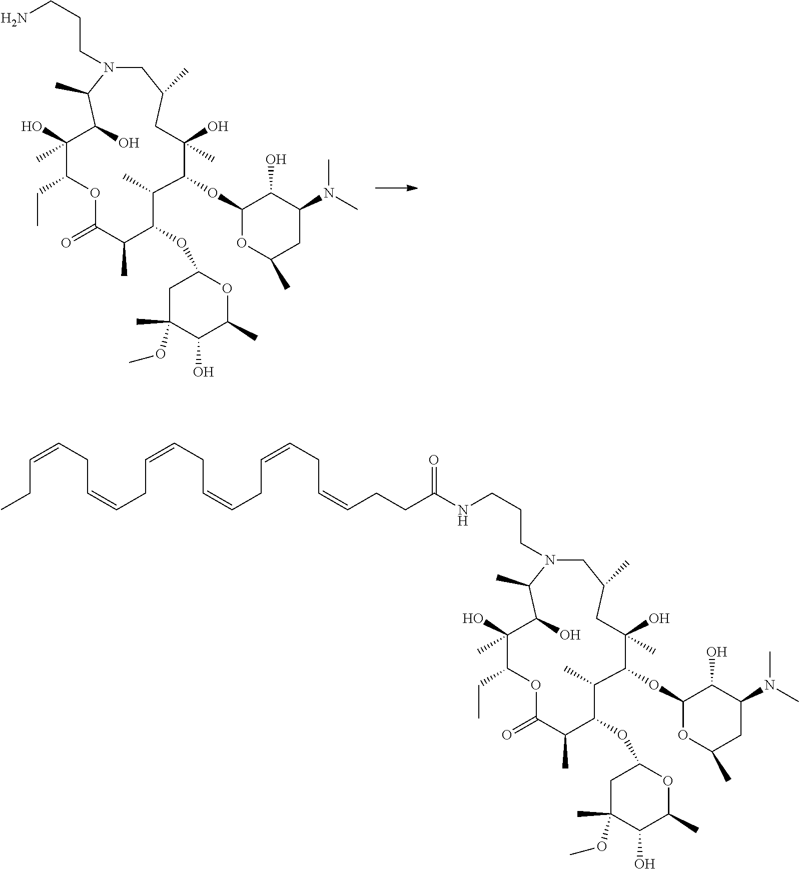 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(O)C[C@@H](C)CN(CCCN)[C@H](C)[C@@H](O)[C@]1(C)O ; CC/C=C\C/C=C\C/C=C\C/C=C\C/C=C\C/C=C\CCC(=O)NCCCN1C[C@H](C)C[C@@](C)(O)[C@H](O[C@@H]2O[C@H](C)C[C@H](N(C)C)[C@H]2O)[C@@H](C)[C@H](O[C@H]2C[C@@](C)(OC)[C@@H](O)[C@H](C)O2)[C@@H](C)C(=O)O[C@H](CC)[C@@](C)(O)[C@H](O)[C@H]1C]

(2R,3S,4R,5R,8R,10R,11R,12S,13S,14R)-6-(3-aminopropyl)-11- (((2S,3R,4S,6R)-4-(dimethylamino)-3-hydroxy-6-methyltetrahydro-2H-pyran-2-yl)oxy)-2- ethyl-3,4,10-trihydroxy-13-(((2R,4R,5S,6S)-5-hydroxy-4-methoxy-4,6-dimethyltetrahydro- 2H-pyran-2-yl)oxy)-3,5,8,10,12,14-hexamethyl-1-oxa-6-azacyclopentadecan-15-one was prepared as follows:

9a-N-Desmethyl azithromycin (10 g, 13.6 mmol) was dissolved in 50 mL of acrylonitrile and the resulting reaction mixture was stirred at 100° C. for 18 h. Upon cooling to room temperature, the reaction mixture was concentrated under reduced pressure to afford 10.5 g of the crude nitrile intermediate. This material was dissolved in 50 mL of AcOH, and 1.0 g of PtO₂ added. The resulting reaction mixture was thoroughly purged with nitrogen and then hydrogenated under 6 atm of hydrogen at room temperature for 24 hours.

The reaction mixture was filtered through a pad of Celite and the filtrate was concentrated under reduced pressure. The resulting residue was purified by column chromatography on silica gel to afford 8.0 g of the amine intermediate, namely (2R,3S,4R,5R,8R,10R,11R,12S,13S,14R)-6-(3-aminopropyl)-11-(((2S,3R,4S,6R)-4- (dimethylamino)-3-hydroxy-6-methyltetrahydro-2H-pyran-2-yl)oxy)-2-ethyl-3,4,10- trihydroxy-13-(((2R,4R,5S,6S)-5-hydroxy-4-methoxy-4,6-dimethyltetrahydro-2H-pyran-2- yl)oxy)-3,5,8,10,12,14-hexamethyl-1-oxa-6-azacyclopentadecan-15-one. Yield: 74.3% MS calculated for C₄₀H₇₇N₃O₁₂: 791.55; found: 792.3 [M⁺+1];

¹H NMR (400 MHz, CDCl₃) δ 0.83-1.38 (m, 40H), 1.41-2.11 (m, 10H), 2.24-2.58 (m, 14H), 2.60-3.11 (m, 7H), 3.21-3.35 (m,5H), 3.55-3.65 (m, 4H), 4.05-4.20 (m, 2H), 4.45-4.47 (m, 1H), 4.90-5.08 (m, 2H);

To a solution of DHA (0.41 g, 1.26 mmol) in 20 mL of CH₂Cl₂ and 8 mL of DMF was added HATU (0.52 g, 1.38 mmol) and the mixture was stirred for 10 minutes, (2R,3S,4R,5R,8R,10R,11R,12S,13S,14R)-6-(3-Aminopropyl)-11-(((2S,3R,4S,6R)-4- (dimethylamino)-3-hydroxy-6-methyltetrahydro-2H-pyran-2-yl)oxy)-2-ethyl-3,4,10- trihydroxy-13-(((2R,4R,5S,6S)-5-hydroxy-4-methoxy-4,6-dimethyltetrahydro-2H-pyran-2- yl)oxy)-3,5,8,10,12,14-hexamethyl-1-oxa-6-azacyclopentadecan-15-one (1.0 g, 1.26 mmol) and DIEA (0.24 g, 1.89 mmol) were then added. The resulting reaction mixture was stirred at room temperature for 18 h and then diluted with Ch₂Cl₂.The organic layer was washed with brine, dried over anhydrous NaSO₄ and concentrated under reduced pressure. The resulting residue was purified by preparative-HPLC to afford 120 mg of (4Z,7Z,10Z,13Z,16Z,19Z)-N- (3-((2R,3S,4R,5R,8R,10R,11R,12S,13S,14R)-11-(((2S,3R,4S,6R)-4-(dimethylamino)-3- hydroxy-6-methyltetrahydro-2H-pyran-2)oxy)-2-ethyl-3,4,10-trihydroxy-13- (((2R,4R,5S,6S)-5-hydroxy-4-methoxy-4,6-dimethyltetrahydro-2H-pyran-2yl)oxy)- 3,5,8,10,12,14-hexamethyl-15-oxo-1-oxa-6-azacyclopentadecan-6-yl)propyl)docosa- 4,7,10,13,16,19-hexaenamide (Yield: 6%).

MS calculated for C₆₂H₁₀₇N₃O₁₃: 1102.52; found: 1102.4 [M⁺+1]; ¹H NMR (400 MHz, CDCl₃) δ 1.49-0.83 (m, 40H), 1.80-1.54 (m, 7H), 2.06-1.87 (m, 9H), 2.41-2.11 (m, 14H), 2.82-2.60 (m, 10H), 2.93-3.17 (m, 3H), 3.25-3.12 (m, 5H), 3.22-3.17 (m, 2H), 3.59-3.57 (d, J=8 Hz, 1H), 3.79-3.66 (s, 1H), 4.09-3.98 (m, 2H), 4.38-4.36 (d, J=7.2 Hz, 1H), 4.59-4.56 (d, J=9.6 Hz, 1H), 4.92-4.91 (d, J=4.4 Hz, 1H), 5.30-5.21 (m, 12H), 5.92 (s, 1H).

### Example 14

### Preparation of (5Z,8Z,11Z,14Z,17Z)-N-(3-((2R,3S,4R,5R,8R,10R,11R,12S,13S,14R)-11- (((2S,3R,4S,6R)-4-dimethylamino)-3-hydroxy-6-methyltetrahydro-2H-pyran-2yl)oxy)- 2-ethyl-3,4,10-trihydroxy-13-(((2R,4R,5S,6S)-5-hydroxy-4-methoxy-4,6- dimethyltetrahydro-2H-pyran-2-yl)oxy)-3,5,8,10,12,14-hexamethyl-15-oxo-1-oxa-6- azacyclopentadecan-6-yl)propyl)icosa-5,8,11,14,17-pentaenamide (Ia-8)

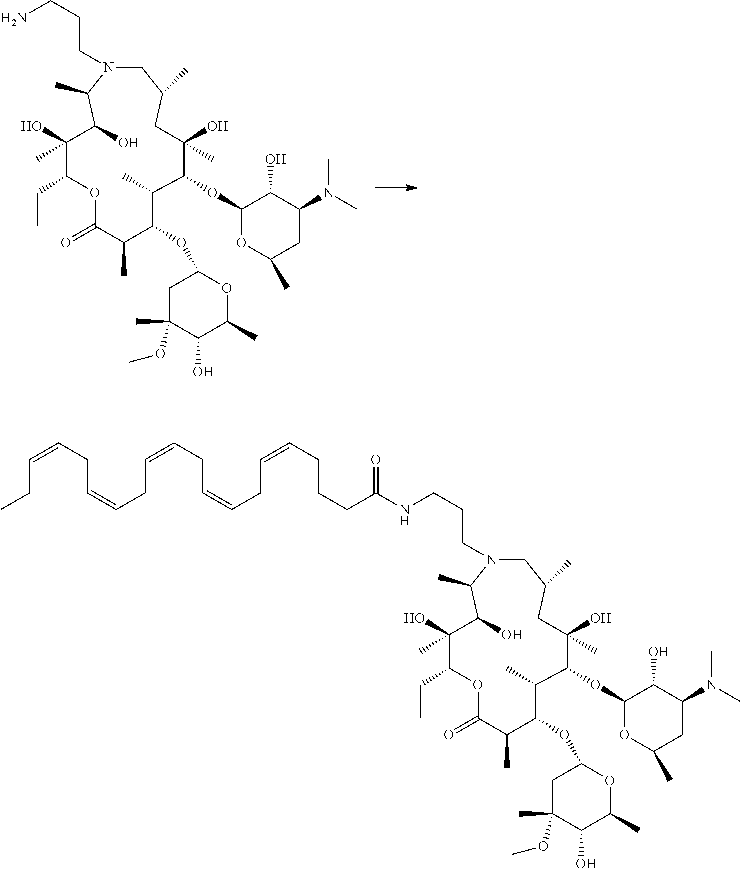 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(O)C[C@@H](C)CN(CCCN)[C@H](C)[C@@H](O)[C@]1(C)O ; CC/C=C\C/C=C\C/C=C\C/C=C\C/C=C\CCCC(=O)NCCCN1C[C@H](C)C[C@@](C)(O)[C@H](O[C@@H]2O[C@H](C)C[C@H](N(C)C)[C@H]2O)[C@@H](C)[C@H](O[C@H]2C[C@@](C)(OC)[C@@H](O)[C@H](C)O2)[C@@H](C)C(=O)O[C@H](CC)[C@@](C)(O)[C@H](O)[C@H]1C]

(5Z,8Z,11Z,14Z,17Z)-N-(3-((2R,3S,4R,5R,8R,10R,11R,12S,13S,14R)-11- (((2S,3R,4S,6R)-4-(Dimethylamino)-3-hydroxy-6-methyltetrahydro-2H-pyran-2-yl)oxy)-2- ethyl-3,4,10-trihydroxy-13-(((2R,4R,5S,6S)-5-hydroxy-4-methyoxy-4,6-dimethyltetrahydro- 2H-pyran-2-yl)oxy)-3,5,8,10,12,14-hexamethyl-15-oxa-1-oxa-6-azacyclopentadecan-6- yl)proply)icosa-3,5,8,10,12,14-pentaenamide was prepared using the same procedure outlined above in example 13, substituting EPA for DHA. MS calculated for C₆₀H₁₀₅N₃O₁₃: 1076.48; found: 1076.4 [M⁺+1];

¹H NMR (400 MHz, CDCl₃) δ 1.35-0.83 (m, 38H), 1.80-1.58 (m, 12H), 2.06-1.87 (m, 15H), 2.41-2.11 (m, 8H), 2.75-2.68 (m, 13H), 2.98-3.20 (m, 3H), 3.25-3.12 (m, 6H), 3.44-3.35 (m, 2H), 3.67-3.57 (m, 3H), 4.10-3.98 (m, 2H), 4.38-4.36 (d, J=7.2 Hz, 1H), 4.58-4.55 (d, J=9.2 Hz, 1H), 4.90-4.89 (d, J=4.8 Hz, 1H), 5.33-5.25 (m, 10H), 5.86 (s, 1H).

### Example 15

### Preparation of (4Z,7Z,10Z,13Z,16Z,19Z)-N-(3- ((2R,3S,4R,5R,8R,10R,11R,12S,13S,14R)-11-(((2R,3R,4S,6R)-4-(dimethylamino)-3- hydroxy-6-methyltetrahydro-2H-pyran-2-yl)oxy)-2-ethyl-3,4,10,13-tetrahydroxy- 3,5,8,10,12,14-hexamethyl-15-oxo-1-oxa-6-azacyclopentadecan-6-yl)propyl)docosa- 4,7,10,13,16,19-hexaenamide (Ia-9)

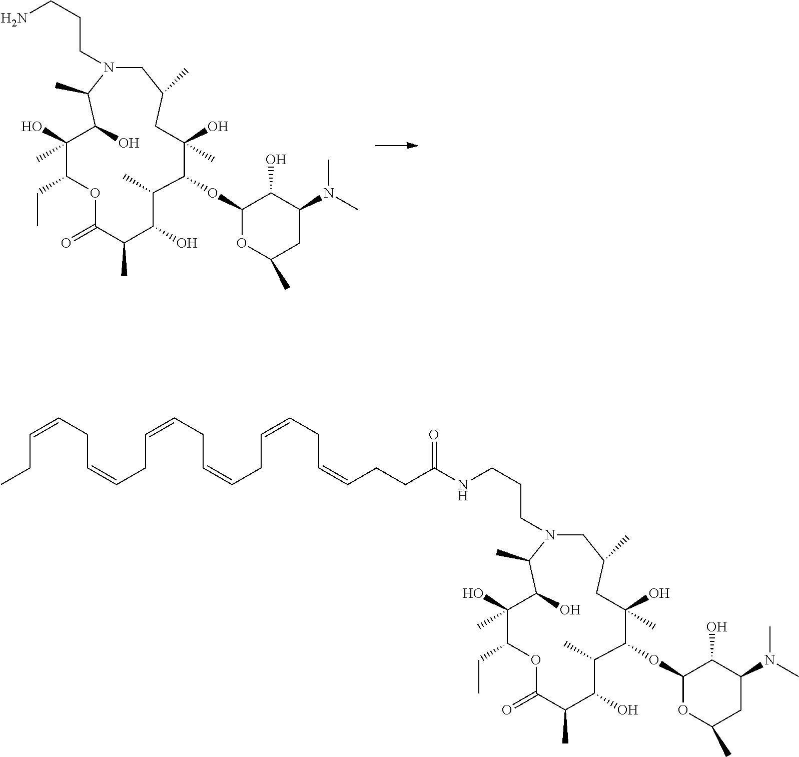 [SMILES: CC[C@H]1OC(=O)[C@H](C)[C@@H](O)[C@H](C)[C@@H](O[C@@H]2O[C@H](C)C[C@H](N(C)C)[C@H]2O)[C@](C)(O)C[C@@H](C)CN(CCCN)[C@H](C)[C@@H](O)[C@]1(C)O ; CC/C=C\C/C=C\C/C=C\C/C=C\C/C=C\C/C=C\CCC(=O)NCCCN1C[C@H](C)C[C@@](C)(O)[C@H](O[C@@H]2O[C@H](C)C[C@H](N(C)C)[C@H]2O)[C@@H](C)[C@H](O)[C@@H](C)C(=O)O[C@H](CC)[C@@](C)(O)[C@H](O)[C@H]1C]

(4Z,7Z,10Z,13Z,16Z,19Z)-N-(3-((2R,3S,4R,5R,8R,10R,11R,12S,13S,14R)-11- (((2S,3R,4S,6R)-4-(Dimethylamino)-3-hydroxy-6-methyltetrahydro-2H-pyran-2yl)oxy)-2- ethyl-3,4,10,13-tetrahydroxy-3,5,8,10,12,14-hexamethyl-15-oxo-1-oxa-6- azacyclopentadecan-6-yl)propyl)docosa-4,7,10,13,16,19-hexaenamide was prepared according to the procedures outlined above in example 13, using the appropriate amine, namely, (2R,3S,4R,5R,8R,10R,11R,12S,13S,14R)-6-(3-aminopropyl)-11-(((2S,3R,4S,6R)-4- (dimethylamino)-3-hydroxy-6-methyltetrahydro-2H-pyran-2-yl)oxy)-2-ethyl-3,4,10,13- tetrahydroxy-3,5,8,10,12,14-hexamethyl-1-oxa-6-azacyclopentadecan-15-one. MS calculated for C₅₄H₉₃N₃O₁₀: 943.69; found: 944.3 [M⁺+1];

¹H NMR (400 MHz, CDCl₃) δ 0.81-1.19 (m, 16H), 1.23-1.35 (m, 9H), 1.40-1.46 (m, 7H), 2.05-2.41 (m, 13H), 2.45-2.75 (m, 14H), 2.80-2.95 (M, 18H), 3.21-3.51 (m, 6H), 3.61-3.81 (m, 4H), 5.26-5.41 (m, 12H).

The amino starting material, namely (2R,3S,4R,8R,10R,11R,12S,13S,14R)-6- (3-aminopropyl)-11-(((2R,3R,4S,6R)-4-(dimethylamino)-3-hydroxy-6-methyltetrahydro-2H- pyran-2-yl)oxy)-2-ethyl-3,4,10,13-tetrahydroxy-3,5,8,10,12,14-hexamethyl-1-oxa-6- azacyclopentadecan-15-one, can be prepared according to the procedures outlined in example 13 using the appropriate macrolide. MS calculated for C₃₂H₆₃N₃O₉: 666.46; found: 643.3 [M⁺+1];

¹H NMR (400 MHz, CD₃OD) δ 0.83-1.59 (m, 24H), 1.85-1.89 (m, 1H), 2.02-2.31 (m, 4H), 2.68-2.90 (m, 10H), 2.99-3.07 (m, 2H), 3.14-3.26 (m, 4H), 3.38-3.55 (m, 1H), 3.60-4.05 (m, 4H), 4.77-5.10 (m, 6H);

### Example 16

### Preparation of (4Z,7Z,10Z,13Z,16Z,19Z)-N-(3- ((2R,3S,4R,5R,8R,10R,11R,12S,13S,14R)-2-ethyl-3,4,10,11,13-pentahydroxy- 3,5,8,10,12,14-hexamethyl-15-oxo-1-oxa-6-azacyclopentadecan-6-yl)propyl)docosa- 4,7,10,13,16,19-hexaenamide (Ia-10)

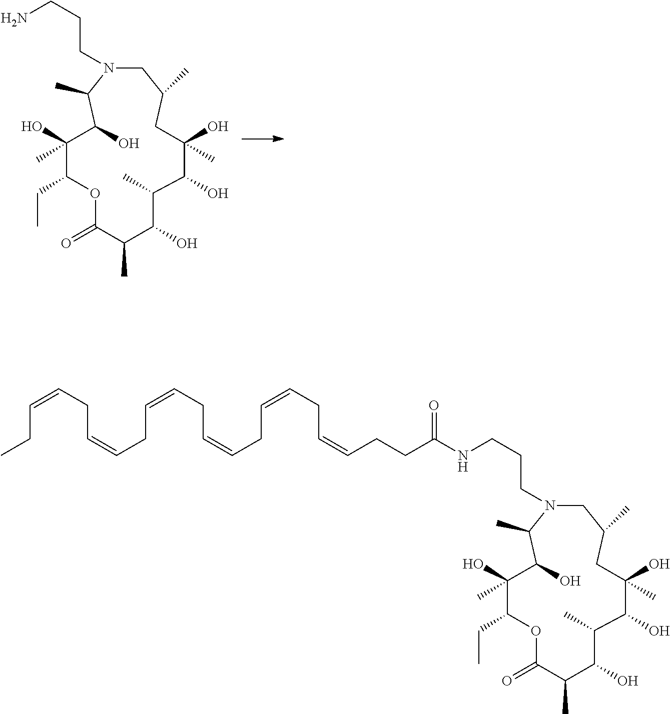 [SMILES: CC[C@H]1OC(=O)[C@H](C)[C@@H](O)[C@H](C)[C@@H](O)[C@](C)(O)C[C@@H](C)CN(CCCN)[C@H](C)[C@@H](O)[C@]1(C)O ; CC/C=C\C/C=C\C/C=C\C/C=C\C/C=C\C/C=C\CCC(=O)NCCCN1C[C@H](C)C[C@@](C)(O)[C@H](O)[C@@H](C)[C@H](O)[C@@H](C)C(=O)O[C@H](CC)[C@@](C)(O)[C@H](O)[C@H]1C]

(4Z,7Z,10Z,13Z,16Z,19Z)-N-(3-((2R,3S,4R,5R,8R,10R,11R,12S,13S,14R)-2- Ethyl-3,4,10,11,13-pentahydroxy-3,5,10,12,14-hexamethyl-15-oxo-1-oxa-6- azacyclopentadecan-6-yl)propyl)docosa-4,7,10,13,16,19-hexaenamide was prepared according to the procedures outlined in example 13 using the appropriate amine starting material, namely (2R,3S,4R,5R,8R,10R,11R,12S,13S,14R)-6-(3-aminopropyl)-2-ethyl- 3,4,10,11,13-pentahydroxy-3,5,8,10,12,14-hexamethyl-1-oxa-6-azacyclopentadecan-15-one. MS calculated for C₄₆H₇₈N₂O₈: 786.57; found: 787.3 [M⁺+1];

¹H NMR (400 MHz, CDCl₃) δ 0.81-1.35 (m, 34H), 1.81-2.41 (m, 24H), 2.81- 2.89 (m, 12H), 3.15-3.25 (m, 4H), 3.61-3.81 (m, 4H), 5.26-5.41 (m, 12H).

This amine starting material, in turn, was prepared according to the procedures outlined in example using the appropriate macrolide. MS calculate for C₂₄H₄₈N₂O₇: 476.3; found: 477.3 [M⁺+1];

¹H NMR (400 MHz, CD₃OD) δ 0.75-1.30 (m, 22H), 1.35-1.58 (m, 4H), 1.65-1.98 (m, 3H), 2.01-2.25 (m, 4H), 2.51-2.81 (m, 6H), 3.31-3.68 (m, 6H), 4.96 (d, J=11.2 Hz, 1H);

### Example 17

### Preparation of (4Z,7Z,10Z,13Z,16Z,19)-N-((2S,3R,4S,6R)-2- (((2R,3S,4R,5R,8R,10R,11R,12S,13S,14R)-2-ethyl-3,4,10-trihydroxy-13-(((2R,4R,5S,6S)- 5-hydroxy-4-methoxy-4,6-dimethyltetrahydro-2H-pyran-2-yl)oxy)-3,5,6,8,10,12,14- heptamethyl-15-oxo-1-oxa-6-azacyclopentadecan-11-yl)oxy)-3-hydroxy-6- methyltetrahydro-2H-pyran-4-yl)-N-methyldocosa-4,7,10,13,16,19-hexaenamide (Ib-1)

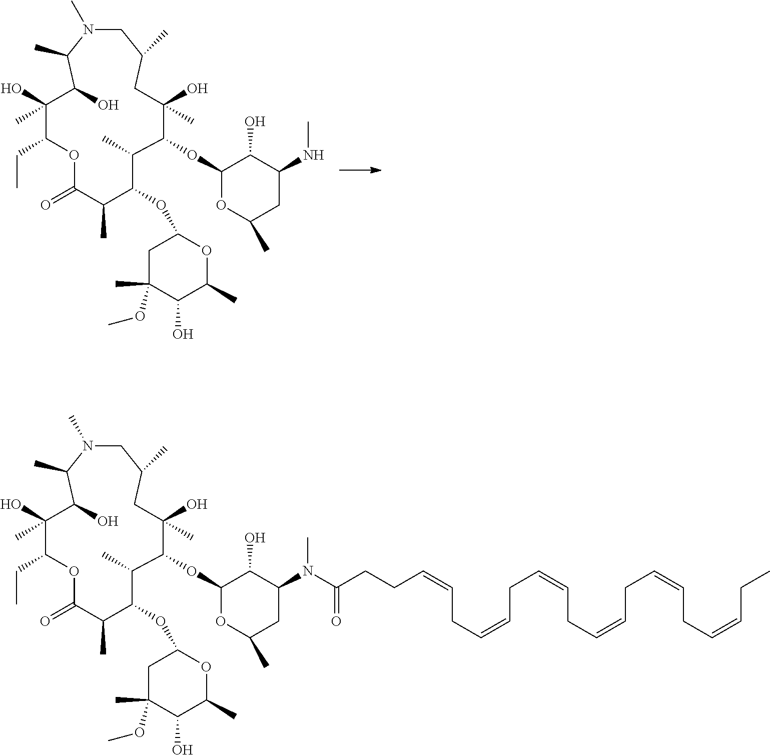

Azithromycin (8.0 g, 10.68 mmol) and sodium acetate (7.42 g, 89.71 mmol) were taken up in 80% aqueous methanol (120 mL). The reaction mixture was heated to 90° C., with stirring, and iodine (2.92 g, 11.53 mmol) was added in three batches within 5 minutes. The mixture was maintained at pH 8-9 by the addition of 1M NaOH (about 8 mL), and poured into ice-cold water containing 5% sodium thiosulfate (120 mL). The resulting mixture was extracted with CH₂Cl₂ (2×50 mL). The aqueous layer was basified with NH₂H₂O, and extracted with 10% CH₃OH in CH₂Cl₂ (3×100 mL). The combined organic layers were dried over anhydrous Na₂SO₄ and concentrated under reduced pressure to afford (2R,3S,4R,5R,8R,10R,11R,12S,13S,14R)-2-ethyl-3,4,10,13-tetrahydroxy-11- (((2R,3R,4S,6R)-3-hydroxy-6-methyl-4-(methylamino)tetrahydro-2H-pyran-2-yl)oxy)- 3,5,6,8,10,12,14-heptamethyl-1-oxa-6-azacyclopentadecan-15-one (7.2 g, Yield: 90%).

MS calculated for C₃₇H₇₀N₃₂O₁₂: 734.95; found: 735.2 [M⁺+1];

¹H NMR (400 MHz, CDCl₃) δ 1.31-0.85 (m, 27H), 2.10-1.7 (m, 10H), 2.47 -2.21 (m, 12H), 2.96-2.94 (m, 3H), 3.25-3.22 (m, 1H), 3.50 -3.47 (m, 1H), 3.59-3.57 (m, 3H), 4.04-3.99 (m, 3H), 4.18-4.17 (m, 1H), 4.34-4.32 (m, 3H), 4.62-4.59 (m, 1H), 4.99-4.98 (m, 16H).

To a solution of DHA (0.45 g, 1.38 mmol) in 20 mL of CH₂Cl₂ and 8 mL of DMF was added HATU (0.58 g, 1.52 mmol) and the mixture was stirred for 10 minutes.

(2R,3S,4R,5R,8R,10R,11R,12S,13S,14R)-2-Ethyl-3,4,10,13-tetrahydroxy-11- (((2S,3R,4S,6R)-3-hydroxy-6-methyl-4-(methylamino)tetrahydro-2H-pyran-2-yl)oxy)- 3,5,6,8,10,14-heptamethyl-1-oxa-6-azacyclopentadecan-15-one (1.2 g, 1.38 mmol) and DIEA (0.27 g, 2.08 mmol) were then added. The resuling reaction mixture was stirred at room temperature for 18 h and then diluted with CH₂Cl₂. The organic layer was washed with brime, dried over anhydrous Na₂SO₄ and concentrated under reduced pressure. The resulting residue was purified by preparative-HPLC to afford 360 mg of (4Z,7Z,10Z,13Z,16Z,19Z)-N- ((2S,3R,4S,6R)-2-(((2R,3S,4R,5R,8R,10R,11R,12S,13S,14R)-ethyl-3,4,10-trihydroxy-13- (((2R,4R,5S,6S)-5-hydroxy-4-methoxy-4,6-dimethyltetrahydro-2H-pyran-2-yl)oxy)- 3,5,6,8,10,12,14-heptamethyl-15-oxo-1-oxa-6-azacyclopentadecan-11-yl)oxy)-3-hydroxy-6- methyltetrahydro-2H-pyran-4-yl)-N-methyldocosa-4,7,10,13,16,19-hexaenamide (Yield: 22.5%).

MS calculated for C₅₉H₁₀₀N₂O₁₃: 1044.72; found: 1045.4 [M⁺+1]; ¹H NMR (400 MHz, CDCl₃) δ 1.42-0.80 (m, 38H), 1.82 -1.49 (m, 6H), 2.00-1.92 (m, 5H), 2.53-2.26 (m, 14H), 2.83-2.62 (m, 15H), 2.97 (t, J=12 Hz, 1H), 3.24 (s, 1H), 3.32 (s,3H), 3.38(s, 1H), 3.60-3.55 (m, 3H), 4.03-3.98 (m, 1H), 4.15-4.14 (m, 1H), 4.47-4.39 (m, 1H), 4.68 -4.58 (m, 1H), 4.96-4.95 (m, 1H), 5.38-5.23 (m, 12H).

### Example 18

### Preparation of (5Z,8Z,11Z,14Z,17Z)-N-((2S,3R,4S,6R)-2- (((2R,3S,4R,5R,8R,10R,11R,12S,13S,14R)-2-ethyl-3,4,10-trihydroxy-13-(((2R,4R,5S,6S)- 5-hydroxy-4-methoxy-4,6-dimethyltetrahydro-2H-pyran-2-yl)oxy)-3,5,6,8,10,12,14- heptamethyl-15-oxo-1-oxa-6-azacyclopentadecan-11-yl)oxy)-3-hydroxy-6- methyltetrahydro-2H-pyran-4-yl)-N-methylicosa-5,8,11,14,17-pentaenamide (Ib-2)

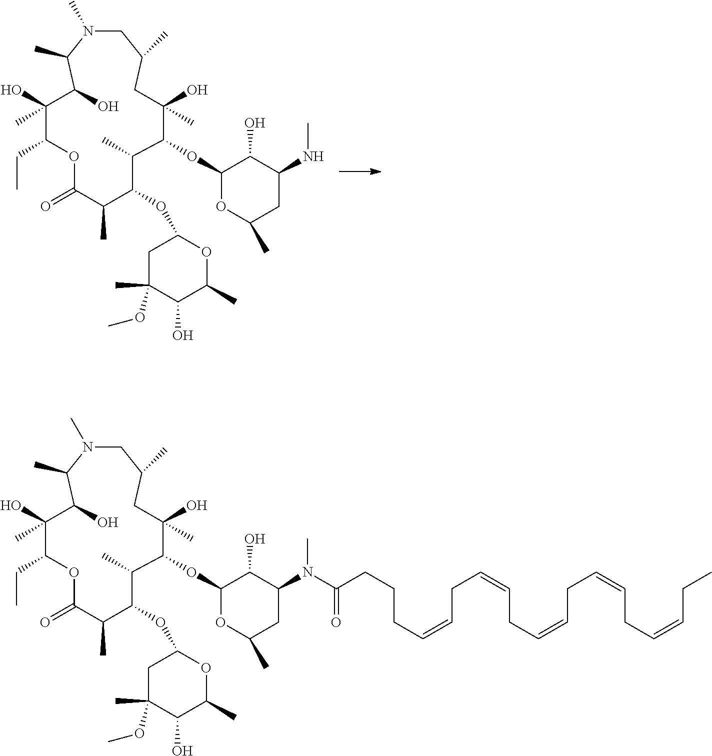

(5Z,8Z,11Z,14Z,17Z)-N-((2S,3R,4S,6R)-2- (((2R,3S,4R,5R,8R,10R,11R,12S,13S,14R)-2-ethyl-3,4,10-trihydroxy-13-(((2R,4R,5S,6S)-5- hydroxy-4-methoxy-4,6-dimethyltetrahydro-2H-pyran-2-yl)oxy)-3,5,6,8,10,12,14- heptamethyl-15-oxo-1-oxa-6-azacyclopentadecan-11-yl)oxy)-3-hydroxy-6-methyltetrahydro- 2H-pyran-4-yl)-N-methylicosa-5,8,11,14,17-pentaenamide was prepared according to the procedures outlined in example 17, using the appropriate EPA starting material. MS calculated for C₅₇H₉₈N₂O₁₃: 1018.71; found: 1019.3 [m⁺+1];

¹H NMR (400 MHz, CDCl₃) δ 1.26-0.86 (m, 43H), 1.70-1.40 (m, 4H), 2.08-1.92 (m, 7H), 2.29-2.23 (m, 6H), 2.45-2.40 (m, 2H), 2.64-2.58 (m, 2H), 2.82-2.74 (m, 13H), 2.97 (t, J=9.6 Hz, 1H), 3.29-3.24 (m, 4H), 3.61-3.57 (m, 3H), 3.79-3.78 (m, 1H), 4.02-4.00 (m, 1H), 4.14-4.13 (m, 1H), 4.46-4.44 (m, 1H), 4.59-4.56 (m, 2H), 4.92-4.91 (m, 1H), 5.34-5.23 (m, 10H).

### Example 19

### Preparation of (4Z,7Z,10Z,13Z,16Z,19Z)-N-((S)-1-(((2S,3R,4S,6R)-2- ((2R,3S,4R,5R,8R,10R,11R,12S,13S,14R)-2-ethyl-3,4,10-trihydroxy-13-(((2R,4R,5S,6S)- 5-hydroxy-4-methoxy-4,6-dimethyltetrahydro-2H-pyran-2-yl)oxy)-3,5,6,8,10,12,14- heptamethyl-15-oxo-1-oxa-6-azacyclopentadecan-11-yl)oxy)-3-hydroxy-6- methyltetrahydro-2H-pyran-4-yl)(methyl)amino)-1-oxopropan-2-yl)docosa- 4,7,10,13,16,19-hexaenamide (Ib-3)

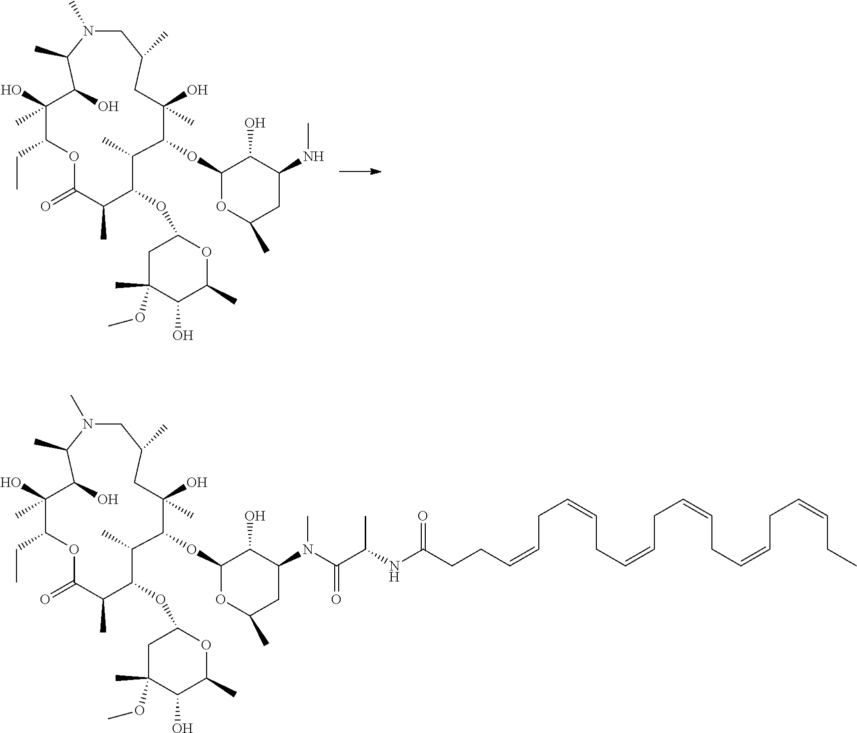

(4Z,7Z,10Z,13Z,16Z,19Z)-N-((S)-1-(((2S,3R,4S,6R)-2- (((2R,3S,4R,5R,8R,10R,11R,12S,13S,14R)-2-ethyl-3,4,10-trihydroxy-13-(((2R,4R,5S,6S)-5- hydroxy-4-methoxy-4,6-dimethyltetrahydro-2H-pyran-2-yl)oxy)-3,5,6,8,10,12,14- heptamethyl-15-oxo-1-oxa-6-azacyclopentadecan-11-yl)oxy)-3-hydroxy-6-methyltetrahydro- 2H-pyran-4-yl)(methyl)amino)-1-oxopropan-2-yl)docosa-4,7,10,13,16,19-hexaenamide was prepared according to the procedure outlined in example 17 using the appropriate acid component, name (S)-2-((4Z,7Z,10Z,13Z,16Z,19Z)-docosa-4,7,10,13,16,19- hexaenamido)propanoic acid. MS calculated for C₆₂H₁₀₅N₃O₁₄: 1116.50; found: 1116.3 [M⁺+1];

¹H NMR (400 MHz, CDCl₃) δ 1.34-0.85 (m, 39H), 1.67-1.47 (m, 6H), 2.09-1.97 (m, 7H), 2.52-2.21 (m, 7H), 2.87-2.65 (m, 14H), 3.08-2.97 (m, 3H), 3.42-3.31 (m, 4H), 3.76-3.64 (m, 4H), 4.08-4.04 (m, 1H), 4.19-4.18 (d, J=6.8 Hz, 1H), 4.65-4.49 (m, 2H), 4.96-4.89 (m, 2H), 5.38-5.30 (m, 12H), 6.57 -6.55 (d, J=7.2 Hz, 1H).

### Example 20

Preparation of (4Z,7Z,10Z,13Z,16Z,19Z)-N-((2S,3R,4S,6R)-2- (((2R,3S,4R,8R,10R,11R,12S,13S,14R)-2-ethyl-3,4,10,13-tetrahydroxy- 3,5,6,8,10,12,14-heptamethyl-15-oxo-1-oxa-6-azacyclopentadecan-11-yl)oxy)-3-hydroxy- 6-methyltetrahydro-2H-pyran-4-yl)-N-methyldocosa-4,5,10,13,16,19-hexaenamide (Ib-4)

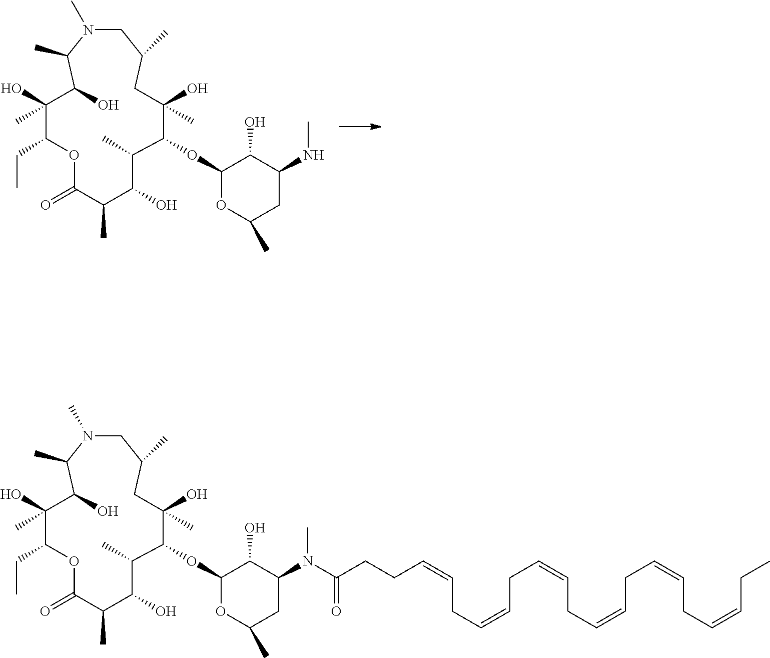

(2R,3S,4R,5R,8R,10R,11R,12S,13S,14R)-2-ethyl-3,4,10,13-tetrahydroxy-11- (((2S,3R,4S,6R)-3-hydroxy-6-methyl-4-(methylamino)tetrahydro-2H-pyran-2-yl)oxy)- 3,5,6,8,10,12,14-heptamethyl-1-oxa-6-azacyclopentadecan-15-one and DHA were subjected to the same reaction conditions outline in example 19 to prepare (4Z,7Z,10Z,13Z,16Z,19Z)- N-(92S,3R,4S,6R)-(((2R,3S,4R,5R,8R,10R,11R,12S,14R)-2-ethyl-3,4,10,13- tetrahydroxy-3,5,6,8,10,12,14-heptamethyl-15-oxo-1-oxa-6-azacyclopentadecan-11-yl)oxy)- 3-hydroxy-6-methyltetrahydro-2H-pyran-4-yl)-N-methyldocosa-4,7,10,13,16,19- hexaenamide. MS calculated for C₅₃H₈₆N₂O₁₀: 886.63; found: 887.5 [M⁺+1];

¹H NMR (400 MHz, CDCl₃) δ 1.31-0.86 (m, 30H), 1.63-1.51 (m,5H), 1.91-1.88 (m, 3H), 2.09-2.02 (m, 3H), 2.43-2.28 (m, 9H), 2.89-2.63 (m, 15H), 3.64-3.60 (d, J=16 Hz, 4H), 3.79-3.76 (d, J=11.2 Hz, 1H), 4.55-4.54 (m, 1H), 4.70-4.67 (m, 1H), 5.39-5.23 (m, 12H).

### Example 21

### Preparation of (5Z,8Z,11Z,14Z,17Z)-N-((2S,3R,4S,6R)-2- (((2R,3S,4R,5R,8R,10R,11R,12S,13S,14R)-2-ethyl-3,4,10.13-tetrahydroxy- 3,5,6,8,10,12,14-heptamethyl-15-oxo-1-oxa-6-azacyclopentadecan-11-yl)oxy)-3-hydroxy- 6-methyltetrahydro-2H-pyran-4-yl)-N-methylicosa-5,8,11,14,17-pentaenamide (Ib-5)

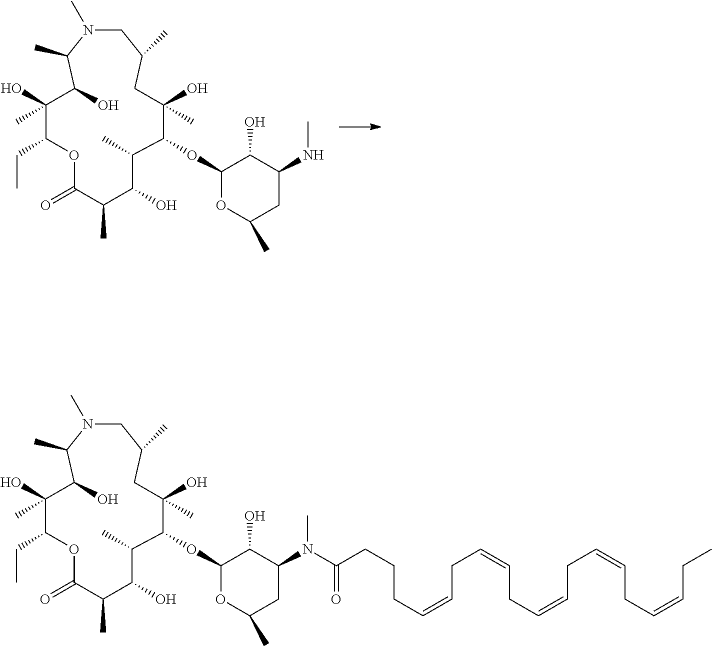 [SMILES: CC[C@H]1OC(=O)[C@H](C)[C@@H](O)[C@H](C)[C@@H](O[C@@H]2O[C@H](C)C[C@H](NC)[C@H]2O)[C@](C)(O)C[C@@H](C)CN(C)[C@H](C)[C@@H](O)[C@]1(C)O ; CC/C=C\C/C=C\C/C=C\C/C=C\C/C=C\CCCC(=O)N(C)[C@H]1C[C@@H](C)O[C@@H](O[C@@H]2[C@@H](C)[C@H](O)[C@@H](C)C(=O)O[C@H](CC)[C@@](C)(O)[C@H](O)[C@@H](C)N(C)C[C@H](C)C[C@@]2(C)O)[C@@H]1O]

(2R,3S,4R,5R,8R,10R,11R,12S,13S,14R)-2-ethyl-3,4,10,13-tetrahydroxy-11- (((2S,3R,4R,5R,6R)-3-hydroxy-4-(methylamino)tetrahydro-2H-pyran-2-yl)oxy)- 3,4,6,8,10,12,14-heptamethyl-1-oxa-6-azacyclopentadecan-15-one and EPA were subjected to the same reaction conditions outline in example 19 to prepare ((5Z,8Z,11Z,14Z,17Z)-N- ((2S,3R,4S,6R)-2-(((2R,3S,4R,5R,8R,10R,11R,12S,13S,14R)-2-ethyl-3,4,10,13- tetrahydroxy-3,5,6,8,10,12,14-heptamethyl-15-oxo-1-oxa-6-azacyclopentadecan-11-yl)oxy)- 3-hydroxy-6-methyltetrahydro-2H-pyran-4-yl)-N-methylicosa-5,8,11,14,17-pentaenamide. MS calculated for C₄₉H₈₄N₂O₁₀: 860.61; found: 861 [M⁺+1];

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A molecular conjugate comprising a macrolide and a fatty acid selected from omega-3 fatty acids, fatty acids metabolized in vivo into omega-3 fatty acids, and lipoic acid.

2. A compound of Formula I: 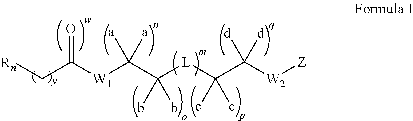
and pharmaceutically acceptable salts, hydrates, solvates, prodrugs, enantiomers, and stereoisomers thereof;
wherein
Rₐ is a macrolide;
W₁ and W₂ are each independently null, O, S, NH, NR, or W₁ and W₂ can be taken together can form an imidazolidine or piperazine group;
each a, b, c, and d is independently —H, —D, —CH₃, —OCH₃, —OCH₂CH₃, —C(O)OR, —O-Z, or benzyl, or two of a, b, c, and d can be taken together, along with the single carbon to which they are bound, to form a cycloalkyl or heterocycle;
w is 0 or 1;
y is 0, 1, 2, or 3;
each n, o, p, and q is independently 0, 1 or 2; 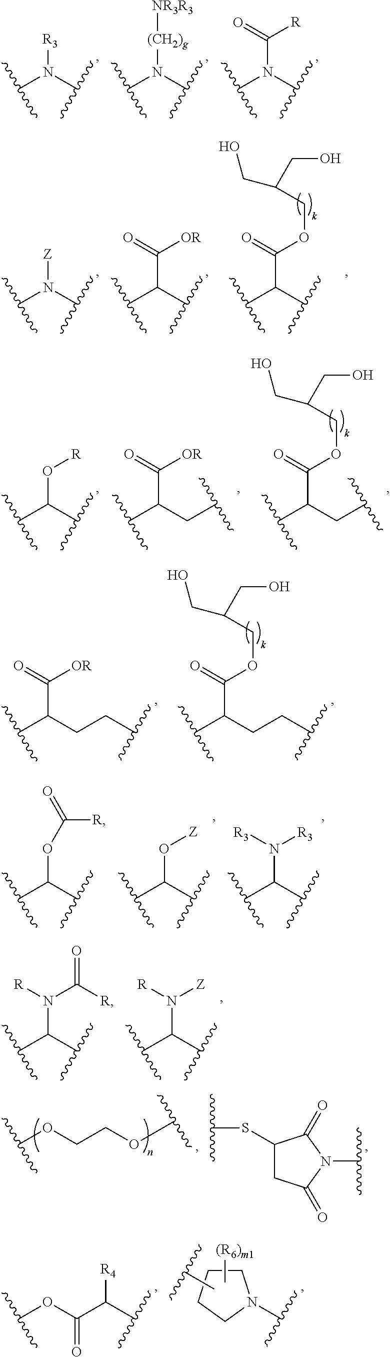 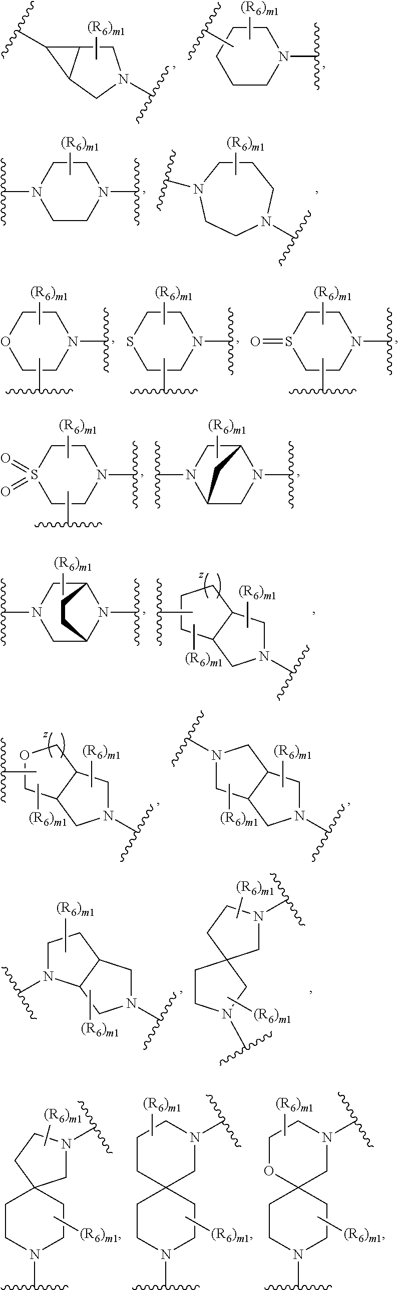 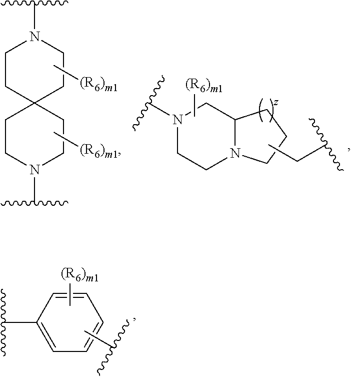
wherein the representation of L is not limited directionally left to right as is depicted, rather either the left side or the right side of L can be bound to the W₁ side of the compound of Formula I;
R₆ is independently —H, —D, —C₁C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁ C₃ alkyl)₂, —SH, —S(C₁-C₃ alkyl), —S(O)C₁-C₃ alkyl;
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl that can be optionally substituted with either O or N and in NR₃R₃ both R₃ when taken together with the nitrogen to which they are attached can form a heterocyclic ring such as a pyrrolidine, piperidine, morpholine, piperazine or pyrrole;
each R₄ is independently e, H or straight or branched C₁-C₁₀ alkyl which can be optionally substituted with OH, NH₂, CO₂R, CONH₂, phenyl, C₆H₄OH, imidazole or amino acids;
each Z is independently —H, or 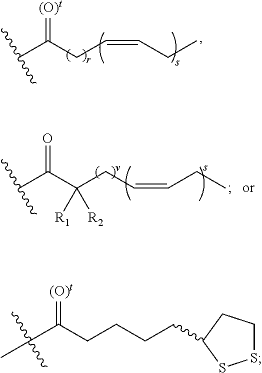
with the proviso that there is at least one 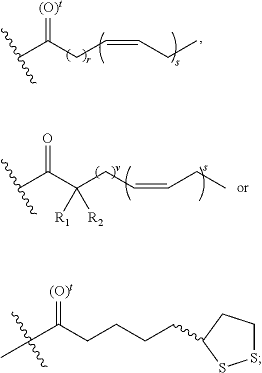
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂,—NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
provided that
when m, n, o, p, and q are each 0, w is 1, W₁ and W₂ are each null, and Z is 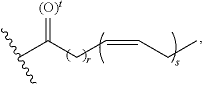
then t must be 0; and
when m, n, o, p, and q are each 0, w is 1, and W₁ and W₂ are each null, then Z must not be

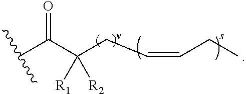

3. A compound of the Formula Ia: 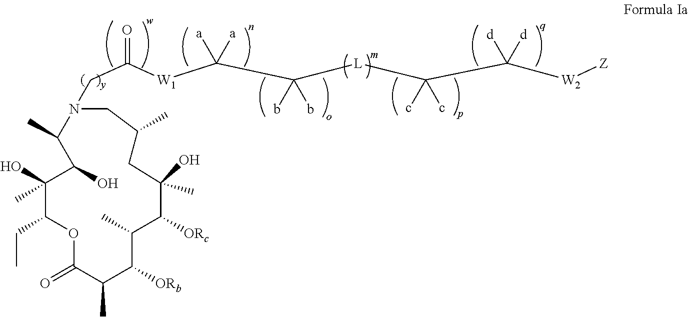
and pharmaceutically acceptable salts, hydrates, solvates, prodrugs, enantiomers, and stereoisomers thereof;
wherein
R_{b} is H, or 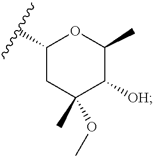 [SMILES: *[C@H]1C[C@@](C)(OC)[C@@H](O)[C@H](C)O1]
R_{c} is H, or 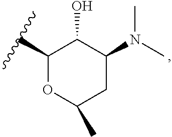 [SMILES: *[C@@H]1O[C@H](C)C[C@H](N(C)C)[C@H]1O]
with the proviso that when R_{c} is H, then R_{b} is H;
W₁ W₂ are each independently null, O, S, NH, NR, or W₁ W₂ can be taken together can form an imidazolidine or piperazine group;
each a, b, c, and d is independently —H, —D, —Ch₃, —OCH₃, —OCH₂CH₃, —C(O)OR, —O-Z, or benzyl, or two of a, b, c, and d can be taken together, along with the single carbon to which they are bound, to form a cycloalkyl or heterocycle;
w is 0 or 1;
y is 0, 1, 2, or 3;
each n, o, p, and q is independently 0, 1 or 2;
L is independently null, —O—, —S—, —S(O)₂-, —S-S—, —(C₁-C₆alkyl)-, —(C₃-C₆cycloalkyl)-, a heterocycle, a heteroaryl, 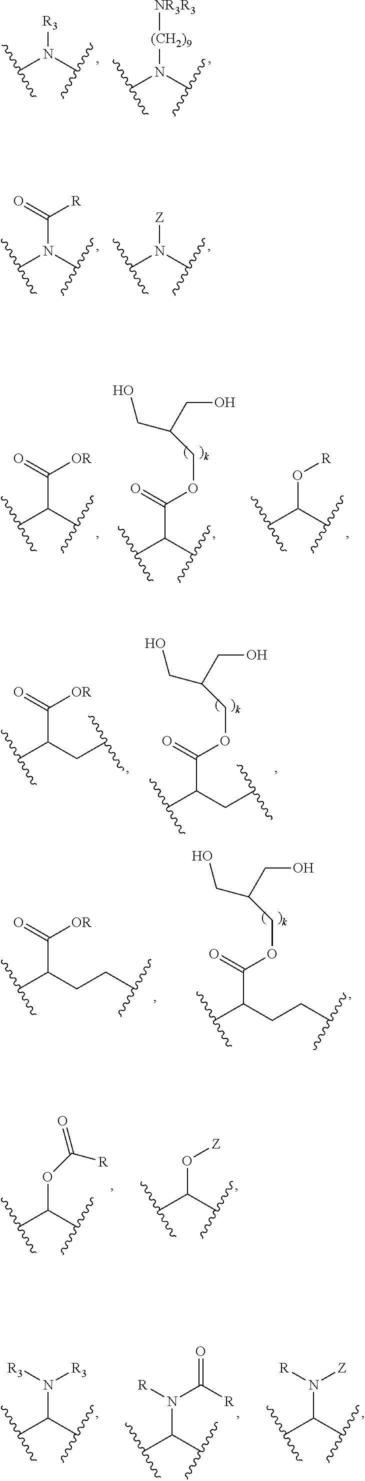 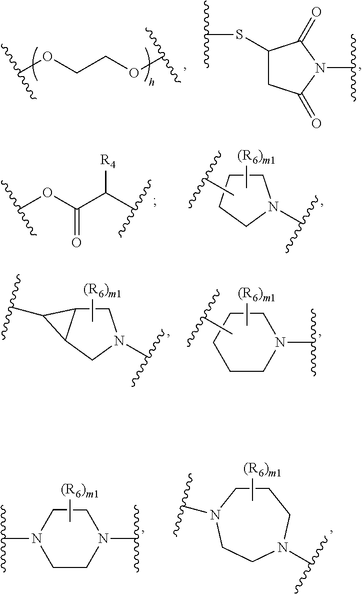 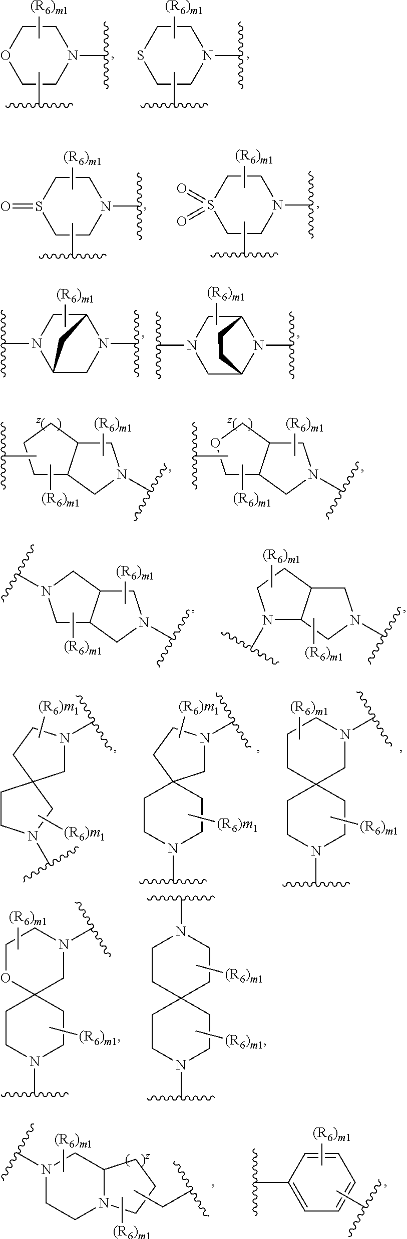
wherein the representation of L is not limited directionally left to right as is depicted, rather either the left side or the right side of L can be bound to the W₁ side of the compound of Formula I;
R₆ is independently —H, —D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2,3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl that can be optionally substituted with either O or N and in NR₃R₃, both or R₃ when taken together with the nitrogen to which they are attached can form a heterocyclic ring such as a pyrrolidine, piperidine, morpholine, piperazine or pyrrole;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, or 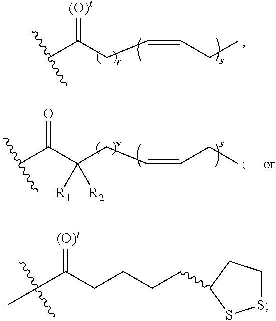
with the proviso that there is at least one 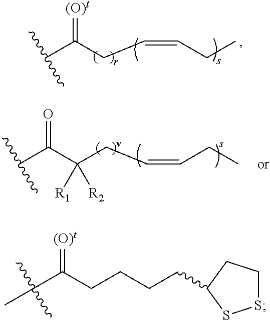
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl), —N(C₁-C₃alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
provided that
when m, n, o, p, and are each 0, w is 1, W₁ and W₂ are each null, and Z is 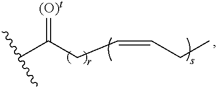
then t must be 0; and
when m, n, o, p, and q are each 0, w is 1, and W₁ and W₂ are each null, then Z must not be

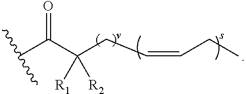

4. The compound of claim 3, wherein Rb and Rc are each H.

5. A compound of the Formula Ib: 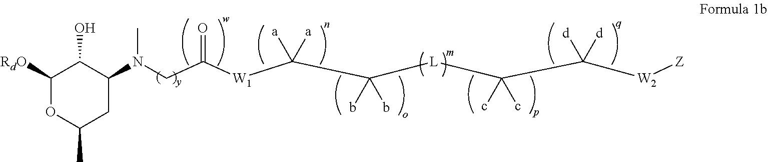
and pharmaceutically acceptable salts, hydrates, solvates, prodrugs, enantiomers, and stereoisomers thereof:
wherein
R_{d} is 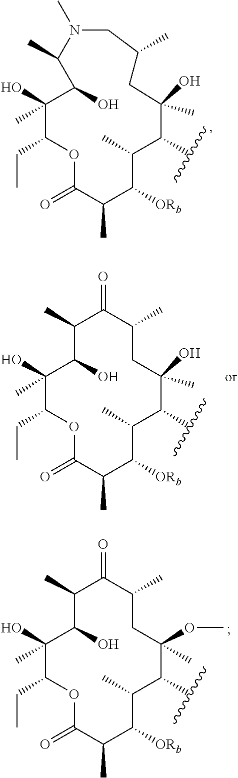
R_{b} is H, or 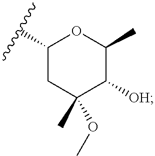 [SMILES: *[C@H]1C[C@@](C)(OC)[C@@H](O)[C@H](C)O1]
W₁ and W₂ are each independently null, O, S, NH, NR, or W₁ and W₂ can be taken together can form an imidazolidine or piperazine group;
each a, b, c, and d is independently —H, —D, —CH₃, —OCH₃, —OCH₂CH₃, —C(O)OR, —O-Z, or benzyl, or two of a, b, c, and d can be taken together, along with the single carbon to which they are bound, to form a cycloalkyl or heterocycle;
w is 0 or 1;
y is 0, 1, 2, or 3;
each n, o, p, and q is independently 0, 1 or 2;
L is independently null, —O—, —S—, —S(O)—, —S(O)₂—, —S-S—,—(C₁-C₆alkyl)-, —(C₃-C₆cycloalkyl)-, a heterocycle, a heteroaryl., 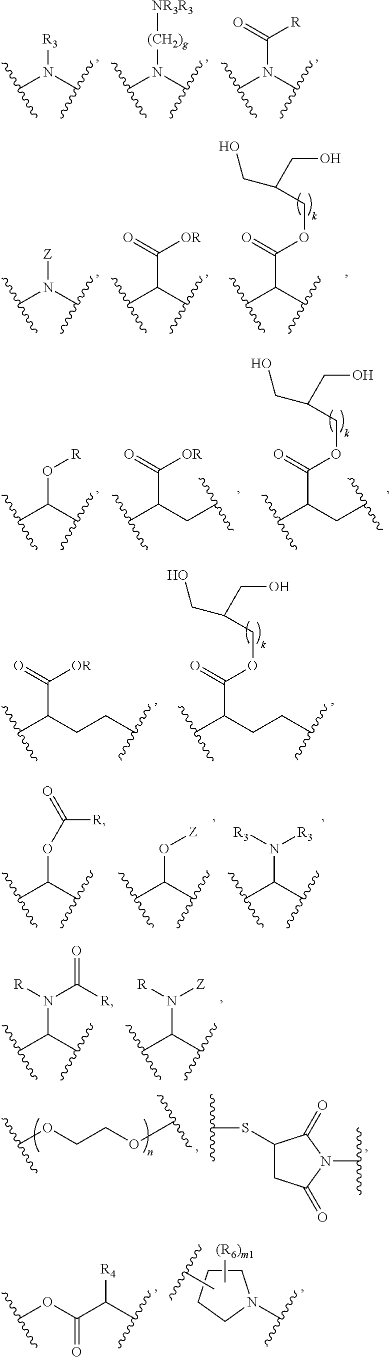 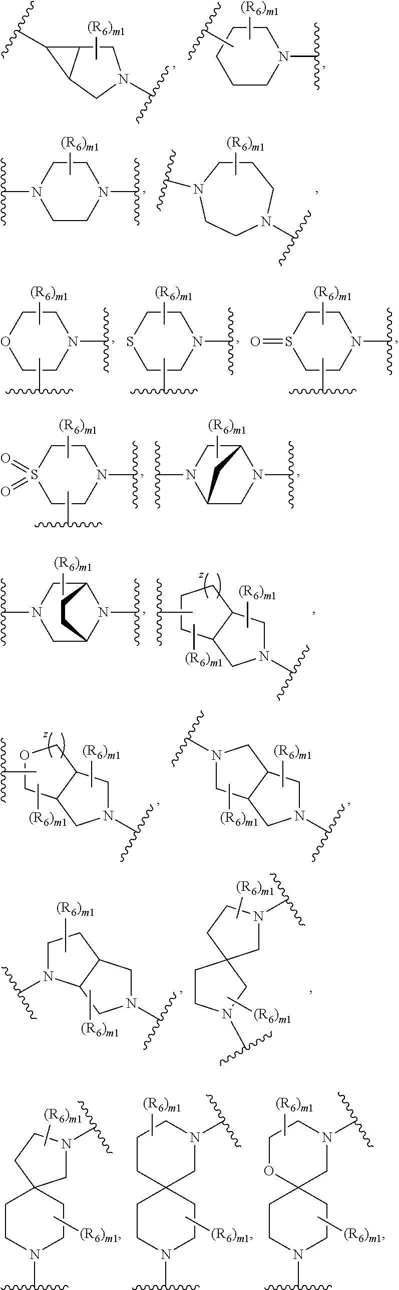 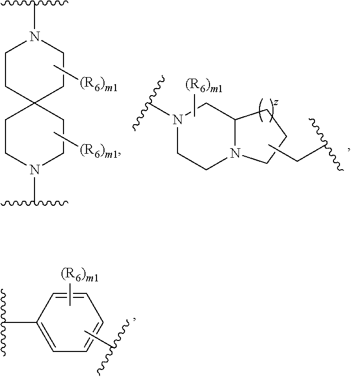
wherein the representation of L is not limited directionally left to right as is depicted, rather either the left side or the right side of L can be bound to the W₁ side of the compound for Formula I;
R₆ is independently —H, —D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m is 0, 1, 2 or 3; is m is more than 1, then L can be the same or different;
m1 is 0, 1, 2 or 3;
k is 0, 1, 2, or 3;
z is 1, 2, or 3;
each R₃ is independently H or C₁-C₆ alkyl that can be optionally substituted with either O or N and NR₃R₃ both R₃ when taken together with the nitrogen to which they are attached can form a heterocyclic ring such as a pyrrolidine, piperidine, morpholine, piperazine or pyrrole;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, or 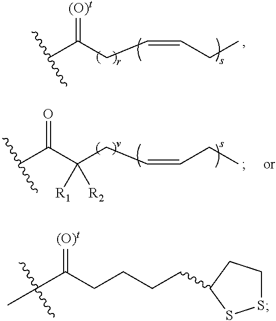
with the proviso that there is at least one 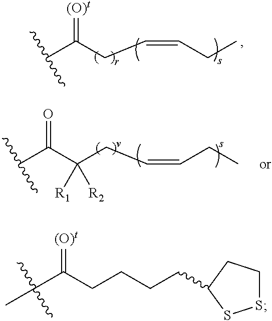
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, -halogen, —Oh, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁C₄ alkyl optionally substituted with OH, or halogen;
provided that
when m, o, p, and q are each 0, w is 1, W₁ and W₂ are each null, and Z is 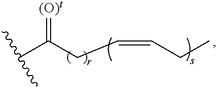
then t must be 0; and
when n, n, o, p, and q are each 0, and W₁ W₂ are each null, then Z must not be

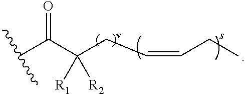

6. A method of treating inflammatory disease, the method comprising administering to a patient in need thereof an effective amount of a molecular conjugate of claim 1.

7. The method of claim 6, wherein the inflammatory disease is selected from rheumatoid arthritis and inflammatory bowel diseases (including colitis and Crohn's disease).

8. The method of claim 6, wherein the inflammatory disease is an inflammatory lung disease.

9. The method of claim 8, wherein the inflammatory lung disease is selected from asthma, adult respiratory distress syndrome, chronic obstructive airway disease, and cystic fibrosis.

10. A method of treating an inflammatory disease, the method comprising administering to a patient in need thereof an effective amount of a compound of claim 2.

11. The method of claim 10, wherein the inflammatory disease is selected from rheumatoid arthritis and inflammatory bowel diseases (including colitis and Crohn's disease).

12. The method of claim 10, wherein the inflammatory disease is an inflammatory lung disease.

13. The method of claim 12, wherein the inflammatory lung disease is selected from asthma, adult respiratory distress syndrome, chronic obstructive airway disease, and cystic fibrosis.

14. A method of treating an inflammatory disease, the method comprising administering to a patient in need thereof an effective amount of a compound of claim 3.

15. The method of claim 14, wherein the inflammatory disease is selected from rheumatoid arthritis and inflammatory bowel diseases (including colitis and Crohn's disease).

16. The method of claim 14, wherein the inflammatory disease is an inflammatory lung disease.

17. The method of claim 16, wherein the inflammatory lung disease is selected from asthma, adult respiratory distress syndrome, chronic obstructive airway disease, and cystic fibrosis.

18. A method of treating an inflammatory disease, the method comprising administering to a patient in need thereof an effective amount of a compound of claim 4.

19. The method of claim 18, wherein the inflammatory disease is selected from rheumatoid arthritis and inflammatory bowel diseases (including colitis and Crohn's disease).

20. The method of claim 18, wherein the inflammatory disease is an inflammatory lung disease.

21. The method of claim 20, wherein the inflammatory lung disease is selected from asthma, adult respiratory distress syndrome, chronic obstructive airway disease, and cystic fibrosis.

22. A method of treating an inflammatory disease, the method comprising administering to a patient in need thereof an effective amount of a compound of claim 5.

23. The method of claim 22, wherein the inflammatory disease is selected from rheumatoid arthritis and inflammatory bowel diseases (including colitis and Crohn's disease).

24. The method of claim 22, wherein the inflammatory disease is an inflammatory lung disease.

25. The method of claim 24, wherein the inflammatory lung disease is selected from asthma, adult respiratory distress syndrome, chronic obstructive airway disease, and cystic fibrosis.

26. A pharmaceutical composition comprising a molecular conjugate of claim 1, and a pharmaceutically acceptable carrier.

27. A pharmaceutical composition comprising a molecular conjugate of claim 2, and a pharmaceutically acceptable carrier.

28. A pharmaceutical composition comprising a molecular conjugate of claim 3, and a pharmaceutically acceptable carrier.

29. A pharmaceutical composition comprising a molecular conjugate of claim 4, and a pharmaceutically acceptable carrier.

30. A pharmaceutical composition comprising a molecular conjugate of claim 5, and a pharmaceutically acceptable carrier.
